(19) United States
(12) Patent Application Publication
(10) Pub. No.: US 2026/0234141 A1
(43) Pub. Date: Aug. 13, 2026

(54) COMPOUNDS AND COMPOSITIONS USEFUL AS DEGRADERS OF MK2 KINASE

(71) Applicant: Celgene Corporation, Princeton, NJ (US)

(72) Inventors: Farid VAN DER MEI, Princeton, NJ (US); Guobin MIAO, Princeton, NJ (US); Lixin QIAO, Andover, MA (US)

(21) Appl. No.: 19/154,292

(22) PCT Filed: Feb. 6, 2024
(86) PCT No.: PCT/US2024/014648

§ 371 Date: Aug. 6, 2025

Related U.S. Application Data
(60) Provisional application No. 63/483,570, filed on Feb. 7, 2023.

(51) Int. Cl. C07D 471/04; A61K 31/496

(52) U.S. Cl. CPC C07D 471/04; A61K 31/496

(57) ABSTRACT

The present disclosure provides compounds and compositions useful for degrading MK2 kinase.

### CROSS-REFERENCE

This application claims the benefit of and priority to U.S. Provisional Patent Application No. 63/483,570 filed on Feb. 7, 2023, the entire contents of which are hereby incorporated by reference herein.

### INCORPORATION BY REFERENCE OF SEQUENCE LISTING

The present application is being filed with a Sequence Listing in electronic format. The Sequence Listing is provided as a file entitled 055920-573001WO_SeqList_ST26.xml, created on Jan. 30, 2024, and is 3 kilobytes in size. The information in electronic format of the Sequence Listing is incorporated herein by reference in its entirety.

### BACKGROUND OF THE INVENTION

The search for new therapeutic agents has been greatly aided in recent years by a better understanding of the structure of enzymes and other biomolecules associated with diseases. One important class of enzymes that has been the subject of extensive study is protein kinases.

Protein kinases constitute a large family of structurally related enzymes that are responsible for the control of a variety of signal transduction processes within the cell. Protein kinases are thought to have evolved from a common ancestral gene due to the conservation of their structure and catalytic function. Almost all kinases contain a similar 250-300 amino acid catalytic domain. The kinases may be categorized into families by the substrates they phosphorylate (e.g., protein-tyrosine, protein-serine/threonine, lipids, etc.).

Protein degradation is a highly regulated and essential process that maintains cellular homeostasis. The selective identification and removal of damaged, misfolded, or excess proteins is achieved via the ubiquitin-proteasome pathway (UPP). The UPP is central to the regulation of almost all cellular processes, including antigen processing, apoptosis, biogenesis of organelles, cell cycling, DNA transcription and repair, differentiation and development, immune response and inflammation, neural and muscular degeneration, morphogenesis of neural networks, modulation of cell surface receptors, ion channels and the secretory pathway, the response to stress and extracellular modulators, ribosome biogenesis and viral infection. Covalent attachment of multiple ubiquitin molecules by an E3 ubiquitin ligase to a terminal lysine residue marks the protein for proteasome degradation, where the protein is digested into small peptides and eventually into its constituent amino acids that serve as building blocks for new proteins. Defective proteasomal degradation has been linked to a variety of disorders including cancer and others.

Cereblon forms part of an E3 ubiquitin ligase complex which interacts with damaged DNA binding protein 1, forming an E3 ubiquitin ligase complex with Cullin 4 and the E2-binding protein ROC1 (known as RBX1) where it functions as a substrate receptor to select proteins for ubiquitination. The binding of lenalidomide to cereblon facilitates subsequent binding of cereblon to Ikaros and Aiolos, leading to their ubiquitination and degradation by the proteasome (see Lu, G. et al. “The myeloma drug lenalidomide promotes the cereblon-dependent destruction of Ikaros proteins” Science, 2014, 343:305-309; Krönke, J. et al. “Lenalidomide causes selective degradation of IKZF1 and IKZF3 in multiple myeloma cells” Science, 2014, 343:301-305).

Mitogen-activated protein kinase-activated protein kinase 2 (MAPKAP K2 or MK2) mediates multiple p38 MAPK-dependent cellular responses. MK2 (SEQ ID NO. 1) is an important intracellular regulator of the production of cytokines, such as tumor necrosis factor alpha (TNF-α), interleukin 6 (IL-6) and interferon gamma (IFNγ), that are involved in many acute and chronic inflammatory diseases, e.g., rheumatoid arthritis and inflammatory bowel disease. MK2 resides in the nucleus of non-stimulated cells and upon stimulation, it translocates to the cytoplasm and phosphorylates and activates tuberin and HSP27. MK2 is also implicated in heart failure, brain ischemic injury, the regulation of stress resistance and the production of TNF-α. (see Deak et al., EMBO. 17:4426-4441 (1998); Shi et al., Biol. Chem. 383:1519-1536 (2002); Staklatvala, Curr. Opin. Pharmacol. 4:372-377 (2004), and Shiroto et al., J. Mol. Cardiol. 38:93-97 (2005)).

SEQ ID NO. 1:
MLSNSQGQSPPVPFPAPAPPPQPPTPALPHPPAQPPPPPPQQFPQFHVKSGLQIKKNAII
DDYKVTSQVLGLGINGKVLQIFNKRTQEKFALKMLQDCPKARREVELHWRASQCPHIV
RIVDVYENLYAGRKCLLIVMECLDGGELFSRIQDRGDQAFTEREASEIMKSIGEAIQYLH
SINIAHRDVKPENLLYTSKRPNAILKLTDFGFAKETTSHNSLTTPCYTPYYVAPEVLGPEK
YDKSCDMWSLGVIMYILLCGYPPFYSNHGLAISPGMKTRIRMGQYEFPNPEWSEVSEEV
KMLIRNLLKTEPTQRMTITEFMNHPWIMQSTKVPQTPLHTSRVLKEDKERWEDVKEEM
TSALATMRVDYEQIKIKKIEDASNPLLLKRRKKARALEAAALAH.

Many diseases are associated with abnormal cellular responses triggered by protein kinase-mediated events as described above. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, and hormone-related diseases. Given the importance of p38α and MK2 in many cellular processes, the activity of both kinases should be controlled. Accordingly, there remains a need to find protein kinase degraders useful as therapeutic agents in the degradation of MK2 and p38α.

### SUMMARY OF THE INVENTION

In certain embodiments, the present disclosure provides a compound of Formula I:

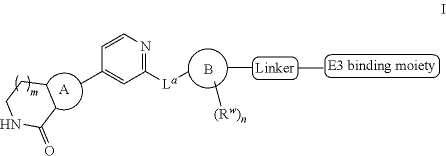

or a pharmaceutically acceptable salt thereof, wherein each of the Linker and E3 binding moiety is as defined infra.

In some embodiments, the present disclosure provides a pharmaceutical composition comprising a compound as described herein, or a pharmaceutically acceptable salt thereof, and a pharmaceutically acceptable carrier, excipient, or vehicle. In some embodiments, a provided pharmaceutical composition is suitable for oral, parenteral, mucosal, transdermal or topical administration.

In some embodiments, the present disclosure provides a method of and degrading MK2 kinase, or a mutant thereof, the method comprising contacting a biological sample with a compound of formula I, or a pharmaceutically acceptable salt thereof.

In some embodiments, the present disclosure provides a method of treating a MK2-mediated disorder, the method comprising administering to a patient in need thereof a compound of formula I, or a pharmaceutically acceptable salt thereof. Such disorders or conditions include, among others, ankylosing spondylitis, rheumatoid arthritis, psoriatic arthritis and psoriasis.

### DETAILED DESCRIPTION OF CERTAIN EMBODIMENTS OF THE INVENTION

### 1. General Description of Compounds of the Invention

In certain embodiments, the present disclosure provides irreversible degraders of MK2. In some embodiments, such compounds include those of the formulae described herein, or a pharmaceutically acceptable salt thereof, wherein each variable is as defined and described herein.

In certain embodiments, the present disclosure provides a compound of Formula I:

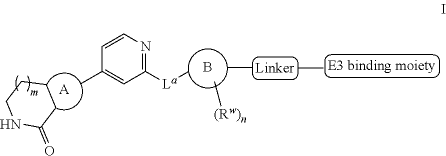

or a pharmaceutically acceptable salt thereof, wherein:
the Linker is a bivalent group;
the E3 binding moiety is a moiety that binds to an E3 ubiquitin ligase protein;
Ring A is

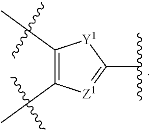

Y¹—N(R)—;
Z¹ is —C(R¹)—
R¹ is selected from hydrogen, halogen, and optionally substituted C₁₋₆ aliphatic;
Ring B is a phenylene;
L^{a} covalent bond;
R^{w} is selected from halogen, —OR, —CN or optionally substituted C₁₋₆ aliphatic;
each R is independently hydrogen or optionally substituted C₁₋₆ aliphatic;
m is 1; and
n is 0, 1, 2, or 3.

### 2. Compounds and Definitions

Compounds of this disclosure include those described generally above, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “carbocyclic”, “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “carbocyclic” (or “cycloaliphatic” or “carbocycle” or “cycloalkyl”) refers to a monocyclic C₃-C₈ hydrocarbon that is completely saturated or that contains one or more units of unsaturation, but which is not aromatic. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups.

Additionally or alternatively, any substitutable nitrogen of a bridged bicyclic group is optionally substituted. Exemplary bridged bicyclics include:

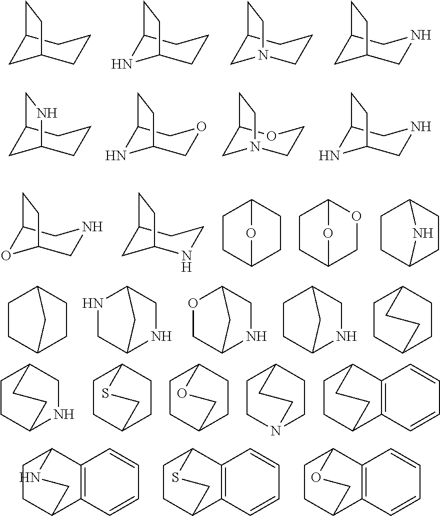

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or, a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

The term “alkylene” refers to a bivalent alkyl group. An “alkylene chain” is a polymethylene group, i.e., —(CH₂)ₙ—, wherein n is a positive integer, and include integers from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present disclosure, “aryl” refers to an aromatic ring system and exemplary groups include phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, including 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Exemplary heteroaryl groups include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Exemplary groups include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, including one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds provided herein may contain “optionally substituted” moieties. In general, the term “substituted,” whether preceded by the term “optionally” or not, means that one or more hydrogens of the designated moiety are replaced with a suitable substituent. “Substituted” applies to one or more hydrogens that are either explicit or implicit from the structure

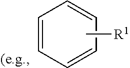

refers to at least

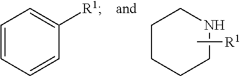

refers to at least

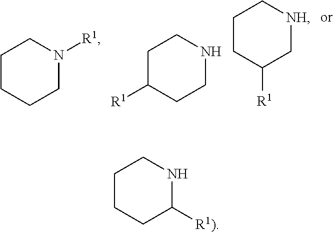

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure include those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}; —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O (“oxo”), ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{†}, -(haloR^{†}), —OH, —OR^{†}, —O(haloR^{†}), —CN, —C(O)OH, —C(O)OR^{†}, —NH₂, —NHR^{†}, —NR^{†}₂, or —NO₂, wherein each R^{†} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of this disclosure. Unless otherwise stated, all tautomeric forms of the compounds provided herein are within the scope of this disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure.

Combinations of substituents and variables envisioned by this disclosure are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

As used herein, the term “inhibitor” is defined as a compound that binds to and/or inhibits the target protein kinase, MK2, with measurable affinity. In certain embodiments, an inhibitor has an IC₅₀ and/or binding constant of less than about 50 μM, less than about 1 μM, less than about 500 nM, less than about 100 nM, or less than about 10 nM.

As used herein the term “degradation”, “degrading”, “MK2 degradation” refers to process by which MK2 proteins are destroyed in a cell in order to maintain protein homeostasis, or an equilibrium of proteins in the human body.

The terms “measurable affinity” and “measurably degrade,” as used herein, means a measurable change in MK2 activity between a sample comprising a compound of the present disclosure, or composition thereof, and MK2, and an equivalent sample comprising MK2, in the absence of said compound, or composition thereof.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof. Inhibition of activity of a protein kinase, for example, MK2 or a mutant thereof, in a biological sample is useful for a variety of purposes that are known to one of skill in the art. Examples of such purposes include, but are not limited to, blood transfusion, organ transplantation, biological specimen storage, and biological assays.

As used herein, a “disease or disorder associated with MK2” or, alternatively, “an MK2-mediated disease or disorder” means any disease or other deleterious condition in which MK2, or a mutant thereof, is known or suspected to play a role.

The term “subject”, as used herein, means a mammal and includes human and animal subjects, such as domestic animals (e.g., horses, dogs, cats, etc.). The terms “subject” and “patient” are used interchangeably. In some embodiments, the “patient” or “subject” means an animal, including a mammal, and a human.

As use herein, the phrase “compound of the disclosure”, “degraders of the disclosure”, “degraders”, refers to those compounds which are disclosed herein, both generically and specifically.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. The amount of compounds of the present disclosure that may be combined with the carrier materials to produce a composition in a single dosage form will vary depending upon the host treated, the particular mode of administration, etc. Provided compositions may further be formulated so that a dosage of between 0.01 to about 100 mg/kg, or about 0.1 mg/kg to about 50 mg/kg, and from about 1 mg/kg to about 25 mg/kg, of subject body weight/day of the degrader can be administered to a patient receiving these compositions to obtain the desired therapeutic effect. The amount of a compound of the present disclosure in the composition will also depend upon the particular compound in the composition.

The expression “unit dosage form” as used herein refers to a physically discrete unit of a provided compound and/or compositions thereof appropriate for the subject to be treated. It will be understood, however, that the total daily usage of the active agent (i.e., compounds and compositions of the present disclosure) will be decided by the attending physician within the scope of sound medical judgment. The specific effective dose level for any particular subject (i.e., patient) or organism will depend upon a variety of factors including the disorder being treated and the severity of the disorder; activity of specific active agent employed; specific composition employed; age, body weight, general health, sex and diet of the subject; time of administration, route of administration, and rate of excretion of the specific active agent employed; duration of the treatment, and like factors well known in the medical arts.

The term “parenteral” as used herein includes subcutaneous, intravenous, intramuscular, intra-articular, intra-synovial, intrasternal, intrathecal, intrahepatic, intralesional and intracranial injection or infusion techniques.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a provided compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a “therapeutically effective amount” is at least a minimal amount of a provided compound, or composition containing a provided compound, which is sufficient for treating one or more symptoms of an MK2-mediated disease or disorder.

### 3. Description of Exemplary Embodiments

In some embodiments, the present disclosure provides a compound of formula I:

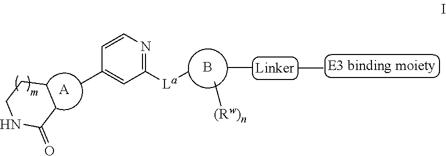

or a pharmaceutically acceptable salt thereof, wherein:
the Linker is a bivalent group;
the E3 binding moiety is a moiety that binds to an E3 ubiquitin ligase protein;
Ring A is selected from

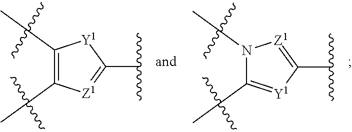

Y¹ is selected from —O—, —S—, and —N(R)—;
each of Y² and Z¹ is selected from —C(R¹)— and —N—;
R¹ is selected from hydrogen, halogen, and optionally substituted C₁₋₆ aliphatic; Ring B is a phenylene, a 5- to 6-membered heteroarylene ring having 1-4 heteroatoms independently selected from nitrogen, oxygen, and sulfur, or a 3- to 7-membered saturated or partially unsaturated heterocyclylene having 1-3 heteroatoms independently selected from nitrogen, oxygen, and sulfur;
L^{a} is selected from a covalent bond, —O—, —S—, —N(R)—, and C₁₋₆ aliphatic;
R^{w} is selected from halogen, —OR, —SR, —CN, —NO₂, —SO₂NR, —SO₂R, —SOR, —C(O)R, —CO₂R, —C(O)N(R)₂, —NRC(O)R, —NRC(O)OR, —NRC(O)N(R)₂, —NRSO₂R, —N(R)₂, or an optionally substituted group selected from the group consisting of C₁₋₆ aliphatic, phenyl, a 3- to 8-membered saturated or partially unsaturated carbocyclic ring, a 4- to 7-membered heterocyclic ring having 1-2 heteroatoms independently selected from nitrogen, oxygen, or sulfur, and a 5- to 6-membered monocyclic heteroaryl ring having 1-4 heteroatoms independently selected from nitrogen, oxygen, or sulfur;
each R is independently hydrogen or optionally substituted C₁₋₆ aliphatic; and each of m and n is 0, 1, 2, or 3.

In some embodiments, Ring A is

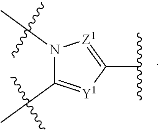

Accordingly, in some embodiments, the present disclosure provides a compound of Formula I-a:

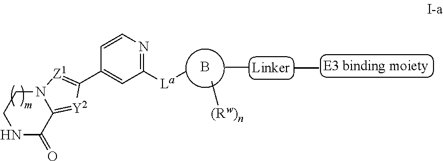

or a pharmaceutically acceptable salt thereof, wherein each of Y², Z¹, L^{a}, Ring B, R^{w}, m, n, Linker, and E3 binding moiety is as defined above and described herein.

In some embodiments, Ring A is

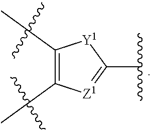

Accordingly, in some embodiments, the present disclosure provides a compound of Formula I-b:

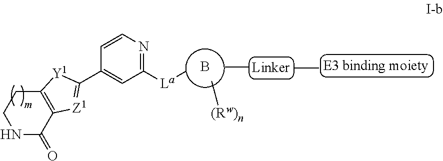

or a pharmaceutically acceptable salt thereof, wherein each of Y¹, Z¹, L^{a}, Ring B, R^{w}, m, n, Linker, and E3 binding moiety is as defined above and described herein.

As defined generally above, Y¹ is selected from —O—, —S—, and —N(R)—.

In some embodiments of Formula I or Formula I-b, Y¹ is —O—. In some embodiments of Formula I or Formula I-b, Y¹ is —S—. In some embodiments of Formula I or Formula I-b, Y¹ is —N(R)—. In some such embodiments, Y¹ is —N(H)—.

As defined generally above, each of Y² and Z¹ is selected from —C(R′)— and —N—. In some embodiments of Formula I or Formula I-a, Y² is —C(R¹)—. In some such embodiments, Y² is —C(H)—. In some embodiments of Formula I or Formula I-a, Y² is —N—.

In some embodiments of any of Formulae I, I-a, and I-b, Z¹ is —C(R¹)—. In some such embodiments, Z¹ is —C(H)—. In some embodiments of any of Formulae I, I-a, and I-b, Z¹ is —N—.

In some embodiments of Formula I or Formula I-a, at least one of Y² and Z¹ is —C(R¹)—.

In some embodiments, the present disclosure provides a compound of Formulae I-a-i, I-a-ii, I-a-Mi, I-a-iv, I-b-i, I-b-ii, I-b-iii, and I-b-iv:

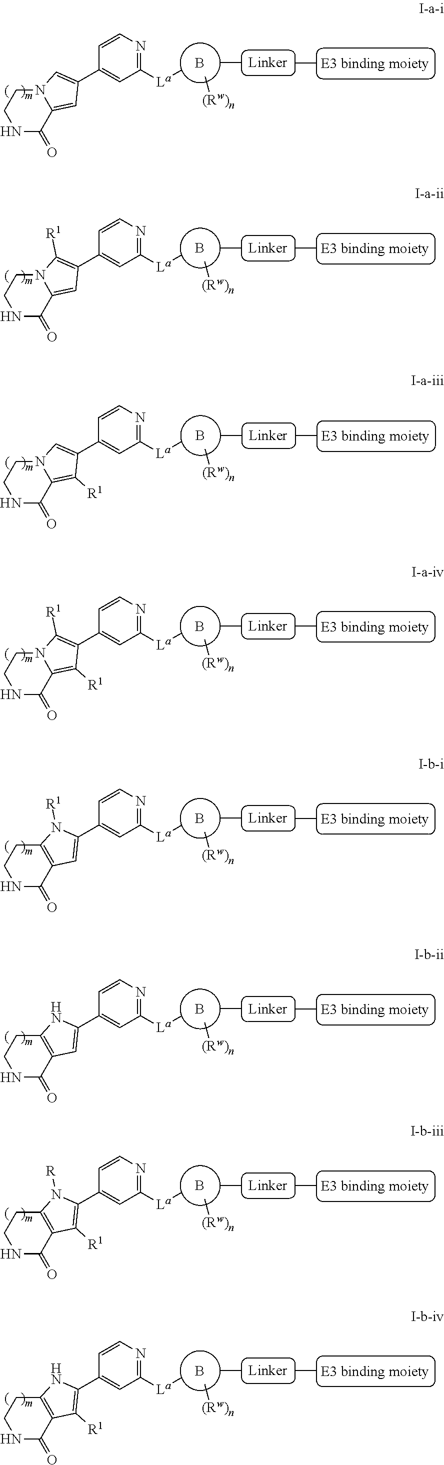

or a pharmaceutically acceptable salt thereof.

As defined generally above, R¹ is selected from hydrogen, halogen, and optionally substituted C₁₋₆ aliphatic. In some embodiments of any Formulae described herein, R¹ is hydrogen. In some embodiments of any Formulae described herein, R¹ is halogen or optionally substituted C₁₋₆ aliphatic. In some embodiments of any Formulae described herein, R¹ is halogen. In some embodiments of any Formulae described herein, R¹ is optionally substituted C₁₋₆ aliphatic. In some such embodiments, R¹ is —CF₃, —CH₂F, —CHF₂, or —CH₂CF₃.

As defined generally above, Ring B is a phenylene, a 5- to 6-membered heteroarylene ring having 1-4 heteroatoms independently selected from nitrogen, oxygen, and sulfur, or a 3- to 7-membered saturated or partially unsaturated heterocyclylene having 1-3 heteroatoms independently selected from nitrogen, oxygen, and sulfur.

In some embodiments of any Formulae described herein, Ring B is phenylene. In some embodiments of any Formulae described herein, Ring B is a 5- to 6-membered heteroarylene ring having 1-4 heteroatoms independently selected from nitrogen, oxygen, and sulfur. In some embodiments of any Formulae described herein, Ring B is a 5-membered heteroarylene ring having 1-4 heteroatoms independently selected from nitrogen, oxygen, and sulfur. In some embodiments of any Formulae described herein, Ring B is a 5-membered heteroarylene ring having 1-3 heteroatoms independently selected from nitrogen, oxygen, and sulfur. In some embodiments of any Formulae described herein, Ring B is a 5-membered heteroarylene ring having 1-2 heteroatoms independently selected from nitrogen, oxygen, and sulfur. In some embodiments of any Formulae described herein, Ring B is a 6-membered heteroarylene ring having 1-2 nitrogen atoms.

In some embodiments of any Formulae described herein, Ring B is a 3- to 7-membered saturated or partially unsaturated heterocyclylene having 1-3 heteroatoms independently selected from nitrogen, oxygen, and sulfur. In some embodiments of any Formulae described herein, Ring B is a 3-membered saturated heterocyclylene having 1 heteroatom selected from nitrogen, oxygen, and sulfur. In some embodiments of any Formulae described herein, Ring B is a 4-membered saturated heterocyclylene having 1 heteroatom selected from nitrogen, oxygen, and sulfur. In some embodiments of any Formulae described herein, Ring B is a 5-membered saturated or partially unsaturated heterocyclylene having 1 heteroatom selected from nitrogen, oxygen, and sulfur. In some embodiments of any Formulae described herein, Ring B is a 6-membered saturated or partially unsaturated heterocyclylene having 1-2 heteroatoms independently selected from nitrogen, oxygen, and sulfur.

As defined generally above, L^{a} is selected from a covalent bond, —O—, —S—, —N(R)—, and C₁₋₆ aliphatic. In some embodiments of any Formulae described herein, L^{a} is a covalent bond. In some embodiments of any Formulae described herein, L^{a} is selected from —O—, —S—, —N(R)—, and C₁₋₆ aliphatic. In some embodiments of any Formulae described herein, L^{a} is —O—. In some embodiments of any Formulae described herein, L^{a} is —S—. In some embodiments of any Formulae described herein, L^{a} is —N(R)—. In some embodiments of any Formulae described herein, L^{a} is C₁₋₆ aliphatic. In some embodiments of any Formulae described herein, L^{a} is C₁₋₄ aliphatic. In some embodiments of any Formulae described herein, L^{a} is C₁₋₂ aliphatic. In some such embodiments, L^{a} is —CH₂—, —CH(CH₃)—, or —CH₂CH₂—.

As defined generally above, R^{w} is selected from halogen, —OR, —SR, —CN, —NO₂, —SO₂NR, —SO₂R, —SOR, —C(O)R, —CO₂R, —C(O)N(R)₂, —NRC(O)R, —NRC(O)OR, —NRC(O)N(R)₂, —NRSO₂R, —N(R)₂, or an optionally substituted group selected from the group consisting of C₁₋₆ aliphatic, phenyl, a 3- to 8-membered saturated or partially unsaturated carbocyclic ring, a 4- to 7-membered heterocyclic ring having 1-2 heteroatoms independently selected from nitrogen, oxygen, or sulfur, and a 5- to 6-membered monocyclic heteroaryl ring having 1-4 heteroatoms independently selected from nitrogen, oxygen, or sulfur. In some embodiments of any Formulae described herein, R^{w} is halogen. In some embodiments of any Formulae described herein, R^{w} is —OR. In some embodiments of any Formulae described herein, R^{w} is —SR. In some embodiments of any Formulae described herein, R^{w} is —CN. In some embodiments of any Formulae described herein, R^{w} is —NO₂. In some embodiments of any Formulae described herein, R^{w} is —SO₂NR. In some embodiments of any Formulae described herein, R^{w} is —SO₂R. In some embodiments of any Formulae described herein, R^{w} is —SOR. In some embodiments of any Formulae described herein, R^{w} is —C(O)R. In some embodiments of any Formulae described herein, R^{w} is —CO₂R. In some embodiments of any Formulae described herein, R^{w} is —C(O)N(R)₂. In some embodiments of any Formulae described herein, R^{w} is —NRC(O)R. In some embodiments of any Formulae described herein, R^{w} is —NRC(O)OR. In some embodiments of any Formulae described herein, R^{w} is —NRC(O)N(R)₂. In some embodiments of any Formulae described herein, R^{w} is —NRSO₂R. In some embodiments of any Formulae described herein, R^{w} is —N(R)₂.

In some embodiments of any Formulae described herein, R^{w} is optionally substituted C₁₋₆ aliphatic. In some embodiments of any Formulae described herein, R^{w} is optionally substituted C₁₋₄ aliphatic. In some embodiments of any Formulae described herein, R^{w} is optionally substituted C₁₋₂ aliphatic. In some embodiments of any Formulae described herein, R^{w} is —CH₃ or CH₂CH₃. In some embodiments of any Formulae described herein, R^{w} is C₁₋₆ aliphatic optionally substituted with halogen. In some such embodiments, R^{w} is —CF₃, —CF₂H, —CFH₂, or —CH₂CF₃.

In some embodiments of any Formulae described herein, R^{w} is optionally substituted phenyl.

In some embodiments of any Formulae described herein, R^{w} is an optionally substituted 3- to 8-membered saturated or partially unsaturated carbocyclic ring. In some embodiments of any Formulae described herein, R^{w} is an optionally substituted 3-membered saturated carbocyclic ring. In some embodiments of any Formulae described herein, R^{w} is an optionally substituted 4-membered saturated carbocyclic ring. In some embodiments of any Formulae described herein, R^{w} is an optionally substituted 5-membered saturated or partially unsaturated carbocyclic ring. In some embodiments of any Formulae described herein, R^{w} is an optionally substituted 6-membered saturated or partially unsaturated carbocyclic ring. In some embodiments of any Formulae described herein, R^{w} is an optionally substituted 7-membered saturated or partially unsaturated carbocyclic ring. In some embodiments of any Formulae described herein, R^{w} is an optionally substituted 8-membered saturated or partially unsaturated carbocyclic ring.

In some embodiments of any Formulae described herein, R^{w} is an optionally substituted 4- to 7-membered heterocyclic ring having 1-2 heteroatoms independently selected from nitrogen, oxygen, or sulfur. In some embodiments of any Formulae described herein, R^{w} is an optionally substituted 4-membered heterocyclic ring having 1 heteroatom selected from nitrogen, oxygen, or sulfur. In some embodiments of any Formulae described herein, R^{w} is an optionally substituted 5-membered heterocyclic ring having 1-2 heteroatoms independently selected from nitrogen, oxygen, or sulfur. In some embodiments of any Formulae described herein, R^{w} is an optionally substituted 6-membered heterocyclic ring having 1-2 heteroatoms independently selected from nitrogen, oxygen, or sulfur. In some embodiments of any Formulae described herein, R^{w} is an optionally substituted 7-membered heterocyclic ring having 1-2 heteroatoms independently selected from nitrogen, oxygen, or sulfur.

In some embodiments of any Formulae described herein, R^{w} is an optionally substituted 5- to 6-membered monocyclic heteroaryl ring having 1-4 heteroatoms independently selected from nitrogen, oxygen, or sulfur. In some embodiments of any Formulae described herein, R^{w} is an optionally substituted 5-membered monocyclic heteroaryl ring having 1-4 heteroatoms independently selected from nitrogen, oxygen, or sulfur. In some embodiments of any Formulae described herein, R^{w} is an optionally substituted 5-membered monocyclic heteroaryl ring having 1-3 heteroatoms independently selected from nitrogen, oxygen, or sulfur. In some embodiments of any Formulae described herein, R^{w} is an optionally substituted 5-membered monocyclic heteroaryl ring having 1-2 heteroatoms independently selected from nitrogen, oxygen, or sulfur. In some embodiments of any Formulae described herein, R^{w} is an optionally substituted 6-membered monocyclic heteroaryl ring having 1-2 nitrogen atoms.

As defined generally above, each R is independently hydrogen or optionally substituted C₁₋₆ aliphatic. In some embodiments of any Formulae described herein, R is hydrogen. In some embodiments of any Formulae described herein, R is optionally substituted C₁₋₆ aliphatic. In some embodiments of any Formulae described herein, R is optionally substituted C₁₋₄ aliphatic. In some embodiments of any Formulae described herein, R is optionally substituted C₁₋₂ aliphatic. In some such embodiments, R is —CH₃, —CH₂CH₃, —CF₃, —CF₂H, —CFH₂, or —CH₂CF₃.

As defined generally above, each of m and n is 0, 1, 2, or 3. In some embodiments of any Formulae described herein, m is 0. In some embodiments of any Formulae described herein, m is 1. In some embodiments of any Formulae described herein, m is 2. In some embodiments of any Formulae described herein, m is 3. In some embodiments of any Formulae described herein, n is 0. In some embodiments of any Formulae described herein, n is 1. In some embodiments of any Formulae described herein, n is 2. In some embodiments of any Formulae described herein, n is 3.

In some embodiments of any Formulae described herein, m is 0 and n is 0. In some embodiments of any Formulae described herein, m is 0 and n is 1. In some embodiments of any Formulae described herein, m is 0 and n is 2. In some embodiments of any Formulae described herein, m is 0 and n is 3. In some embodiments of any Formulae described herein, m is 1 and n is 0. In some embodiments of any Formulae described herein, m is 1 and n is 1. In some embodiments of any Formulae described herein, m is 1 and n is 2. In some embodiments of any Formulae described herein, m is 1 and n is 3.

In some embodiments of any Formulae described herein, m is 2 and n is 0. In some embodiments of any Formulae described herein, m is 2 and n is 1. In some embodiments of any Formulae described herein, m is 2 and n is 2. In some embodiments of any Formulae described herein, m is 2 and n is 3.

In some embodiments of any Formulae described herein, m is 3 and n is 0. In some embodiments of any Formulae described herein, m is 3 and n is 1. In some embodiments of any Formulae described herein, m is 3 and n is 2. In some embodiments of any Formulae described herein, m is 3 and n is 3.

### Linkers

As defined above, the Linker is a bivalent group that links the E3 binding moiety to the rest of the compound. In some embodiments, the Linker is an optionally substituted bivalent C₂₋₂₀ straight or branched aliphatic chain, wherein one, two, three, four, or five methylene units of the aliphatic chain are optionally and independently replaced by a group selected from —N(R)—, —C(O)—, —C(O)N(R)—, —a bivalent 3- to 6-membered monocyclic saturated ring having 0-2 heteroatoms independently selected from nitrogen, a bivalent 6- to 8-membered saturated or partially unsaturated bridged bicyclic, a fused bicyclic or spirofused heterocyclic ring having 1-3 heteroatoms independently selected from nitrogen, each monocyclic ring, bridged bicyclic ring, fused bicyclic ring, spirofused ring, or phenylene is substituted by 0-4 instances of R^{L}, wherein R^{L} is independently selected from halogen, —OR, —SR, —CN, —NO₂, —SO₂NR, —SO₂R, —SOR, —C(O)R, —CO₂R, —C(O)N(R)₂, —NRC(O)R, —NRC(O)OR, —NRC(O)N(R)₂, —NRSO₂R, —N(R)₂, or an optionally substituted group selected from the group consisting of C₁₋₆ aliphatic, phenyl, a 3- to 8-membered saturated or partially unsaturated carbocyclic ring, a 4- to 7-membered heterocyclic ring having 1-2 heteroatoms independently selected from nitrogen, oxygen, or sulfur, and a 5- to 6-membered monocyclic heteroaryl ring having 1-4 heteroatoms independently selected from nitrogen, oxygen, or sulfur. In some embodiments, the Linker is an optionally substituted bivalent C₂₋₂₀ straight or branched aliphatic chain, wherein two, three, or four methylene units of the aliphatic chain are optionally and independently replaced by a group selected from —N(R)—, —O—, —C(O)—, —N(R)C(O)—, —C(O)N(R)—, —OC(O)—, —C(O)O—, and a bivalent 3- to 6-membered monocyclic saturated or partially unsaturated ring having 0-2 heteroatoms independently selected from nitrogen, oxygen, and sulfur, wherein the monocyclic ring is substituted by 0-4 instances of R^{L}. In some embodiments, the Linker is an optionally substituted bivalent C₂₋₂₀ straight or branched aliphatic chain, wherein two, three, four, or five methylene units of the aliphatic chain are optionally and independently replaced by a group selected from —C(O)N(R)—, —N(R)—, —C(O)—,

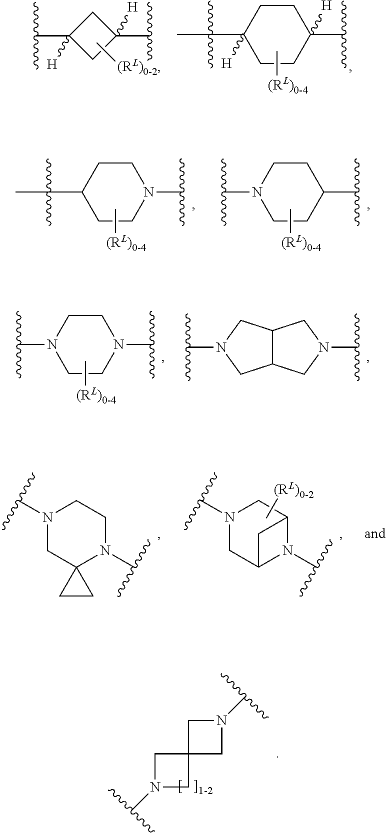

In some embodiments, the Linker is an optionally substituted bivalent C₂₋₂₀ straight or branched aliphatic chain, wherein two, three, four, or five methylene units of the aliphatic chain are optionally and independently replaced by a group selected from —C(O)N(R)—, —N(R)—, —C(O)—,

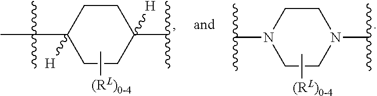

In some embodiments, the Linker is an optionally substituted bivalent C₃₋₁₇ straight or branched aliphatic chain, wherein one, two, three, four, or five methylene units of the aliphatic chain are optionally and independently replaced by a group selected from —N(R)—, —O—, —C(O)—, —N(R)C(O)—, —C(O)N(R)—, —OC(O)—, —C(O)O—, a bivalent 3- to 6-membered monocyclic saturated or partially unsaturated ring having 0-2 heteroatoms independently selected from nitrogen, oxygen, and sulfur, a bivalent 6- to 8-membered saturated or partially unsaturated bridged bicyclic, fused bicyclic or spirofused heterocyclic ring having 1-3 heteroatoms independently selected from nitrogen, oxygen, and sulfur, and phenylene, wherein each monocyclic ring, bridged bicyclic ring, fused bicyclic ring, spirofused ring, or phenylene is substituted by 0-4 instances of R^{L}. In some embodiments, the Linker is an optionally substituted bivalent C₃₋₁₇ straight or branched aliphatic chain, wherein two, three, or four methylene units of the aliphatic chain are optionally and independently replaced by a group selected from —N(R)—, —O—, —C(O)—, —N(R)C(O)—, —C(O)N(R)—, —OC(O)—, —C(O)O—, and a bivalent 3- to 6-membered monocyclic saturated or partially unsaturated ring having 0-2 heteroatoms independently selected from nitrogen, oxygen, and sulfur, wherein the monocyclic ring is substituted by 0-4 instances of R^{L}. In some embodiments, the Linker is an optionally substituted bivalent C₃₋₁₇ straight or branched aliphatic chain, wherein two, three, four, or five methylene units of the aliphatic chain are optionally and independently replaced by a group selected from —C(O)N(R)—, —N(R)—, —C(O)—,

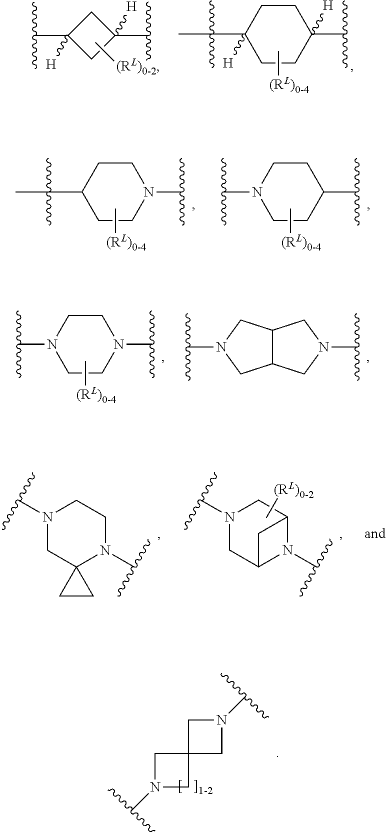

In some embodiments, the Linker is an optionally substituted bivalent C₃₋₁₇ straight or branched aliphatic chain, wherein two, three, four, or five methylene units of the aliphatic chain are optionally and independently replaced by a group selected from —C(O)N(R)—, —N(R)—, —C(O)—,

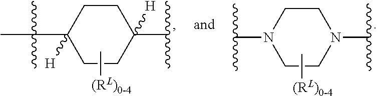

In some embodiments, the Linker is an optionally substituted bivalent C₂₋₁₀ straight or branched aliphatic chain, wherein one, two, three, four, or five methylene units of the aliphatic chain are optionally and independently replaced by a group selected from —N(R)—, —O—, —C(O)—, —N(R)C(O)—, —C(O)N(R)—, —OC(O)—, —C(O)O—, a bivalent 3- to 6-membered monocyclic saturated or partially unsaturated ring having 0-2 heteroatoms independently selected from nitrogen, oxygen, and sulfur, a bivalent 6- to 8-membered saturated or partially unsaturated bridged bicyclic, fused bicyclic or spirofused heterocyclic ring having 1-3 heteroatoms independently selected from nitrogen, oxygen, and sulfur, and phenylene, wherein each monocyclic ring, bridged bicyclic ring, fused bicyclic ring, spirofused ring, or phenylene is substituted by 0-4 instances of R^{L}. In some embodiments, the Linker is an optionally substituted bivalent C₂₋₁₀ straight or branched aliphatic chain, wherein two, three, or four methylene units of the aliphatic chain are optionally and independently replaced by a group selected from —N(R)—, —O—, —C(O)—, —N(R)C(O)—, —C(O)N(R)—, —OC(O)—, —C(O)O—, and a bivalent 3- to 6-membered monocyclic saturated or partially unsaturated ring having 0-2 heteroatoms independently selected from nitrogen, oxygen, and sulfur, wherein the monocyclic ring is substituted by 0-4 instances of R^{L}. In some embodiments, the Linker is an optionally substituted bivalent C₂₋₁₀ straight or branched aliphatic chain, wherein two, three, four, or five methylene units of the aliphatic chain are optionally and independently replaced by a group selected from —C(O)N(R)—, —N(R)—, —C(O)—,

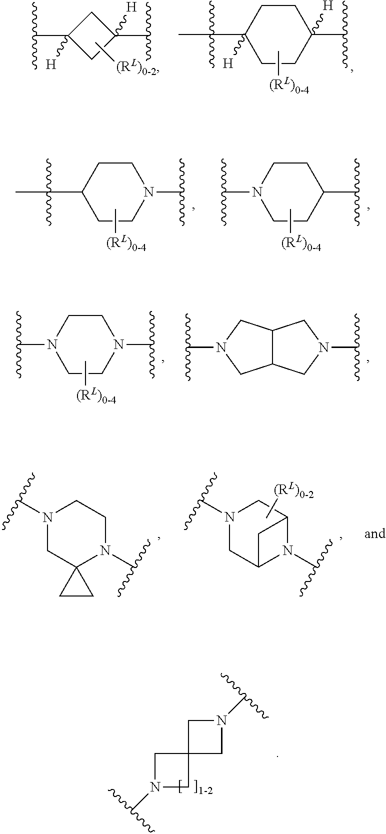

In some embodiments, the Linker is an optionally substituted bivalent C₂₋₁₀ straight or branched aliphatic chain, wherein two, three, four, or five methylene units of the aliphatic chain are optionally and independently replaced by a group selected from —C(O)N(R)—, —N(R)—, —C(O)—,

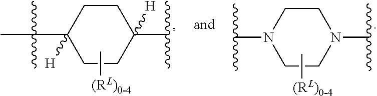

In some embodiments, the Linker is an optionally substituted bivalent C₂₋₆ straight or branched aliphatic chain, wherein one, two, three, four, or five methylene units of the aliphatic chain are optionally and independently replaced by a group selected from —N(R)—, —O—, —C(O)—, —N(R)C(O)—, —C(O)N(R)—, —OC(O)—, —C(O)O—, a bivalent 3- to 6-membered monocyclic saturated or partially unsaturated ring having 0-2 heteroatoms independently selected from nitrogen, oxygen, and sulfur, a bivalent 6- to 8-membered saturated or partially unsaturated bridged bicyclic, fused bicyclic or spirofused heterocyclic ring having 1-3 heteroatoms independently selected from nitrogen, oxygen, and sulfur, and phenylene, wherein each monocyclic ring, bridged bicyclic ring, fused bicyclic ring, spirofused ring, or phenylene is substituted by 0-4 instances of R^{L}. In some embodiments, the Linker is an optionally substituted bivalent C₂₄ straight or branched aliphatic chain, wherein two, three, or four methylene units of the aliphatic chain are optionally and independently replaced by a group selected from —N(R)—, —O—, —C(O)—, —N(R)C(O)—, —C(O)N(R)—, —OC(O)—, —C(O)O—, and a bivalent 3- to 6-membered monocyclic saturated or partially unsaturated ring having 0-2 heteroatoms independently selected from nitrogen, oxygen, and sulfur, wherein the monocyclic ring is substituted by 0-4 instances of R^{L}. In some embodiments, the Linker is an optionally substituted bivalent C₂₋₆ straight or branched aliphatic chain, wherein two, three, four, or five methylene units of the aliphatic chain are optionally and independently replaced by a group selected from —C(O)N(R)—, —N(R)—, —C(O)—,

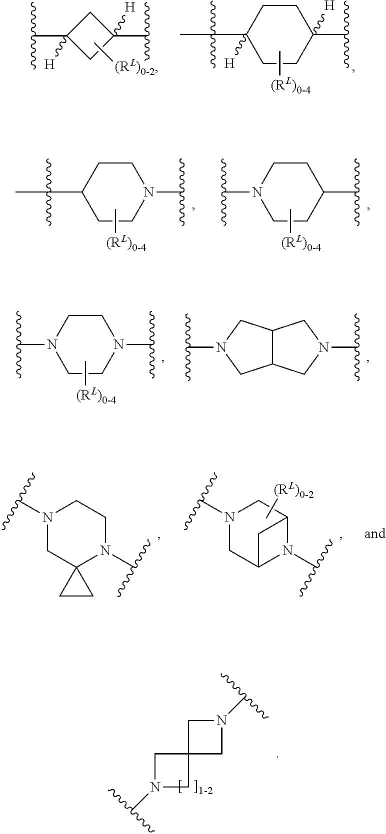

In some embodiments, the Linker is an optionally substituted bivalent C₂₋₆ straight or branched aliphatic chain, wherein two, three, four, or five methylene units of the aliphatic chain are optionally and independently relaced by a group selected from —C(O)N(R)—, —N(R)—, —C(O)—,

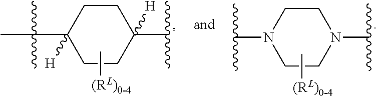

In some embodiments, the Linker is an optionally substituted bivalent C₄ straight or branched aliphatic chain, wherein one, two, three, four, or five methylene units of the aliphatic chain are optionally and independently replaced by a group selected from —N(R)—, —O—, —C(O)—, —N(R)C(O)—, —C(O)N(R)—, —OC(O)—, —C(O)O—, a bivalent 3- to 6-membered monocyclic saturated or partially unsaturated ring having 0-2 heteroatoms independently selected from nitrogen, oxygen, and sulfur, a bivalent 6- to 8-membered saturated or partially unsaturated bridged bicyclic, fused bicyclic or spirofused heterocyclic ring having 1-3 heteroatoms independently selected from nitrogen, oxygen, and sulfur, and phenylene, wherein each monocyclic ring, bridged bicyclic ring, fused bicyclic ring, spirofused ring, or phenylene is substituted by 0-4 instances of R^{L}. In some embodiments, the Linker is an optionally substituted bivalent C₄₋₆ straight or branched aliphatic chain, wherein two, three, or four methylene units of the aliphatic chain are optionally and independently replaced by a group selected from —N(R)—, —O—, —C(O)—, —N(R)C(O)—, —C(O)N(R)—, —OC(O)—, —C(O)O—, and a bivalent 3- to 6-membered monocyclic saturated or partially unsaturated ring having 0-2 heteroatoms independently selected from nitrogen, oxygen, and sulfur, wherein the monocyclic ring is substituted by 0-4 instances of R^{L}. In some embodiments, the Linker is an optionally substituted bivalent C₄ straight or branched aliphatic chain, wherein two, three, four, or five methylene units of the aliphatic chain are optionally and independently replaced by a group selected from —C(O)N(R)—, —N(R)—, —C(O)—,

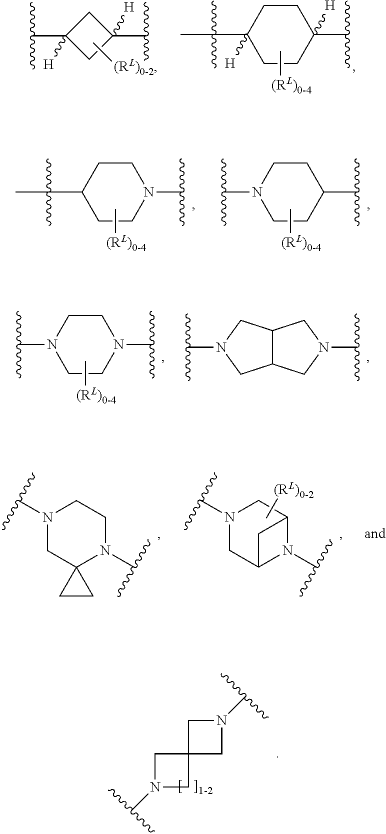

In some embodiments, the Linker is an optionally substituted bivalent C₄₋₆ straight or branched aliphatic chain, wherein two, three, four, or five methylene units of the aliphatic chain are optionally and independently replaced by a group selected from —C(O)N(R)—, —N(R)—, —C(O)—,

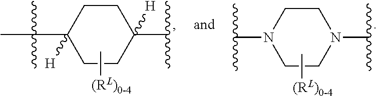

In some embodiments, the Linker is selected from the group consisting of:

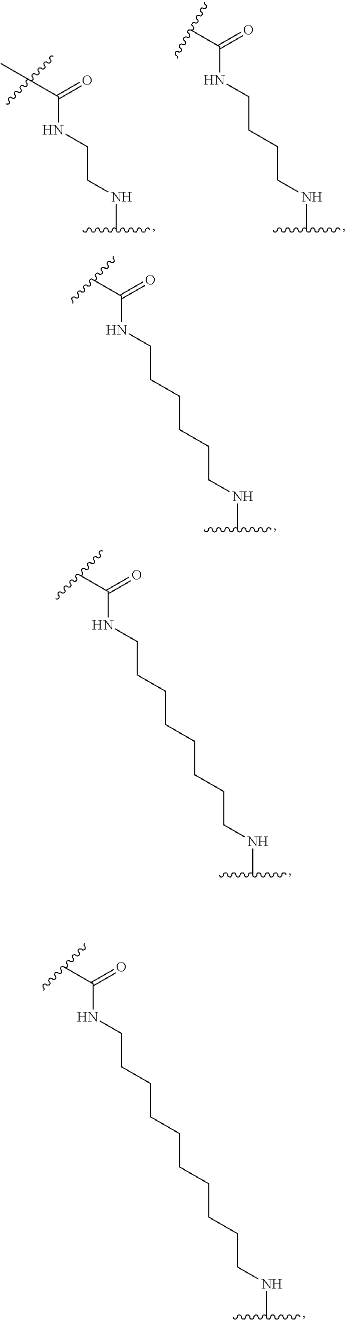 [SMILES: *NCCNC(*)=O ; *NCCCCNC(*)=O ; *NCCCCCCNC(*)=O ; *NCCCCCCCCNC(*)=O ; *NCCCCCCCCCCNC(*)=O]

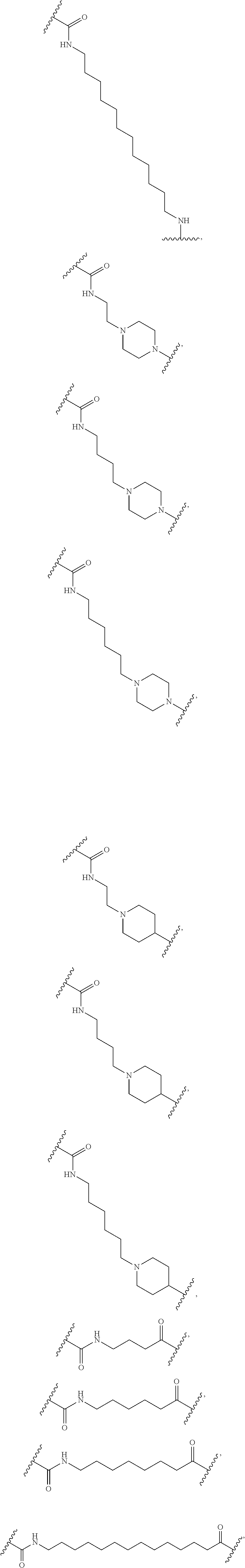

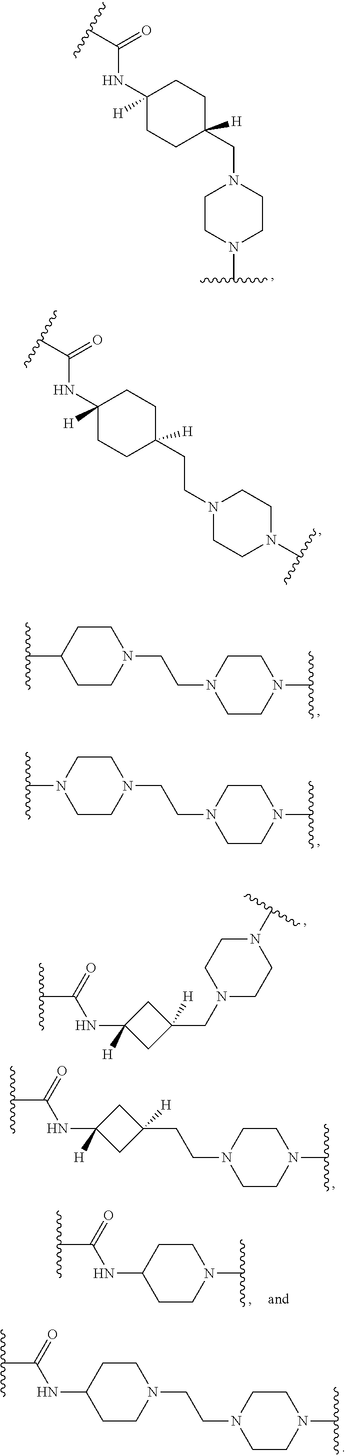

In some embodiments, the Linker is selected from the group consisting of:

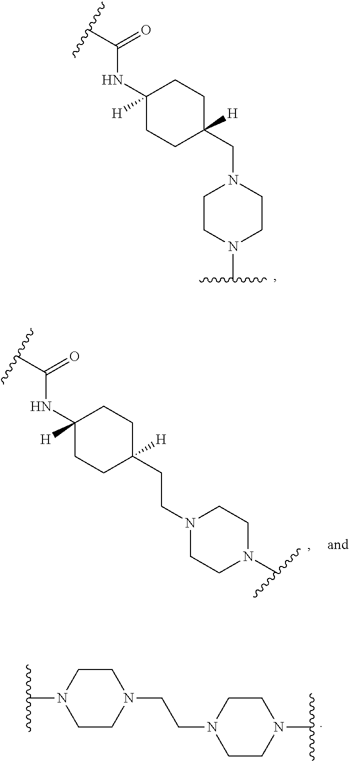 [SMILES: *C(=O)N[C@H]1CC[C@H](CN2CCN(*)CC2)CC1 ; *C(=O)N[C@H]1CC[C@H](CCN2CCN(*)CC2)CC1 ; *N1CCN(CCN2CCN(*)CC2)CC1]

### E3 Binding Moieties

The proteasome is a large protein complex responsible for degradation of intracellular proteins. Polymerization of ubiquitin, a key molecule known to work in concert with the proteasome, serves as a degradation signal for numerous target proteins; the destruction of a protein is initiated by covalent attachment of a chain consisting of several copies of ubiquitin (more than four ubiquitin molecules), through the concerted actions of a network of proteins, including the E1 (ubiquitin-activating), E2 (ubiquitin-conjugating) and E3 (ubiquitin-ligating) enzymes. The polymerized ubiquitin chain acts as a signal that shuttles the target proteins to the proteasome, where the substrate is proteolytically broken down. The set of E3 proteins is highly diverse, because each E3 enzyme selectively recognizes a protein substrate for ubiquitylation. The ubiquitin-proteasome system (UPS) controls almost all basic cellular processes—such as progression through the cell cycle, signal transduction, cell death, immune responses, metabolism, protein quality control and development—by degrading short-lived regulatory or structurally aberrant proteins. Cereblon (CRBN) is a substrate receptor of the CRL4^{CRBN} E3 ubiquitin ligase and induces cell death by targeting key neo-substrates for ubiquitination and subsequent degradation.

In some embodiments, compounds disclosed herein degrade MK2 kinase via the ubiquitin-proteasome system.

As defined generally above, the E3 binding moiety is a moiety that binds to an E3 ubiquitin ligase protein. In some embodiments, the E3 binding moiety is a cereblon protein binding moiety.

In some embodiments, the cereblon protein binding moiety is selected from:

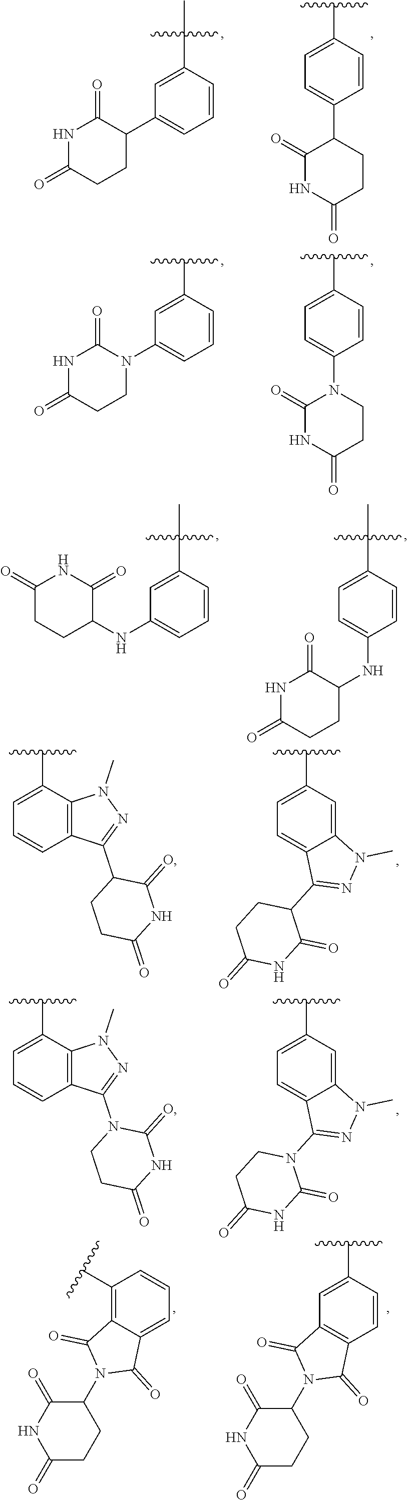

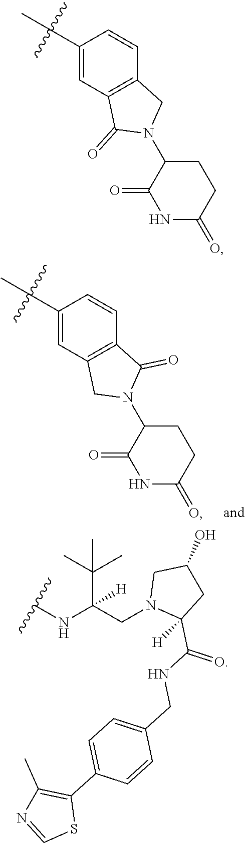

In some embodiments, the cereblon protein binding moiety is selected from:

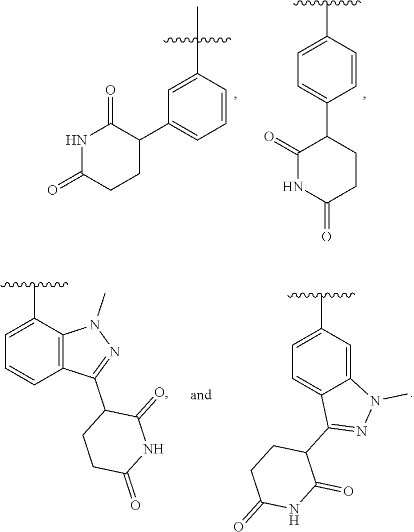

In some embodiments, a compound of Formula I or a pharmaceutically acceptable salt thereof is selected from Table 1.

TABLE 1
Exemplary compounds of Formula I

| | |
|---|---|
| 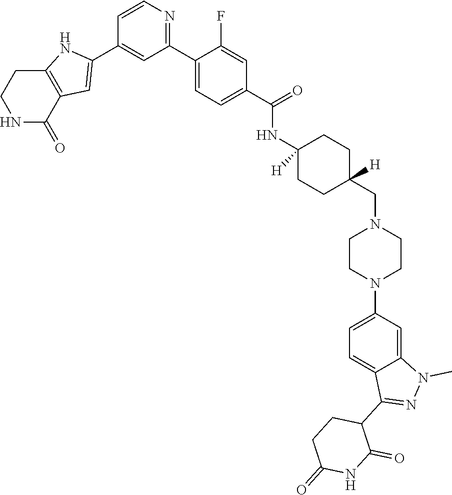 [SMILES: Cn1nc(C2CCC(=O)NC2=O)c2ccc(N3CCN(C[C@H]4CC[C@H](NC(=O)c5ccc(-c6cc(-c7cc8c([nH]7)CCNC8=O)ccn6)c(F)c5)CC4)CC3)cc21] | Ex. 1.5 |
| 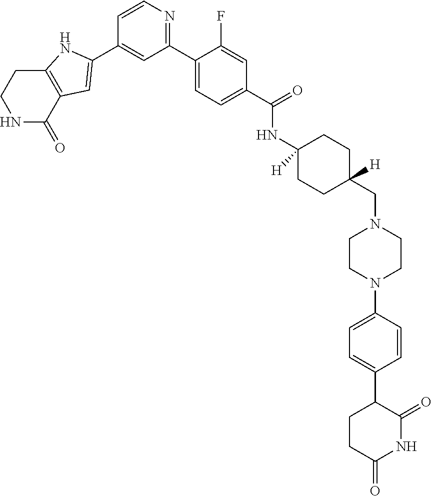 [SMILES: O=C1CCC(c2ccc(N3CCN(C[C@H]4CC[C@H](NC(=O)c5ccc(-c6cc(-c7cc8c([nH]7)CCNC8=O)ccn6)c(F)c5)CC4)CC3)cc2)C(=O)N1] | Ex 1.6 |
| 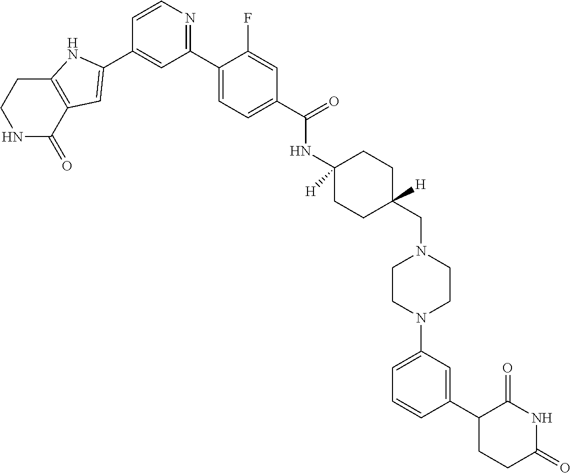 [SMILES: O=C1CCC(c2cccc(N3CCN(C[C@H]4CC[C@H](NC(=O)c5ccc(-c6cc(-c7cc8c([nH]7)CCNC8=O)ccn6)c(F)c5)CC4)CC3)c2)C(=O)N1] | Ex 1.7 |
| 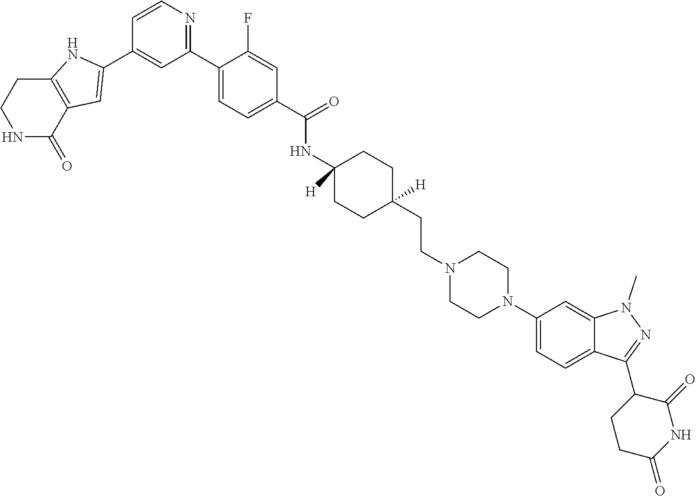 [SMILES: Cn1nc(C2CCC(=O)NC2=O)c2ccc(N3CCN(CC[C@H]4CC[C@H](NC(=O)c5ccc(-c6cc(-c7cc8c([nH]7)CCNC8=O)ccn6)c(F)c5)CC4)CC3)cc21] | Ex 1.8 |
| 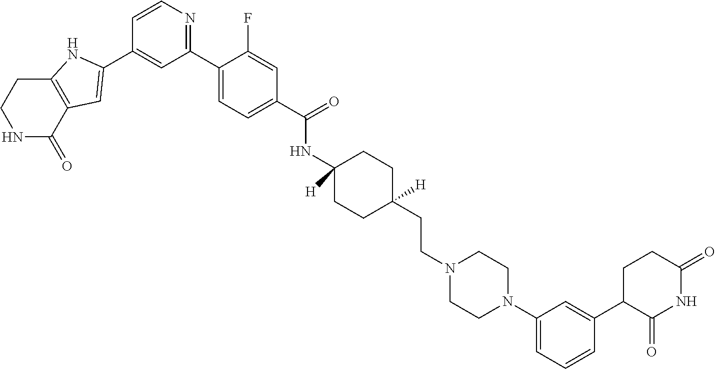 [SMILES: O=C1CCC(c2cccc(N3CCN(CC[C@H]4CC[C@H](NC(=O)c5ccc(-c6cc(-c7cc8c([nH]7)CCNC8=O)ccn6)c(F)c5)CC4)CC3)c2)C(=O)N1] | Ex 1.9 |
| 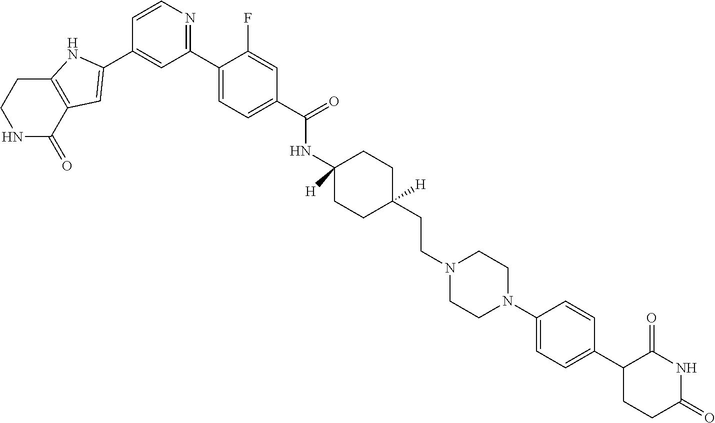 [SMILES: O=C1CCC(c2ccc(N3CCN(CC[C@H]4CC[C@H](NC(=O)c5ccc(-c6cc(-c7cc8c([nH]7)CCNC8=O)ccn6)c(F)c5)CC4)CC3)cc2)C(=O)N1] | Ex 1.10 |
| 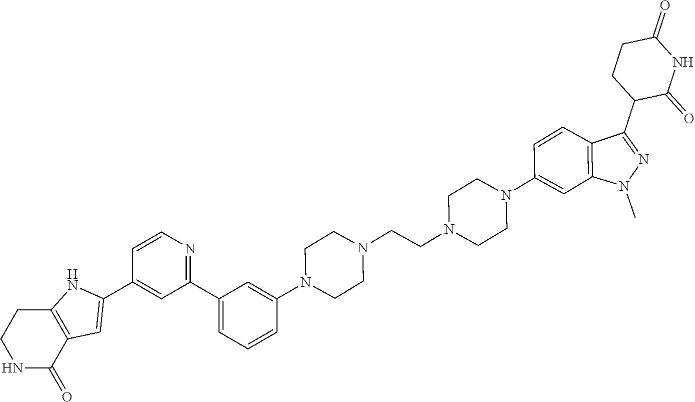 [SMILES: Cn1nc(C2CCC(=O)NC2=O)c2ccc(N3CCN(CCN4CCN(c5cccc(-c6cc(-c7cc8c([nH]7)CCNC8=O)ccn6)c5)CC4)CC3)cc21] | Ex 1.11 |
| 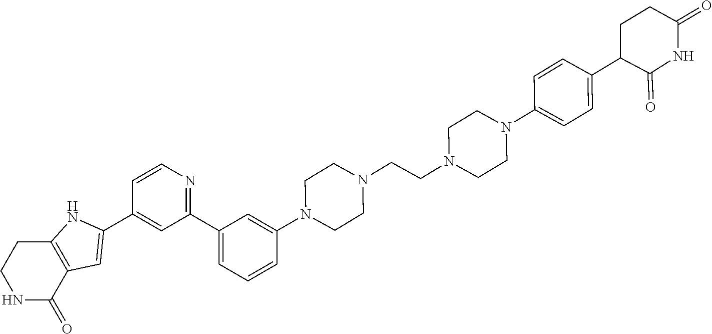 [SMILES: O=C1CCC(c2ccc(N3CCN(CCN4CCN(c5cccc(-c6cc(-c7cc8c([nH]7)CCNC8=O)ccn6)c5)CC4)CC3)cc2)C(=O)N1] | Ex 1.12 |
| 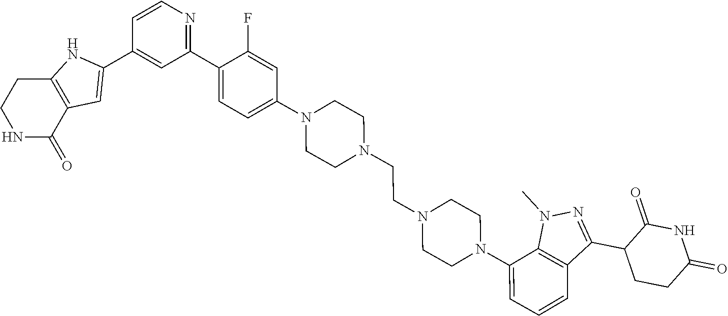 [SMILES: Cn1nc(C2CCC(=O)NC2=O)c2cccc(N3CCN(CCN4CCN(c5ccc(-c6cc(-c7cc8c([nH]7)CCNC8=O)ccn6)c(F)c5)CC4)CC3)c21] | Ex 1.13 |

In some embodiments, the compounds or pharmaceutically acceptable salts thereof of the present disclosure have the Formula:

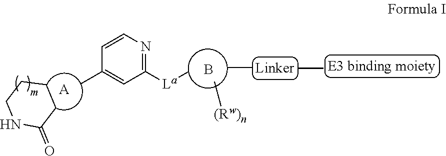

wherein:
the Linker is a bivalent group;
the E3 binding moiety is a moiety that binds to an E3 ubiquitin ligase protein;
Ring A is selected from

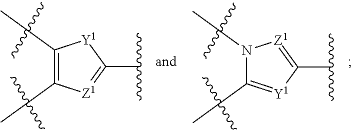

Y¹ is selected from —O—, —S—, and —N(R)—;
each of Y² and Z¹ is selected from —C(R¹)— and —N—;
R¹ is selected from hydrogen, halogen, and optionally substituted C₁₋₆ aliphatic;
Ring B is a phenylene, a 5- to 6-membered heteroarylene ring having 1-4 heteroatoms independently selected from nitrogen, oxygen, and sulfur, or a 3- to 7-membered saturated or partially unsaturated heterocyclylene having 1-3 heteroatoms independently selected from nitrogen, oxygen, and sulfur;
L^{a} is selected from a covalent bond, —O—, —S—, —N(R)—, and C₁₋₆ aliphatic;
R^{w} is selected from halogen, —OR, —SR, —CN, —NO₂, —SO₂NR, —SO₂R, —SOR, —C(O)R, —CO₂R, —C(O)N(R)₂, —NRC(O)R, —NRC(O)OR, —NRC(O)N(R)₂, —NRSO₂R, —N(R)₂, or an optionally substituted group selected from the group consisting of C₁₋₆ aliphatic, phenyl, a 3- to 8-membered saturated or partially unsaturated carbocyclic ring, a 4- to 7-membered heterocyclic ring having 1-2 heteroatoms independently selected from nitrogen, oxygen, or sulfur, and a 5- to 6-membered monocyclic heteroaryl ring having 1-4 heteroatoms independently selected from nitrogen, oxygen, or sulfur;
each R is independently hydrogen or optionally substituted C₁₋₆ aliphatic; and
each of m and n is 0, 1, 2, or 3.

In some embodiments of the compounds of the present disclosure, the Ring A is

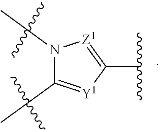

In some embodiments of the disclosure, the compound is a compound of Formula I-a:

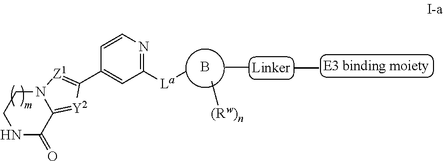

or a pharmaceutically acceptable salt thereof.

In some embodiments of the compounds of the present disclosure Y² is —C(R¹)—.

In some embodiments of the compounds of the present disclosure Z¹ is —C(R¹)—.

In some embodiments of the compounds of the present disclosure, R¹ is hydrogen.

In some embodiments of the compounds of the present disclosure, Y² is —N—.

In some embodiments of the compounds of the present disclosure, Z¹ is —N—.

In some embodiments of the compounds of the present disclosure, the Ring A is

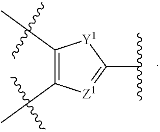

In some embodiments of the disclosure, the compound is a compound of Formula I-b:

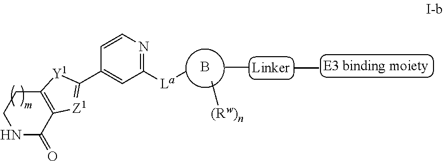

or a pharmaceutically acceptable salt thereof.

In some embodiments of the compounds of the present disclosure, Y¹ is —N(R)—.

In some embodiments of the compounds of the present disclosure, R is hydrogen.

In some embodiments of the compounds of the present disclosure, Y¹ is —S—.

In some embodiments of the compounds of the present disclosure, Y¹ is —O—.

In some embodiments of the compounds of the present disclosure, at least one R¹ is hydrogen.

In some embodiments of the compounds of the present disclosure, at least one R¹ is selected from halogen or optionally substituted C₁₋₆ aliphatic.

In some embodiments of the compounds of the present disclosure, at least one R¹ is optionally substituted C₁₋₆ aliphatic.

In some embodiments of the compounds of the present disclosure, at least one R¹ is selected from —CF₃, —CH₂F, —CHF₂, or —CH₂CF₃.

In some embodiments of the compounds of the present disclosure, at least one R¹ is halogen.

In some embodiments of the compounds of the present disclosure, wherein Ring B is phenylene.

In some embodiments of the compounds of the present disclosure, the Ring B is a 5- to 6-membered heteroarylene ring having 1-4 heteroatoms independently selected from nitrogen, oxygen, and sulfur.

In some embodiments of the compounds of the present disclosure, the Ring B is a 5-membered heteroarylene ring having 1-4 heteroatoms independently selected from nitrogen, oxygen, and sulfur.

In some embodiments of the compounds of the present disclosure, the Ring B is a 5-membered heteroarylene ring having 1-3 heteroatoms independently selected from nitrogen, oxygen, and sulfur.

In some embodiments of the compounds of the present disclosure, the Ring B is a 5-membered heteroarylene ring having 1-2 heteroatoms independently selected from nitrogen, oxygen, and sulfur.

In some embodiments of the compounds of the present disclosure, the Ring B is a 6-membered heteroarylene ring having 1-2 nitrogen atoms.

In some embodiments of the compounds of the present disclosure, the Ring B is a 3- to 7-membered saturated or partially unsaturated heterocyclylene having 1-3 heteroatoms independently selected from nitrogen, oxygen, and sulfur.

In some embodiments of the compounds of the present disclosure, the Ring B is a 3-membered saturated heterocyclylene having 1 heteroatom selected from nitrogen, oxygen, and sulfur.

In some embodiments of the compounds of the present disclosure, the Ring B is a 4-membered saturated heterocyclylene having 1 heteroatom selected from nitrogen, oxygen, and sulfur.

In some embodiments of the compounds of the present disclosure, the Ring B is a 5-membered saturated heterocyclylene having 1 heteroatom selected from nitrogen, oxygen, and sulfur.

In some embodiments of the compounds of the present disclosure, the Ring B is a 6-membered saturated or partially unsaturated heterocyclylene having 1-2 heteroatoms independently selected from nitrogen, oxygen, and sulfur.

In some embodiments of the compounds of the present disclosure, L^{a} is a covalent bond.

In some embodiments of the compounds of the present disclosure, L^{a} is —O—.

In some embodiments of the compounds of the present disclosure, L^{a} is —S—.

In some embodiments of the compounds of the present disclosure, L^{a} is —N(R)—.

In some embodiments of the compounds of the present disclosure, L^{a} is C₁₋₆ aliphatic.

In some embodiments of the compounds of the present disclosure, L^{a} is C₁₋₄ aliphatic.

In some embodiments of the compounds of the present disclosure, L^{a} is C₁₋₂ aliphatic.

In some embodiments of the compounds of the present disclosure, L^{a} is —CH₂—, —CH(CH₃)—, or —CH₂CH₂—.

In some embodiments of the compounds of the present disclosure, R^{w} is halogen.

In some embodiments of the compounds of the present disclosure, R^{w} is —CN.

In some embodiments of the compounds of the present disclosure, R^{w} is —NO₂.

In some embodiments of the compounds of the present disclosure, R^{w} is —OR.

In some embodiments of the compounds of the present disclosure, R^{w} is —SR.

In some embodiments of the compounds of the present disclosure, R^{w} is —SO₂NR.

In some embodiments of the compounds of the present disclosure, R^{w} is —SO₂R.

In some embodiments of the compounds of the present disclosure, R^{w} is —SOR.

In some embodiments of the compounds of the present disclosure, R^{w} is —C(O)R.

In some embodiments of the compounds of the present disclosure, R^{w} is —CO₂R.

In some embodiments of the compounds of the present disclosure, R^{w} is —C(O)N(R)₂.

In some embodiments of the compounds of the present disclosure, R^{w} is —NRC(O)R.

In some embodiments of the compounds of the present disclosure, R^{w} is —NRC(O)OR.

In some embodiments of the compounds of the present disclosure, R^{w} is —NRC(O)N(R)₂.

In some embodiments of the compounds of the present disclosure, R^{w} is —NRSO₂R.

In some embodiments of the compounds of the present disclosure, R^{w} is —N(R)₂.

In some embodiments of the compounds of the present disclosure, R is hydrogen.

In some embodiments of the compounds of the present disclosure, R is optionally substituted C₁₋₆ aliphatic.

In some embodiments of the compounds of the present disclosure, R is optionally substituted C₁₋₄ aliphatic.

In some embodiments of the compounds of the present disclosure, R is optionally substituted C₁₋₂ aliphatic.

In some embodiments of the compounds of the present disclosure, R is selected from —CH₃, —CH₂CH₃, —CF₃, —CF₂H, —CFH₂, or —CH₂CF₃.

In some embodiments of the compounds of the present disclosure, R^{w} is an optionally substituted group selected from the group consisting of C₁₋₆ aliphatic, phenyl, a 3- to 8-membered saturated or partially unsaturated carbocyclic ring, a 4- to 7-membered heterocyclic ring having 1-2 heteroatoms independently selected from nitrogen, oxygen, or sulfur, and a 5- to 6-membered monocyclic heteroaryl ring having 1-4 heteroatoms independently selected from nitrogen, oxygen, or sulfur.

In some embodiments of the compounds of the present disclosure, R^{w} is optionally substituted C₁₋₆ aliphatic.

In some embodiments of the compounds of the present disclosure, R^{w} is optionally substituted C₁₋₄ aliphatic.

In some embodiments of the compounds of the present disclosure, R^{w} is optionally substituted C₁₋₂ aliphatic.

In some embodiments of the compounds of the present disclosure, R^{w} is —CH₃ or CH₂CH₃.

In some embodiments of the compounds of the present disclosure, R^{w} is C₁₋₆ aliphatic optionally substituted with halogen.

In some embodiments of the compounds of the present disclosure, R^{w} is —CF₃, —CF₂H, —CFH₂, or —CH₂CF₃.

In some embodiments of the compounds of the present disclosure, R^{w} is optionally substituted phenyl.

In some embodiments of the compounds of the present disclosure, R^{w} is an optionally substituted 3- to 8-membered saturated or partially unsaturated carbocyclic ring.

In some embodiments of the compounds of the present disclosure, R^{w} is an optionally substituted 3-membered saturated carbocyclic ring.

In some embodiments of the compounds of the present disclosure, R^{w} is an optionally substituted 4-membered saturated carbocyclic ring.

In some embodiments of the compounds of the present disclosure, R^{w} is an optionally substituted 5-membered saturated or partially unsaturated carbocyclic ring.

In some embodiments of the compounds of the present disclosure, R^{w} is an optionally substituted 6-membered saturated or partially unsaturated carbocyclic ring.

In some embodiments of the compounds of the present disclosure, R^{w} is an optionally substituted 7-membered saturated or partially unsaturated carbocyclic ring.

In some embodiments of the compounds of the present disclosure, R^{w} is an optionally substituted 8-membered saturated or partially unsaturated carbocyclic ring.

In some embodiments of the compounds of the present disclosure, R^{w} is an optionally substituted 4- to 7-membered heterocyclic ring having 1-2 heteroatoms independently selected from nitrogen, oxygen, or sulfur.

In some embodiments of the compounds of the present disclosure, R^{w} is an optionally substituted 4-membered heterocyclic ring having 1 heteroatom selected from nitrogen, oxygen, or sulfur.

In some embodiments of the compounds of the present disclosure, R^{w} is an optionally substituted 5-membered heterocyclic ring having 1-2 heteroatoms independently selected from nitrogen, oxygen, or sulfur.

In some embodiments of the compounds of the present disclosure, R^{w} is an optionally substituted 6-membered heterocyclic ring having 1-2 heteroatoms independently selected from nitrogen, oxygen, or sulfur.

In some embodiments of the compounds of the present disclosure, R^{w} is an optionally substituted 7-membered heterocyclic ring having 1-2 heteroatoms independently selected from nitrogen, oxygen, or sulfur.

In some embodiments of the compounds of the present disclosure, R^{w} is an optionally substituted 5- to 6-membered monocyclic heteroaryl ring having 1-4 heteroatoms independently selected from nitrogen, oxygen, or sulfur.

In some embodiments of the compounds of the present disclosure, R^{w} is an optionally substituted 5-membered monocyclic heteroaryl ring having 1-4 heteroatoms independently selected from nitrogen, oxygen, or sulfur.

In some embodiments of the compounds of the present disclosure, R^{w} is an optionally substituted 5-membered monocyclic heteroaryl ring having 1-3 heteroatoms independently selected from nitrogen, oxygen, or sulfur.

In some embodiments of the compounds of the present disclosure, R^{w} is an optionally substituted 5-membered monocyclic heteroaryl ring having 1-2 heteroatoms independently selected from nitrogen, oxygen, or sulfur.

In some embodiments of the compounds of the present disclosure, R^{w} is an optionally substituted 6-membered monocyclic heteroaryl ring having 1-2 nitrogen atoms.

In some embodiments of the compounds of the present disclosure, m is 1.

In some embodiments of the compounds of the present disclosure, wherein n is 0.

In some embodiments of the compounds of the present disclosure, n is 1.

In some embodiments, suitable compounds or pharmaceutically acceptable salt of the disclosure have the Formulae I-a-i, I-a-ii, I-a-iii, I-a-iv, I-b-i, I-b-ii, I-b-ii, and I-b-iv:

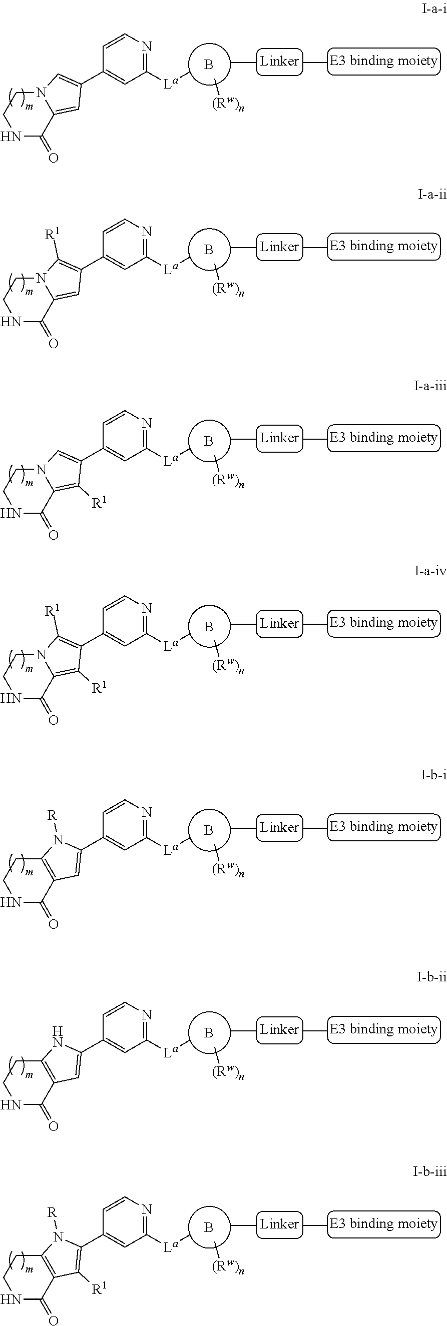

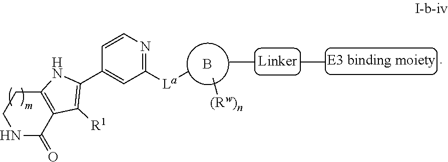

In some embodiments of the compounds of the present disclosure, the Linker is an optionally substituted bivalent C₂₋₂₀ straight or branched aliphatic chain, wherein one, two, three, four, or five methylene units of the aliphatic chain are optionally and independently replaced by a group selected from —N(R)—, —O—, —C(O)—, —N(R)C(O)—, —C(O)N(R)—, —OC(O)—, —C(O)O—, a bivalent 3- to 6-membered monocyclic saturated or partially unsaturated ring having 0-2 heteroatoms independently selected from nitrogen, oxygen, and sulfur, a bivalent 6- to 8-membered saturated or partially unsaturated bridged bicyclic, fused bicyclic or spirofused heterocyclic ring having 1-3 heteroatoms independently selected from nitrogen, oxygen, and sulfur, and phenylene, wherein each monocyclic ring, bridged bicyclic ring, fused bicyclic ring, spirofused ring, or phenylene is substituted by 0-4 instances of R^{L}; and
R^{L} is independently selected from halogen, —OR, —SR, —CN, —NO₂, —SO₂NR, —SO₂R, —SOR, —C(O)R, —CO₂R, —C(O)N(R)₂, —NRC(O)R, —NRC(O)OR, —NRC(O)N(R)₂, —NRSO₂R, —N(R)₂, or an optionally substituted group selected from the group consisting of C₁₋₆ aliphatic, phenyl, a 3- to 8-membered saturated or partially unsaturated carbocyclic ring, a 4- to 7-membered heterocyclic ring having 1-2 heteroatoms independently selected from nitrogen, oxygen, or sulfur, and a 5- to 6-membered monocyclic heteroaryl ring having 1-4 heteroatoms independently selected from nitrogen, oxygen, or sulfur.

In some embodiments of the compounds of the present disclosure, the Linker is an optionally substituted bivalent C₂₋₂₀ straight or branched aliphatic chain, wherein two, three, or four methylene units of the aliphatic chain are optionally and independently replaced by a group selected from —N(R)—, —O—, —C(O)—, —N(R)C(O)—, —C(O)N(R)—, —OC(O)—, —C(O)O—, and a bivalent 3- to 6-membered monocyclic saturated or partially unsaturated ring having 0-2 heteroatoms independently selected from nitrogen, oxygen, and sulfur, wherein the monocyclic ring is substituted by 0-4 instances of R^{L}.

In some embodiments of the compounds of the present disclosure, the Linker is an optionally substituted bivalent C₂₋₂₀ straight or branched aliphatic chain, wherein two, three, four, or five methylene units of the aliphatic chain are optionally and independently replaced by a group selected from —C(O)N(R)—, —N(R)—, —C(O)—,

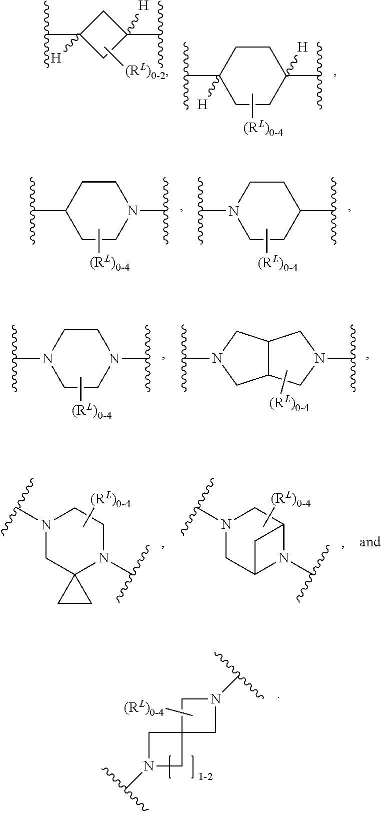

In some embodiments of the compounds of the present disclosure, the Linker is an optionally substituted bivalent C₂₋₂₀ straight or branched aliphatic chain, wherein two, three, four, or five methylene units of the aliphatic chain are optionally and independently replaced by a group selected from —C(O)N(R)—, —N(R)—, —C(O)—,

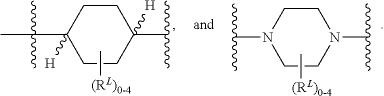

In some embodiments of the compounds of the present disclosure, the Linker is an optionally substituted bivalent C₃₋₁₇ straight or branched aliphatic chain, wherein one, two, three, four, or five methylene units of the aliphatic chain are optionally and independently replaced by a group selected from —N(R)—, —O—, —C(O)—, —N(R)C(O)—, —C(O)N(R)—, —OC(O)—, —C(O)O—, a bivalent 3- to 6-membered monocyclic saturated or partially unsaturated ring having 0-2 heteroatoms independently selected from nitrogen, oxygen, and sulfur, a bivalent 6- to 8-membered saturated or partially unsaturated bridged bicyclic, fused bicyclic or spirofused heterocyclic ring having 1-3 heteroatoms independently selected from nitrogen, oxygen, and sulfur, and phenylene, wherein each monocyclic ring, bridged bicyclic ring, fused bicyclic ring, spirofused ring, or phenylene is substituted by 0-4 instances of R^{L}.

In some embodiments of the compounds of the present disclosure, the Linker is an optionally substituted bivalent C₃₋₁₇ straight or branched aliphatic chain, wherein two, three, or four methylene units of the aliphatic chain are optionally and independently replaced by a group selected from —N(R)—, —O—, —C(O)—, —N(R)C(O)—, —C(O)N(R)—, —OC(O)—, —C(O)O—, and a bivalent 3- to 6-membered monocyclic saturated or partially unsaturated ring having 0-2 heteroatoms independently selected from nitrogen, oxygen, and sulfur, wherein the monocyclic ring is substituted by 0-4 instances of R^{L}.

In some embodiments of the compounds of the present disclosure, the Linker is an optionally substituted bivalent C₃₋₁₇ straight or branched aliphatic chain, wherein two, three, four, or five methylene units of the aliphatic chain are optionally and independently replaced by a group selected from —C(O)N(R)—, —N(R)—, —C(O)—,

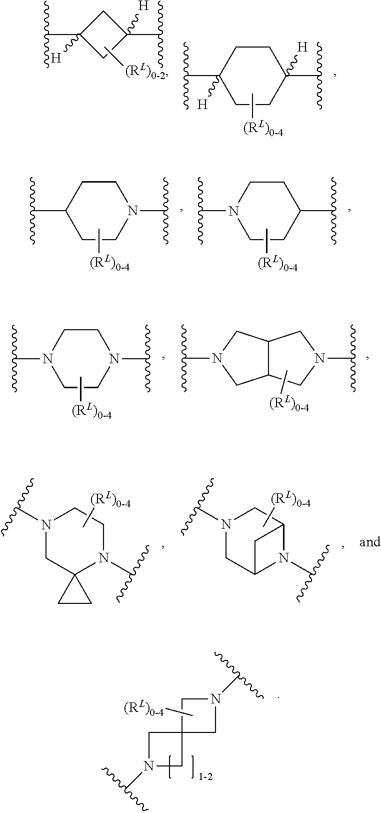

In some embodiments of the compounds of the present disclosure, the Linker is an optionally substituted bivalent C₃₋₁₇ straight or branched aliphatic chain, wherein two, three, four, or five methylene units of the aliphatic chain are optionally and independently replaced by a group selected from —C(O)N(R)—, —N(R)—, —C(O)—,

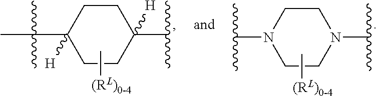

In some embodiments of the compounds of the present disclosure, the Linker is an optionally substituted bivalent C₂₋₁₀ straight or branched aliphatic chain, wherein one, two, three, four, or five methylene units of the aliphatic chain are optionally and independently replaced by a group selected from —N(R)—, —O—, —C(O)—, —N(R)C(O)—, —C(O)N(R)—, —OC(O)—, —C(O)O—, a bivalent 3- to 6-membered monocyclic saturated or partially unsaturated ring having 0-2 heteroatoms independently selected from nitrogen, oxygen, and sulfur, a bivalent 6- to 8-membered saturated or partially unsaturated bridged bicyclic, fused bicyclic or spirofused heterocyclic ring having 1-3 heteroatoms independently selected from nitrogen, oxygen, and sulfur, and phenylene, wherein each monocyclic ring, bridged bicyclic ring, fused bicyclic ring, spirofused ring, or phenylene is substituted by 0-4 instances of R^{L}.

In some embodiments of the compounds of the present disclosure, the Linker is an optionally substituted bivalent C₂₋₁₀ straight or branched aliphatic chain, wherein two, three, or four methylene units of the aliphatic chain are optionally and independently replaced by a group selected from —N(R)—, —O—, —C(O)—, —N(R)C(O)—, —C(O)N(R)—, —OC(O)—, —C(O)O—, and a bivalent 3- to 6-membered monocyclic saturated or partially unsaturated ring having 0-2 heteroatoms independently selected from nitrogen, oxygen, and sulfur, wherein the monocyclic ring is substituted by 0-4 instances of R^{L}.

In some embodiments of the compounds of the present disclosure, the Linker is an optionally substituted bivalent C₂₋₁₀ straight or branched aliphatic chain, wherein two, three, four, or five methylene units of the aliphatic chain are optionally and independently replaced by a group selected from —C(O)N(R)—, —N(R)—, —C(O)—,

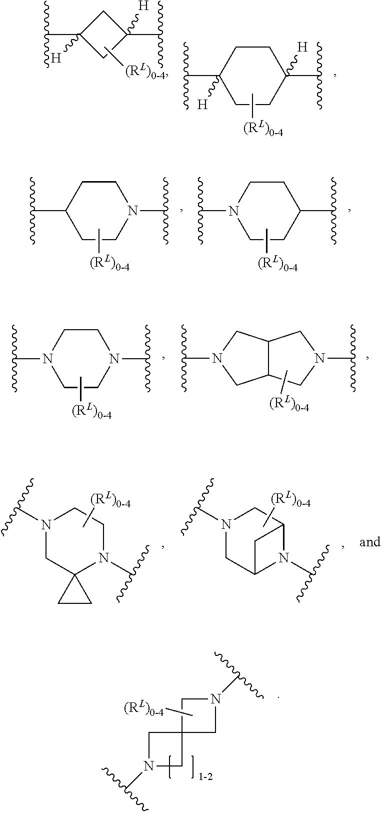

In some embodiments of the compounds of the present disclosure, the Linker is an optionally substituted bivalent C₂₋₁₀ straight or branched aliphatic chain, wherein two, three, four, or five methylene units of the aliphatic chain are optionally and independently replaced by a group selected from —C(O)N(R)—, —N(R)—, —C(O)—,

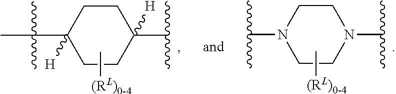

In some embodiments of the compounds of the present disclosure, the Linker is an optionally substituted bivalent C₂₋₆ straight or branched aliphatic chain, wherein one, two, three, four, or five methylene units of the aliphatic chain are optionally and independently replaced by a group selected from —N(R)—, —O—, —C(O)—, —N(R)C(O)—, —C(O)N(R)—, —OC(O)—, —C(O)O—, a bivalent 3- to 6-membered monocyclic saturated or partially unsaturated ring having 0-2 heteroatoms independently selected from nitrogen, oxygen, and sulfur, a bivalent 6- to 8-membered saturated or partially unsaturated bridged bicyclic, fused bicyclic or spirofused heterocyclic ring having 1-3 heteroatoms independently selected from nitrogen, oxygen, and sulfur, and phenylene, wherein each monocyclic ring, bridged bicyclic ring, fused bicyclic ring, spirofused ring, or phenylene is substituted by 0-4 instances of R^{L}.

In some embodiments of the compounds of the present disclosure, the Linker is an optionally substituted bivalent C₂₋₆ straight or branched aliphatic chain, wherein two, three, or four methylene units of the aliphatic chain are optionally and independently replaced by a group selected from —N(R)—, —O—, —C(O)—, —N(R)C(O)—, —C(O)N(R)—, —OC(O)—, —C(O)O—, and a bivalent 3- to 6-membered monocyclic saturated or partially unsaturated ring having 0-2 heteroatoms independently selected from nitrogen, oxygen, and sulfur, wherein the monocyclic ring is substituted by 0-4 instances of R^{L}.

In some embodiments of the compounds of the present disclosure, the Linker is an optionally substituted bivalent C₂₋₆ straight or branched aliphatic chain, wherein two, three, four, or five methylene units of the aliphatic chain are optionally and independently replaced by a group selected from —C(O)N(R)—, —N(R)—, —C(O)—,

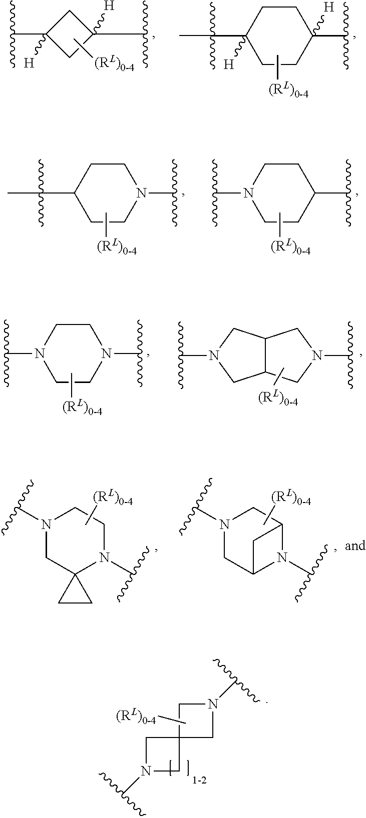

In some embodiments of the compounds of the present disclosure, the Linker is an optionally substituted bivalent C₂₋₆ straight or branched aliphatic chain, wherein two, three, four, or five methylene units of the aliphatic chain are optionally and independently replaced by a group selected from —C(O)N(R)—, —N(R)—, —C(O)—,

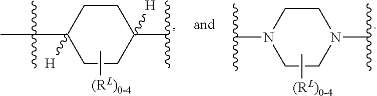

In some embodiments of the compounds of the present disclosure, the Linker is an optionally substituted bivalent C₃₋₆ straight or branched aliphatic chain, wherein one, two, three, four, or five methylene units of the aliphatic chain are optionally and independently replaced by a group selected from —N(R)—, —O—, —C(O)—, —N(R)C(O)—, —C(O)N(R)—, —OC(O)—, —C(O)O—, a bivalent 3- to 6-membered monocyclic saturated or partially unsaturated ring having 0-2 heteroatoms independently selected from nitrogen, oxygen, and sulfur, a bivalent 6- to 8-membered saturated or partially unsaturated bridged bicyclic, fused bicyclic or spirofused heterocyclic ring having 1-3 heteroatoms independently selected from nitrogen, oxygen, and sulfur, and phenylene, wherein each monocyclic ring, bridged bicyclic ring, fused bicyclic ring, spirofused ring, or phenylene is substituted by 0-4 instances of R^{L}.

In some embodiments of the compounds of the present disclosure, the Linker is an optionally substituted bivalent C₄₋₆ straight or branched aliphatic chain, wherein two, three, or four methylene units of the aliphatic chain are optionally and independently replaced by a group selected from —N(R)—, —O—, —C(O)—, —N(R)C(O)—, —C(O)N(R)—, —OC(O)—, —C(O)O—, and a bivalent 3- to 6-membered monocyclic saturated or partially unsaturated ring having 0-2 heteroatoms independently selected from nitrogen, oxygen, and sulfur, wherein the monocyclic ring is substituted by 0-4 instances of R^{L}.

In some embodiments of the compounds of the present disclosure, the Linker is an optionally substituted bivalent C₄₋₆ straight or branched aliphatic chain, wherein two, three, four, or five methylene units of the aliphatic chain are optionally and independently replaced by a group selected from —C(O)N(R)—, —N(R)—, —C(O)—,

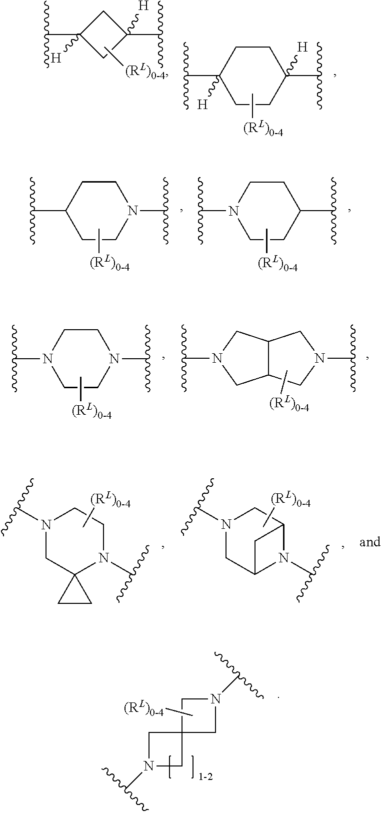

In some embodiments of the compounds of the present disclosure, the Linker is an optionally substituted bivalent C₄₋₆ straight or branched aliphatic chain, wherein two, three, four, or five methylene units of the aliphatic chain are optionally and independently replaced by a group selected from —C(O)N(R)—, —N(R)—, —CO—,

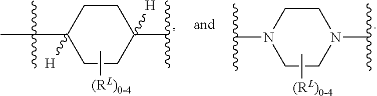

In some embodiments of the compounds of the present disclosure, the Linker is selected from the group consisting of:

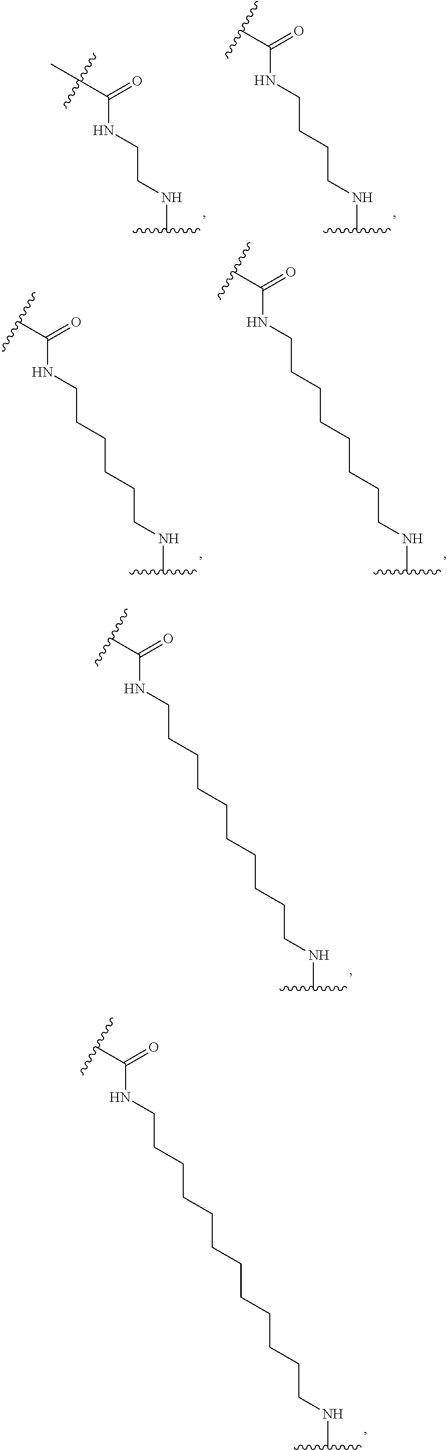 [SMILES: *NCCNC(*)=O ; *NCCCCNC(*)=O ; *NCCCCCCNC(*)=O ; *NCCCCCCCCNC(*)=O ; *NCCCCCCCCCCNC(*)=O ; *NCCCCCCCCCCCCNC(*)=O]

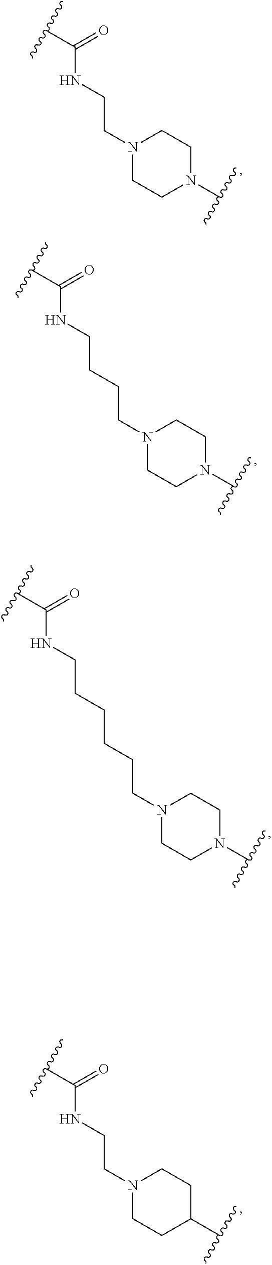 [SMILES: *C(=O)NCCN1CCN(*)CC1 ; *C(=O)NCCCCN1CCN(*)CC1 ; *C(=O)NCCCCCCN1CCN(*)CC1 ; *C(=O)NCCN1CCC(*)CC1]

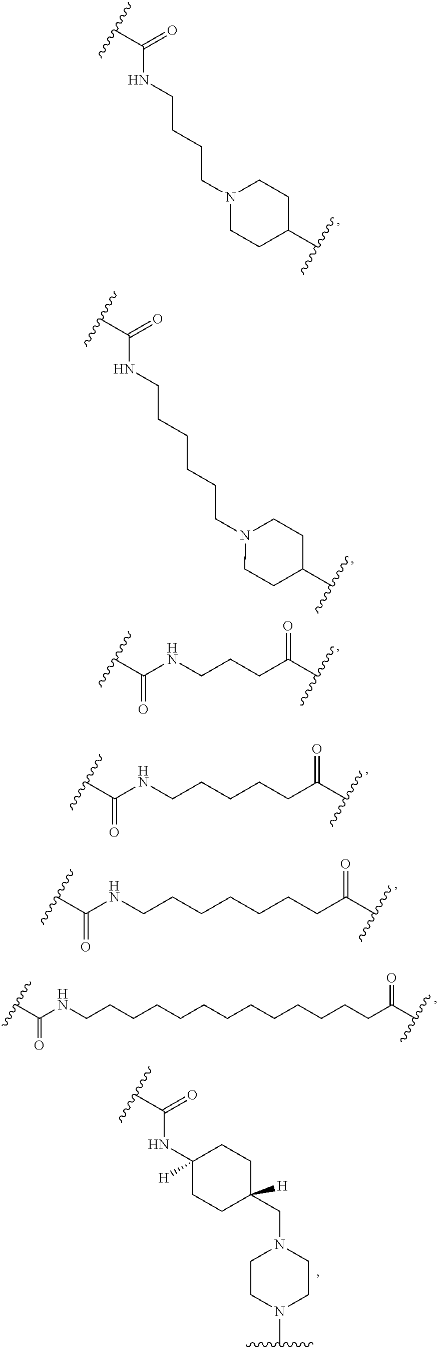

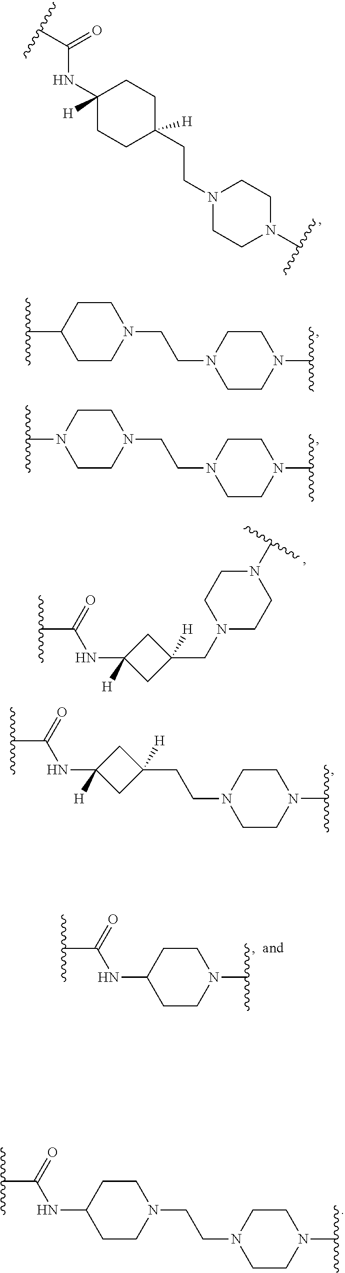

In some embodiments of the compounds of the present disclosure, the Linker is selected from the group consisting of:

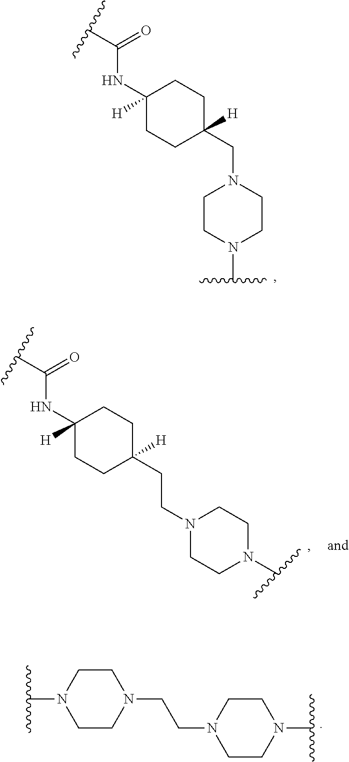 [SMILES: *C(=O)N[C@H]1CC[C@H](CN2CCN(*)CC2)CC1 ; *C(=O)N[C@H]1CC[C@H](CCN2CCN(*)CC2)CC1 ; *N1CCN(CCN2CCN(*)CC2)CC1]

In some embodiments of the compounds of the present disclosure, the E3 binding moiety is a cereblon protein binding moiety.

In some embodiments of the compounds of the present disclosure, the cereblon protein binding moiety is selected from:

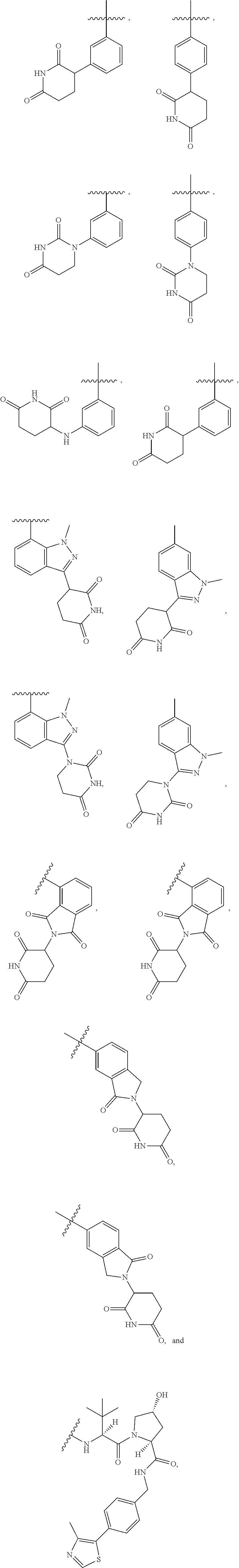

In some embodiments of the compounds of the present disclosure, the cereblon protein binding moiety is selected from:

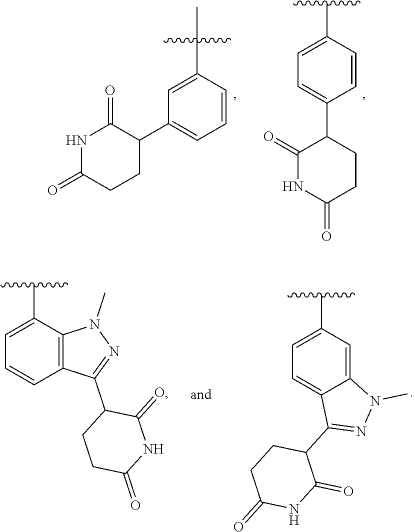

In some embodiments, the compound or pharmaceutically acceptable salt is selected from Table 1:

| | |
|---|---|
| 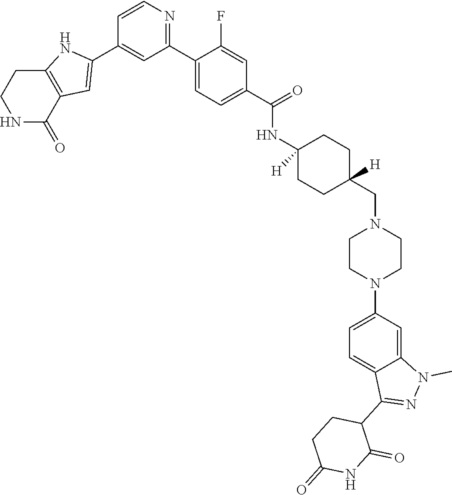 [SMILES: Cn1nc(C2CCC(=O)NC2=O)c2ccc(N3CCN(C[C@H]4CC[C@H](NC(=O)c5ccc(-c6cc(-c7cc8c([nH]7)CCNC8=O)ccn6)c(F)c5)CC4)CC3)cc21] | Ex. 1.5 |
| 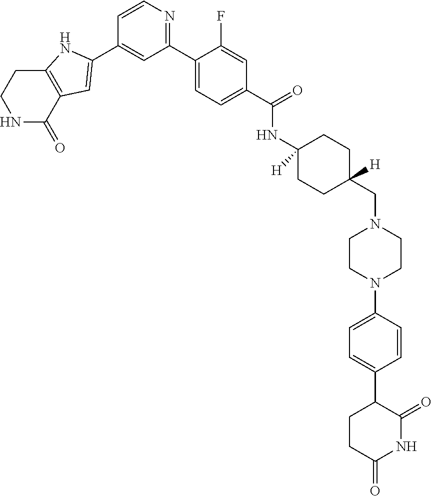 [SMILES: O=C1CCC(c2ccc(N3CCN(C[C@H]4CC[C@H](NC(=O)c5ccc(-c6cc(-c7cc8c([nH]7)CCNC8=O)ccn6)c(F)c5)CC4)CC3)cc2)C(=O)N1] | Ex 1.6 |
| 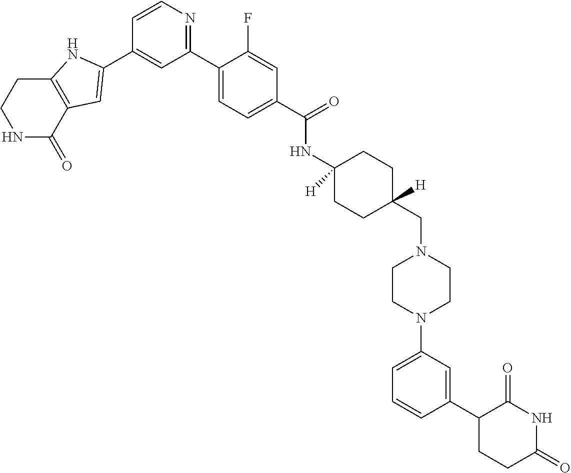 [SMILES: O=C1CCC(c2cccc(N3CCN(C[C@H]4CC[C@H](NC(=O)c5ccc(-c6cc(-c7cc8c([nH]7)CCNC8=O)ccn6)c(F)c5)CC4)CC3)c2)C(=O)N1] | Ex 1.7 |
| 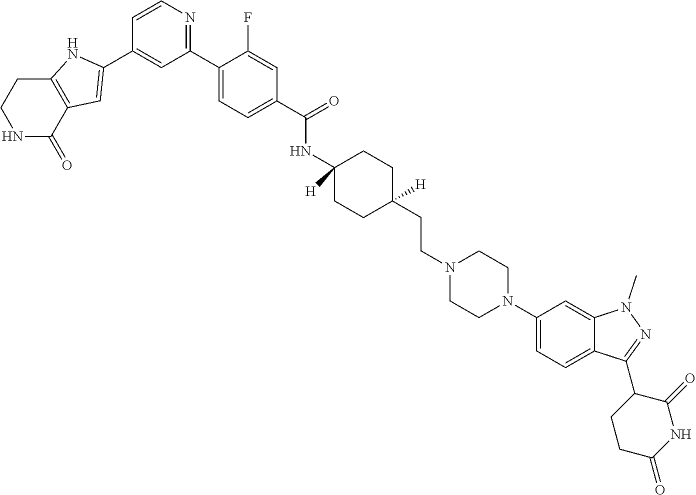 [SMILES: Cn1nc(C2CCC(=O)NC2=O)c2ccc(N3CCN(CC[C@H]4CC[C@H](NC(=O)c5ccc(-c6cc(-c7cc8c([nH]7)CCNC8=O)ccn6)c(F)c5)CC4)CC3)cc21] | Ex 1.8 |
| 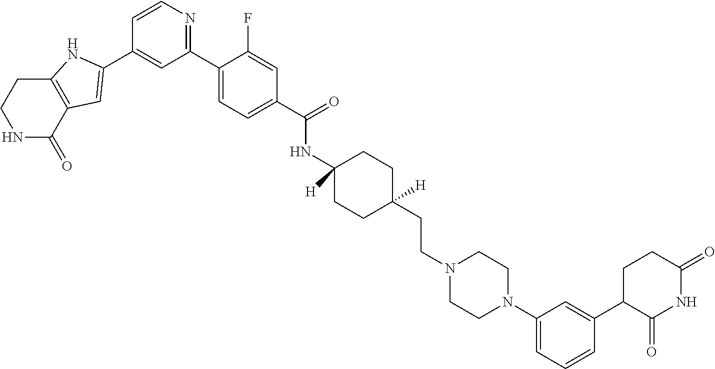 [SMILES: O=C1CCC(c2cccc(N3CCN(CC[C@H]4CC[C@H](NC(=O)c5ccc(-c6cc(-c7cc8c([nH]7)CCNC8=O)ccn6)c(F)c5)CC4)CC3)c2)C(=O)N1] | Ex 1.9 |
| 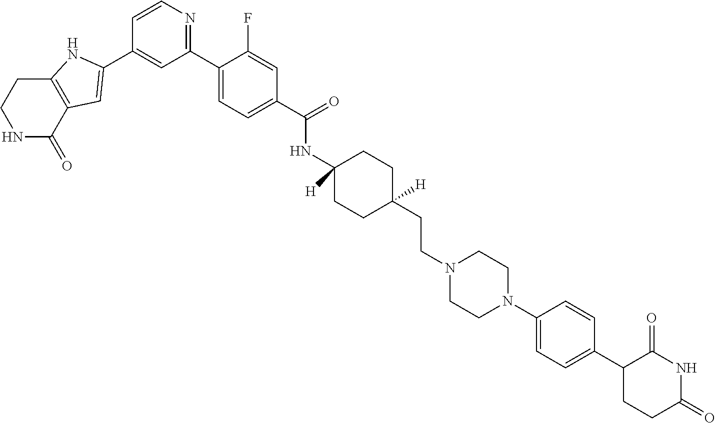 [SMILES: O=C1CCC(c2ccc(N3CCN(CC[C@H]4CC[C@H](NC(=O)c5ccc(-c6cc(-c7cc8c([nH]7)CCNC8=O)ccn6)c(F)c5)CC4)CC3)cc2)C(=O)N1] | Ex 1.10 |
| 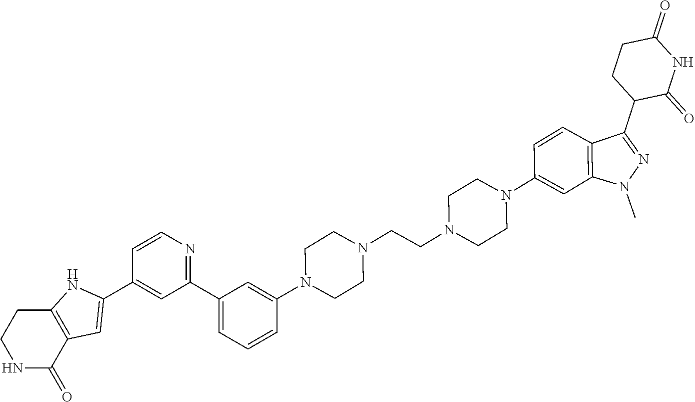 [SMILES: Cn1nc(C2CCC(=O)NC2=O)c2ccc(N3CCN(CCN4CCN(c5cccc(-c6cc(-c7cc8c([nH]7)CCNC8=O)ccn6)c5)CC4)CC3)cc21] | Ex 1.11 |
| 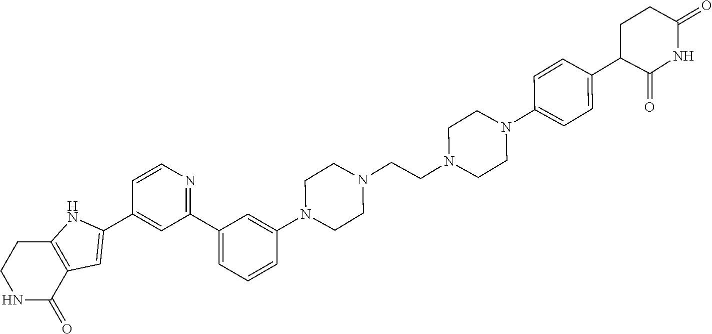 [SMILES: O=C1CCC(c2ccc(N3CCN(CCN4CCN(c5cccc(-c6cc(-c7cc8c([nH]7)CCNC8=O)ccn6)c5)CC4)CC3)cc2)C(=O)N1] | Ex 1.12 |
| 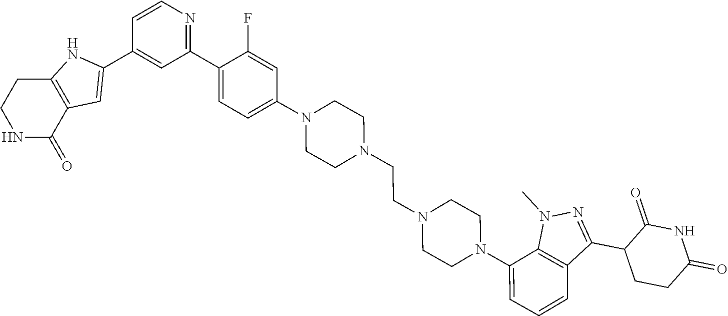 [SMILES: Cn1nc(C2CCC(=O)NC2=O)c2cccc(N3CCN(CCN4CCN(c5ccc(-c6cc(-c7cc8c([nH]7)CCNC8=O)ccn6)c(F)c5)CC4)CC3)c21] | Ex 1.13 |

In other embodiments of the disclosure, the pharmaceutical composition described herein comprises a compound described herein and a pharmaceutically acceptable excipient, carrier, or diluent.

In other embodiments of the disclosure, the method of degrading the activity of MK2, or a mutant thereof comprises contacting a biological sample with a compound of the present disclosure.

In another embodiment of the disclosure, the method of treating a disease, disorder, or condition mediated by MK2, or a mutant thereof comprises administering to a patient in need thereof a compound or a composition of the present disclosure.

### 4. Uses, Formulation and Administration

### Pharmaceutically Acceptable Compositions

According to another embodiment, the present disclosure provides a composition comprising a compound described herein, or a pharmaceutically acceptable salt thereof, and a pharmaceutically acceptable carrier, adjuvant, or vehicle. In certain embodiments, the amount of compound in provided compositions is sufficient to measurably degrade MK2, or a mutant thereof, in a biological sample or in a patient. In certain embodiments, a provided composition is formulated for administration to a patient in need of such composition. In some embodiments, a provided composition is formulated for oral administration to a patient.

Compounds and compositions, according to a provided method, are administered using any amount and any route of administration effective for treating or lessening the severity of a disorder provided herein (i.e., an MK2-mediated disease or disorder). The exact amount required will vary from subject to subject, depending on the species, age, and general condition of the subject, the severity of the infection, the particular agent, its mode of administration, and the like. Compounds described herein may further be formulated in unit dosage form for ease of administration and uniformity of dosage.

Compositions provided herein may be administered orally, parenterally, by inhalation spray, topically, rectally, nasally, buccally, vaginally, intraperitoneally, intracisternally or via an implanted reservoir. In some embodiments, the compositions are administered orally, intraperitoneally or intravenously.

Sterile injectable forms of the compositions provided herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound provided herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

In some embodiments, provided pharmaceutically acceptable compositions are formulated for oral administration. Such formulations may be administered with or without food. In some embodiments, pharmaceutically acceptable compositions provided herein are administered without food. In other embodiments, pharmaceutically acceptable compositions provided herein are administered with food. Pharmaceutically acceptable compositions provided herein may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Solid dosage forms for oral administration include capsules, tablets, pills, powders, and granules. In such solid dosage forms, a provided compound is mixed with at least one inert, pharmaceutically acceptable excipient or carrier such as (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and/or (i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only in a certain part of the intestinal tract, optionally, in a delayed manner.

Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms, a provided compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that releases the active ingredient(s) only in a certain part of the intestinal tract, optionally, in a delayed manner. Examples of embedding compositions that can be used include polymeric substances and waxes.

Liquid dosage forms for oral administration include, but are not limited to, pharmaceutically acceptable emulsions, microemulsions, solutions, suspensions, syrups and elixirs. In addition to the provided compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can also include adjuvants such as wetting agents, emulsifying and suspending agents, sweetening, flavoring, and perfuming agents.

Alternatively, pharmaceutically acceptable compositions provided herein may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Compositions for rectal or vaginal administration may also be suppositories which can be prepared by mixing the compounds provided herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Pharmaceutically acceptable compositions provided herein may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing a compound described herein suspended or dissolved in one or more carriers. Carriers for topical administration of provided compounds include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing a compound described herein suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, including solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions provided herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Dosage forms for topical or transdermal administration of a compound described herein include ointments, pastes, creams, lotions, gels, powders, solutions, sprays, inhalants or patches. A compound may be admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Uses of Compounds and Pharmaceutically Acceptable Compositions

Compounds and compositions described herein are generally useful for the degradation of kinase activity of one or more enzymes and the treatment of diseases and disorders associated with MK2 degradation. Examples of kinases that are degraded by the compounds and compositions described herein and against which the methods described herein are useful include MK2, or a mutant thereof.

The activity of a compound utilized as a degrader of a MK2 kinase, or a mutant thereof, may be assayed in vitro, in vivo or in a cell line. In vitro assays include assays that determine inhibition of either the phosphorylation activity and/or the subsequent functional consequences, or ATPase activity of activated MK2 kinase, or a mutant thereof. Alternate in vitro assays quantitate the ability of a provided compound to bind to MK2. Degrader activity may be measured by radiolabeling the test compound prior to binding, isolating the compound/MK2 complex and determining the amount of radiolabel bound. Alternatively, degrader activity may be determined by running a competition experiment where test compounds are incubated with MK2 kinase bound to known radioligands. Detailed conditions for assaying a compound utilized in this disclosure as a degrader of MK2, or a mutant thereof, are set forth in the Examples, below.

According to one embodiment, the present disclosure relates to a method of degrading protein kinase activity in a biological sample comprising the step of contacting said biological sample with a compound provided herein, or a composition comprising said compound.

According to another embodiment, the present disclosure relates to a method of degrading MK2 kinase, or a mutant thereof, activity in a biological sample comprising the step of contacting said biological sample with a compound provided herein, or a composition comprising said compound. In certain embodiments, the present disclosure relates to a method of irreversibly degrading MK2 kinase, or a mutant thereof, activity in a biological sample comprising the step of contacting said biological sample with a compound provided herein, or a composition comprising said compound.

According to another embodiment, the present disclosure relates to a method of degrading MK2 kinase, or a mutant thereof, activity in a patient comprising the step of administering to said patient a compound provided herein, or a composition comprising said compound. According to certain embodiments, the present disclosure relates to a method of irreversibly degrading MK2 kinase, or a mutant thereof, activity in a patient comprising the step of administering to said patient a compound provided herein, or a composition comprising said compound. In other embodiments, the present disclosure provides a method for treating an MK2-mediated disease or disorder, in a patient in need thereof, comprising the step of administering to said patient a compound provided herein or pharmaceutically acceptable composition thereof. Such disorders are described in detail herein.

### MK2 Kinase

MAP kinase-activated protein kinase 2 (“MK2”) is an enzyme that in humans is encoded by the MAPKAPK2 gene. This gene encodes a member of the Ser/Thr protein kinase family. This kinase is regulated through direct phosphorylation by p38 MAP kinase. In conjunction with p38 MAP kinase, this kinase is known to be involved in many cellular processes including stress and inflammatory responses, nuclear export, gene expression regulation and cell proliferation. Heat shock protein HSP27 was shown to be one of the substrates of this kinase in vivo. Two transcript variants encoding two different isoforms have been found for this gene.

MK2 is a multi-domain protein consisting of an N-terminal proline-rich domain, a catalytic domain, an autoinhibitory domain and at the C-terminus a nuclear export signal (NES) and nuclear localization signal (NLS). Two isoforms of human MK2 have been characterized. One isoform consists of 400 amino acids and the other isoform 370 residues which is thought to be a splice variant missing the C-terminal NLS. MK2 is located in the nucleus of the cell and upon binding and phosphorylation by p38, the MK2 NES becomes functional and both kinases are co-transported out of the nucleus to the cytoplasm. Interestingly, transport of the MK2/p38 complex does not require catalytically active MK2, as the active site mutant, Asp207Ala, is still transported to the cytoplasm. Phosphorylation of human MK2 by p38 on residues T222, S272 and T334 is thought to activate the enzyme by inducing a conformational change of the autoinhibitory domain thus exposing the active site for substrate binding. Mutations of two autoinhibitory domain residues W332A and K326E in murine MK2 demonstrate an increase in basal activity and a C-terminal deletion of the autoinhibitory domain renders the enzyme constitutively active, providing additional evidence to the role of this domain in inhibition of MK2 activity.

Diseases or disorders associated with MK2 that are treated by compounds of the present disclosure include autoimmune disorders, chronic inflammatory disorders, acute inflammatory disorders, auto-inflammatory disorders, fibrotic disorders, metabolic disorders, neoplasias, or cardiovascular or cerebrovascular disorders. Thus, in some embodiments, the present disclosure provides a method for treating an MK2-mediated disease or disorder in a patient in need thereof, wherein said method comprises administering to said patient a therapeutically effective amount of a provided compound, or composition thereof. Such MK2-mediated diseases or disorders include, but are not limited to those described herein.

In some embodiments, the MK2-mediated disease or disorder is an autoimmune disorder, chronic and/or acute inflammatory disorder, and/or auto-inflammatory disorder. Exemplary autoimmune and/or inflammatory and/or auto-inflammatory disorders include: inflammatory bowel diseases (for example, ulcerative colitis or Crohn's disease), multiple sclerosis, psoriasis, arthritis, rheumatoid arthritis, osteoarthritis, juvenile arthritis, psoriatic arthritis, reactive arthritis, ankylosing spondylitis, cryopyrin associated periodic syndromes, Muckle-Wells syndrome, familial cold auto-inflammatory syndrome, neonatal-onset multisystem inflammatory disease, TNF receptor associated periodic syndrome, acute and chronic pancreatitis, atherosclerosis, gout, ankylosing spondylitis, fibrotic disorders (for example, hepatic fibrosis or idiopathic pulmonary fibrosis), nephropathy, sarcoidosis, scleroderma, anaphylaxis, diabetes (for example, diabetes mellitus type 1 or diabetes mellitus type 2), diabetic retinopathy, Still's disease, vasculitis, sarcoidosis, pulmonary inflammation, acute respiratory distress syndrome, wet and dry age-related macular degeneration, autoimmune hemolytic syndromes, autoimmune and inflammatory hepatitis, autoimmune neuropathy, autoimmune ovarian failure, autoimmune orchitis, autoimmune thrombocytopenia, silicone implant associated autoimmune disease, Sjogren's syndrome, familial Mediterranean fever, systemic lupus erythematosus, vasculitis syndromes (for example, temporal, Takayasu's and giant cell arteritis, Behçet's disease or Wegener's granulomatosis), vitiligo, secondary hematologic manifestation of autoimmune diseases (for example, anemias), drug-induced autoimmunity, Hashimoto's thyroiditis, hypophysitis, idiopathic thrombocytic pupura, metal-induced autoimmunity, myasthenia gravis, pemphigus, autoimmune deafness (for example, Meniere's disease), Goodpasture's syndrome, Graves' disease, HW-related autoimmune syndromes, Gullain-Barre disease, Addison's disease, anti-phospholipid syndrome, asthma, atopic dermatitis, Celiac disease, Cushing's syndrome, dermatomyositis, idiopathic adrenal atrophy, idiopathic thrombocytopenia, Kawasaki syndrome, Lambert-Eaton Syndrome, pernicious anemia, pollinosis, polyarteritis nodosa, primary biliary cirrhosis, primary sclerosing cholangitis, Raynaud's, Reiter's Syndrome, relapsing polychondritis, Schmidt's syndrome, thyrotoxidosis, sepsis, septic shock, endotoxic shock, exotoxin-induced toxic shock, gram negative sepsis, toxic shock syndrome, glomerulonephritis, peritonitis, interstitial cystitis, hyperoxia-induced inflammations, chronic obstructive pulmonary disease (COPD), vasculitis, graft vs. host reaction (for example, graft vs. host disease), allograft rejections (for example, acute allograft rejection or chronic allograft rejection), early transplantation rejection (for example, acute allograft rejection), reperfusion injury, pain (for example, acute pain, chronic pain, neuropathic pain, or fibromyalgia), chronic infections, meningitis, encephalitis, myocarditis, gingivitis, post surgical trauma, tissue injury, traumatic brain injury, enterocolitis, sinusitis, uveitis, ocular inflammation, optic neuritis, gastric ulcers, esophagitis, peritonitis, periodontitis, dermatomyositis, gastritis, myositis, polymyalgia, pneumonia and bronchitis.

In some embodiments, the MK2-mediated disease or disorder is a fibrotic disorder. Exemplary fibrotic disorders include systemic sclerosis/scleroderma, lupus nephritis, connective tissue disease, wound healing, surgical scarring, spinal cord injury, CNS scarring, acute lung injury, pulmonary fibrosis (for example, idiopathic pulmonary fibrosis or cystic fibrosis), chronic obstructive pulmonary disease, adult respiratory distress syndrome, acute lung injury, drug-induced lung injury, glomerulonephritis, chronic kidney disease (for example, diabetic nephropathy), hypertension-induced nephropathy, alimentary track or gastrointestinal fibrosis, renal fibrosis, hepatic or biliary fibrosis, liver fibrosis (for example, nonalcoholic steatohepatitis, hepatitis C, or hepatocellular carcinoma), cirrhosis (for example, primary biliary cirrhosis or cirrhosis due to fatty liver disease (for example, alcoholic and nonalcoholic steatosis)), radiation-induced fibrosis (for example, head and neck, gastrointestinal or pulmonary), primary sclerosing cholangitis, restenosis, cardiac fibrosis (for example, endomyocardial fibrosis or atrial fibrosis), ophthalmic scarring, fibrosclerosis, fibrotic cancers, fibroids, fibroma, fibroadenomas, fibrosarcomas, transplant arteriopathy, keloid, mediastinal fibrosis, myelofibrosis, retroperitoneal fibrosis, progressive massive fibrosis, and nephrogenic systemic fibrosis.

In some embodiments, the MK2-mediated disease or disorder is a metabolic disorder. Exemplary metabolic disorders include obesity, steroid-resistance, glucose intolerance, and metabolic syndrome.

In some embodiments, the MK2-mediated disease or disorder is a neoplasia. Exemplary neoplasias include cancers. In some embodiments, exemplary neoplasias include angiogenesis disorders, multiple myeloma, leukemias (for example, acute lymphocytic leukemia, acute and chronic myelogenous leukemia, chronic lymphocytic leukemia, acute lymphoblastic leukemia, or promyelocytic leukemia), lymphomas (for example, B-cell lymphoma, T-cell lymphoma, mantle cell lymphoma, hairy cell lymphoma, Burkitt's lymphoma, mast cell tumors, Hodgkin's disease or non-Hodgkin's disease), myelodysplastic syndrome, fibrosarcoma, rhabdomyosarcoma; astrocytoma, neuroblastoma, glioma and schwannomas; melanoma, seminoma, teratocarcinoma, osteosarcoma, xenoderma pigmentosum, keratoctanthoma, thyroid follicular cancer, Kaposi's sarcoma, melanoma, teratoma, rhabdomyosarcoma, metastatic and bone disorders, as well as cancer of the bone, mouth/pharynx, esophagus, larynx, stomach, intestine, colon, rectum, lung (for example, non-small cell lung cancer or small cell lung cancer), liver, pancreas, nerve, brain (for example, glioma or glioblastoma multiforme), head and neck, throat, ovary, uterus, prostate, testis, bladder, kidney, breast, gall bladder, cervix, thyroid, prostate, and skin.

In some embodiments, the MK2-mediated disorder is a cardiovascular or cerebrovascular disorder. Exemplary cardiovascular disorders include atherosclerosis, restenosis of an atherosclerotic coronary artery, acute coronary syndrome, myocardial infarction, cardiac-allograft vasculopathy and stroke. Exemplary cerebrovascular diseases include central nervous system disorders with an inflammatory or apoptotic component, Alzheimer's disease, Parkinson's disease, Huntington's disease, amyotrophic lateral sclerosis, spinal cord injury, neuronal ischemia and peripheral neuropathy.

Diseases or disorders associated with MK2 that are treated by a compound provided herein include autoimmune disorders, chronic inflammatory disorders, acute inflammatory disorders, auto-inflammatory disorders, fibrotic disorders, metabolic disorders, neoplasias, or cardiovascular or cerebrovascular disorders. Thus, in some embodiments, the present disclosure provides a method for treating an MK2-mediated disease or disorder in a patient in need thereof, wherein said method comprises administering to said patient a composition comprising a therapeutically effective amount of a compound provided herein. Such MK2-mediated diseases or disorders include, but are not limited to those described herein.

In some embodiments, the MK2-mediated disease or disorder is an autoimmune disorder, chronic and/or acute inflammatory disorder, and/or auto-inflammatory disorder. Exemplary autoimmune and/or inflammatory and/or auto-inflammatory disorders include: inflammatory bowel diseases (for example, ulcerative colitis or Crohn's disease), multiple sclerosis, psoriasis, arthritis, rheumatoid arthritis, osteoarthritis, juvenile arthritis, psoriatic arthritis, reactive arthritis, ankylosing spondylitis, cryopyrin associated periodic syndromes, Muckle-Wells syndrome, familial cold auto-inflammatory syndrome, neonatal-onset multisystem inflammatory disease, TNF receptor associated periodic syndrome, acute and chronic pancreatitis, atherosclerosis, gout, ankylosing spondylitis, fibrotic disorders (for example, hepatic fibrosis or idiopathic pulmonary fibrosis), nephropathy, sarcoidosis, scleroderma, anaphylaxis, diabetes (for example, diabetes mellitus type 1 or diabetes mellitus type 2), diabetic retinopathy, Still's disease, vasculitis, sarcoidosis, pulmonary inflammation, acute respiratory distress syndrome, wet and dry age-related macular degeneration, autoimmune hemolytic syndromes, autoimmune and inflammatory hepatitis, autoimmune neuropathy, autoimmune ovarian failure, autoimmune orchitis, autoimmune thrombocytopenia, silicone implant associated autoimmune disease, Sjogren's syndrome, familial Mediterranean fever, systemic lupus erythematosus, vasculitis syndromes (for example, temporal, Takayasu's and giant cell arteritis, Behçet's disease or Wegener's granulomatosis), vitiligo, secondary hematologic manifestation of autoimmune diseases (for example, anemias), drug-induced autoimmunity, Hashimoto's thyroiditis, hypophysitis, idiopathic thrombocytic pupura, metal-induced autoimmunity, myasthenia gravis, pemphigus, autoimmune deafness (for example, Meniere's disease), Goodpasture's syndrome, Graves' disease, HW-related autoimmune syndromes, Gullain-Barre disease, Addison's disease, anti-phospholipid syndrome, asthma, atopic dermatitis, Celiac disease, Cushing's syndrome, dermatomyositis, idiopathic adrenal atrophy, idiopathic thrombocytopenia, Kawasaki syndrome, Lambert-Eaton Syndrome, pernicious anemia, pollinosis, polyarteritis nodosa, primary biliary cirrhosis, primary sclerosing cholangitis, Raynaud's, Reiter's Syndrome, relapsing polychondritis, Schmidt's syndrome, thyrotoxidosis, sepsis, septic shock, endotoxic shock, exotoxin-induced toxic shock, gram negative sepsis, toxic shock syndrome, glomerulonephritis, peritonitis, interstitial cystitis, hyperoxia-induced inflammations, chronic obstructive pulmonary disease (COPD), vasculitis, graft vs. host reaction (for example, graft vs. host disease), allograft rejections (for example, acute allograft rejection or chronic allograft rejection), early transplantation rejection (for example, acute allograft rejection), reperfusion injury, pain (for example, acute pain, chronic pain, neuropathic pain, or fibromyalgia), chronic infections, meningitis, encephalitis, myocarditis, gingivitis, post-surgical trauma, tissue injury, traumatic brain injury, enterocolitis, sinusitis, uveitis, ocular inflammation, optic neuritis, gastric ulcers, esophagitis, peritonitis, periodontitis, dermatomyositis, gastritis, myositis, polymyalgia, pneumonia and bronchitis.

In some embodiments, the MK2-mediated disease or disorder is a fibrotic disorder. Exemplary fibrotic disorders include systemic sclerosis/scleroderma, lupus nephritis, connective tissue disease, wound healing, surgical scarring, spinal cord injury, CNS scarring, acute lung injury, pulmonary fibrosis (for example, idiopathic pulmonary fibrosis or cystic fibrosis), chronic obstructive pulmonary disease, adult respiratory distress syndrome, acute lung injury, drug-induced lung injury, glomerulonephritis, chronic kidney disease (for example, diabetic nephropathy), hypertension-induced nephropathy, alimentary track or gastrointestinal fibrosis, renal fibrosis, hepatic or biliary fibrosis, liver fibrosis (for example, nonalcoholic steatohepatitis, hepatitis C, or hepatocellular carcinoma), cirrhosis (for example, primary biliary cirrhosis or cirrhosis due to fatty liver disease (for example, alcoholic and nonalcoholic steatosis)), radiation-induced fibrosis (for example, head and neck, gastrointestinal or pulmonary), primary sclerosing cholangitis, restenosis, cardiac fibrosis (for example, endomyocardial fibrosis or atrial fibrosis), ophthalmic scarring, fibrosclerosis, fibrotic cancers, fibroids, fibroma, fibroadenomas, fibrosarcomas, transplant arteriopathy, keloid, mediastinal fibrosis, myelofibrosis, retroperitoneal fibrosis, progressive massive fibrosis, and nephrogenic systemic fibrosis.

In some embodiments, the MK2-mediated disease or disorder is a metabolic disorder. Exemplary metabolic disorders include obesity, steroid-resistance, glucose intolerance, and metabolic syndrome.

In some embodiments, the MK2-mediated disease or disorder is a neoplasia. Exemplary neoplasias include cancers. In some embodiments, exemplary neoplasias include angiogenesis disorders, multiple myeloma, leukemias (for example, acute lymphocytic leukemia, acute and chronic myelogenous leukemia, chronic lymphocytic leukemia, acute lymphoblastic leukemia, or promyelocytic leukemia), lymphomas (for example, B-cell lymphoma, T-cell lymphoma, mantle cell lymphoma, hairy cell lymphoma, Burkitt's lymphoma, mast cell tumors, Hodgkin's disease or non-Hodgkin's disease), myelodysplastic syndrome, fibrosarcoma, rhabdomyosarcoma; astrocytoma, neuroblastoma, glioma and schwannomas; melanoma, seminoma, teratocarcinoma, osteosarcoma, xenoderma pigmentosum, keratoctanthoma, thyroid follicular cancer, Kaposi's sarcoma, melanoma, teratoma, rhabdomyosarcoma, metastatic and bone disorders, as well as cancer of the bone, mouth/pharynx, esophagus, larynx, stomach, intestine, colon, rectum, lung (for example, non-small cell lung cancer or small cell lung cancer), liver, pancreas, nerve, brain (for example, glioma or glioblastoma multiforme), head and neck, throat, ovary, uterus, prostate, testis, bladder, kidney, breast, gall bladder, cervix, thyroid, prostate, and skin.

In some embodiments, the MK2-mediated disorder is a cardiovascular or cerebrovascular disorder. Exemplary cardiovascular disorders include atherosclerosis, restenosis of an atherosclerotic coronary artery, acute coronary syndrome, myocardial infarction, cardiac-allograft vasculopathy and stroke. Exemplary cerebrovascular diseases include central nervous system disorders with an inflammatory or apoptotic component, Alzheimer's disease, Parkinson's disease, Huntington's disease, amyotrophic lateral sclerosis, spinal cord injury, neuronal ischemia and peripheral neuropathy.

Accordingly, in some embodiments, provided formulations may be used to treat a MK2-mediated disease or disorder. In certain embodiments, the present disclosure provides methods of administering a provided formulation to human subjects.

Administration of provided formulations may be advantageous for the treatment, stabilization or lessening the severity or progression of one or more diseases and conditions associated with MK2 comprising the step of administering to the subject a provided formulation, as described herein. Such disorders or conditions include, among others, autoimmune diseases, inflammatory diseases, bone diseases, metabolic diseases, neurological and neurodegenerative diseases, cancer, cardiovascular diseases, allergies and asthma, Alzheimer's disease, and hormone-related diseases.

In some embodiments, the disease or disorder associated with MK2 is an autoimmune disease or disorder. In some embodiments, the disease or disorder associated with MK2 is an inflammatory disease or disorder. In some such embodiments, the inflammatory disease or disorder is selected from a chronic inflammatory disorder, an acute inflammatory disorder, or an auto-inflammatory disorder. In some embodiments, such autoimmune or inflammatory diseases and disorders are selected from rheumatoid arthritis, psoriatic arthritis, psoriasis, and ankylosing spondylitis.

In some embodiments, the present disclosure provides a method of preventing the progression of an autoimmune or inflammatory disease or disorder associated with MK2, comprising administering to a patient in need thereof a composition comprising a therapeutically effective amount of a compound provided herein. In some embodiments, such autoimmune or inflammatory diseases and disorders are selected from rheumatoid arthritis, psoriatic arthritis, psoriasis, and ankylosing spondylitis.

### Ankylosing Spondylitis

Ankylosing spondylitis (AS) is a chronic form of arthritis that primarily affects the spine, although other joints can become involved. A systemic inflammatory disease of indeterminate etiology, ankylosing spondylitis affects the axial spine (spondylitis), with sacroiliitis as its hallmark. The most common presenting symptom is chronic back pain and progressive spinal stiffness, a result of inflammation affecting the spine and sacroiliac joints (Feld et al. Axial disease in psoriatic arthritis and ankylosing spondylitis: a critical comparison. Nat Rev Rheumatol 2018; 14(6):363-71). In more advanced cases this inflammation can lead to ankylosis—new bone formation in the spine—causing sections of the spine to fuse in a fixed, immobile position.

Ankylosing spondylitis can also cause inflammation, pain, and stiffness in other areas of the body such as the shoulders, hips, ribs, heels, and small joints of the hands and feet. Sometimes the eyes can become involved (known as iritis or uveitis), and—rarely—the lungs and heart can be affected. The hallmark feature of ankylosing spondylitis is the involvement of the sacroiliac (SI) joints during the progression of the disease. The SI joints are located at the base of the spine, where the spine joins the pelvis.

Ankylosing spondylitis is typically diagnosed in people younger than 40 years and about 80% of patients develop first symptoms when they are younger than 30 years (Hanson et al. Genetics and the Causes of Ankylosing Spondylitis. Rheum Dis Clin North Am. 2017; 43(3):401-14). It is estimated that approximately 70% of patients with AS are males (de Winter et al. Prevalence of peripheral and extra-articular disease in ankylosing spondylitis versus non-radiographic axial spondyloarthritis: a meta-analysis. Arthritis Res Ther 2016; 18:196) Recent studies reported the prevalence of AS to range from 9 to 30 per 10,000 in the general population, depending on geographic area, study population or data source, case definition, and ascertainment methods. In general, there is a clear correlation between the prevalence of AS in a given population and the prevalence of HLA-B27 in that group, with the prevalence of AS being approximately 5 to 6 percent among people who are HLA-B27-positive (Reveille et al. The Epidemiology of Back Pain, Axial Spondyloarthritis and HLA-B27 in the United States. Am J Med Sci. 2013; 345(6): 431-6). Approximately 94% of individuals with AS are HLA-B27-positive (Brown et al. HLA class I associations of ankylosing spondylitis in the white population in the United Kingdom. Ann Rheum Dis. 1996; 55(4):268-70).

Although HLA-B27 is the largest single genetic contributor to disease pathophysiology, many other genetic loci, including those associated with the interleukin (IL)-17A pathway, have been associated with AS (Brown et al. Genetics of ankylosing spondylitis—insights into pathogenesis. Nat Rev Rheumatol. 2016; 12(2):81-91; Costantino et al. Genetics and Functional Genomics of Spondyloarthritis. Front Immunol. 2018:9:2933). Chronic inflammation in AS is thought to be driven by CD4+ and/or CD8+ T lymphocytes, including innate-like lymphocytes, and cytokines such as tumor necrosis factor (TNF)-α and IL-17A (Ranganathan et al. Macrophage Migration Inhibitory Factor Induces Inflammation and Predicts Spinal Progression in Ankylosing Spondylitis. Arthritis Rheumatol 2017; 69(9):1796-1806). Classification criteria for AS were proposed based on clinical grounds in the 1960s and later modified to include radiological criteria, known as the modified New York criteria for diagnosis of AS (van der Linden et al. Evaluation of Diagnostic Criteria for Ankylosing Spondylitis. A Proposal for Modification of the New York Criteria. Arthritis Rheum. 1984; 27(4):361-8). More recently, the Assessment of SpondyloArthritis international Society (ASAS) formulated classification criteria for axial spondyloarthritis (axSpA), of which AS is considered the prototype disease, based on imaging, clinical, and laboratory criteria (Rudwaleit et al. The development of Assessment of SpondyloArthritis international Society classification criteria for axial spondyloarthritis (part I): classification of paper patients by expert opinion including uncertainty appraisal. Ann Rheum Dis. 2009; 68(6):770-6; Rudwaleit et al. The development of Assessment of SpondyloArthritis international Society classification criteria for axial spondyloarthritis (part II): validation and final selection. Ann Rheum Dis 2009; 68(6):777-83). Disease classification of axSpA is established in persons with a history of back pain for 3 or more consecutive months before reaching 45 years of age, the presence of sacroiliitis confirmed on magnetic resonance imaging (MRI) or plain radiography, and with at least one clinical or laboratory finding that is characteristic of spondyloarthritis (SpA). Alternatively, persons with this history who have a positive test result for HLA-B27 and ≥2 clinical or laboratory features of SpA also fulfill the classification criteria for axSpA. Individuals with axSpA who have established radiographic evidence of sacroiliitis are considered to have met the definition for AS (Rudwaleit et al. The development of Assessment of SpondyloArthritis international Society classification criteria for axial spondyloarthritis (part I): classification of paper patients by expert opinion including uncertainty appraisal. Ann Rheum Dis. 2009; 68(6):770-6; Rudwaleit et al. The development of Assessment of SpondyloArthritis international Society classification criteria for axial spondyloarthritis (part II): validation and final selection. Ann Rheum Dis 2009; 68(6):777-83).

The treatment goal in patients with AS is to optimize long-term health-related quality of life and social participation through control of signs and symptoms, prevention of structural damage, normalization or preservation of function, avoidance of toxicities and minimization of comorbidities (Smolen et al. Treating axial spondyloarthritis and peripheral spondyloarthritis, especially psoriatic arthritis, to target: 2017 update of recommendations by an international task force. Am Rheum Dis. 2018; 77(1):3-17). Current treatment guidelines for active AS (Bath Ankylosing Spondylitis Disease Activity Index [BASDAI] of at least 4, or Ankylosing Spondylitis Disease Activity Score—C-reactive protein [ASDAS-CRP] of at least 2.1) strongly recommend the use of nonsteroidal anti-inflammatory drugs (NSAIDs) and conditionally recommend their continuous use, based on very low-quality evidence (van der Heijde D, et al. 2016 update of the ASAS-EULAR management recommendations for axial spondyloarthritis. Ann Rheum Dis. 2017; 76(6):978-91). Tumor necrosis factor (TNF) blockers and anti-IL-17A monoclonal antibody (mAb) agents have become standard of care for patients who are unresponsive or intolerant to NSAIDs. Based on results of pivotal trials of currently approved biologics in AS, about 30% to 40% of patients treated with biologics do not achieve an Assessment of SpondyloArthritis International Society Response Criteria with an improvement of at least 20% (ASAS 20) and up to 64% of patients do not achieve an Assessment of SpondyloArthritis International Society Response Criteria with an improvement of at least 40% (ASAS 40) (Sieper et al. Secukinumab efficacy in anti-TNF-naive and anti-TNF-experienced subjects with active ankylosing spondylitis: results from the MEASURE 2 Study. Ann Rheum Dis. 2017; 76:571-75; Deodhar et al. Efficacy and Safety of Ixekizumab in the Treatment of Radiographic Axial Spondyloarthritis: Sixteen-Week Results From a Phase III Randomized, Double-Blind, Placebo-Controlled Trial in Patients With Prior Inadequate Response to or Intolerance of Tumor Necrosis Factor Inhibitors. Arthritis Rheumatol 2019; 71(4):599-611).

Although biologics can reduce inflammation and improve symptoms, there is only indirect evidence that currently available biologic TNF blockers influence spinal radiographic progression (Haroon et al. Effect of TNF-alpha inhibitor treatment on bone mineral density in patients with ankylosing spondylitis: A systematic review and meta-analysis. Semin Arthritis Rheum. 2014; 44(22):155-61; Maas et al. Reduction in Spinal Radiographic Progression in Ankylosing Spondylitis Patients Receiving Prolonged Treatment With Tumor Necrosis Factor Inhibitors. Arthritis Care Res (Hoboken). 2017; 69(7):1011-19; Molnar et al. TNF blockers inhibit spinal radiographic progression in ankylosing spondylitis by reducing disease activity: results from the Swiss Clinical Quality Management cohort. Ann Rheum Dis. 2018; 77(1):63-69), which continues to occur in spite of treatment (Poddubnyy et al. Physical Function and Spinal Mobility Remain Stable Despite Radiographic Spinal Progression in Patients with Ankylosing Spondylitis Treated with TNF-α Inhibitors for Up to 10 Years. J Rheumatol 2016; 43(12); 2142-8). Biologics require parenteral administration and are associated with development of autoantibodies, which may be neutralizing and limit drug effectiveness. In addition, profound TNF inhibition by currently available TNF-directed biologics is associated with increased risks of serious infections and malignancies.

In some embodiments, the present disclosure provides the recognition that AS patients who fail or cannot tolerate NSAIDs, and those who have also failed therapy with biologic agents, represent a patient population with high unmet medical need for whom there are currently no approved oral medications available to treat the underlying disease.

In some embodiments, the present disclosure provides a method for treating or lessening the severity of ankylosing spondylitis in a patient, comprising administering to the patient a composition comprising a compound provided herein. In some embodiments, a composition comprising a compound provided herein is administered to a subject who has radiologically confirmed AS. In some such embodiments, the subject has had an inadequate response to nonsteroidal anti-inflammatory drugs (NSAIDs).

In some embodiments, the term “treating or lessening the severity of ankylosing spondylitis” refers to the improvement of long-term health-related quality of life and social participation through one or more of (i) control of signs and symptoms of AS, (ii) prevention of structural damage, (iii) normalization or preservation of function, and (iv) avoidance of toxicities and minimization of comorbidities.

In some embodiments, the present disclosure provides a method of administering a composition comprising a compound provided herein to a subject who is HLA-B-27-positive. In some embodiments, provided methods comprise administering a composition comprising a compound provided herein to a subject in need thereof, wherein the subject is suffering from chronic inflammation associated with or mediated by one or more lymphocytes and/or cytokines. In some such embodiments, the one or more lymphocytes and/or cytokines is or are selected from CD4+ T lymphocytes, CD8+ T lymphocytes, innate-like lymphocytes, tumor necrosis factor (TNF)-α, and IL-17A.

In some embodiments, the present disclosure provides a method of administering a composition comprising a compound provided herein to a subject who satisfies the classification criteria for axial spondyloarthritis (axSpA). In some such embodiments, the classification criteria for axSpA is based on imaging, clinical, and laboratory criteria. In some embodiments, a subject has or is diagnosed with radiographic axSpA. In some embodiments, a subject has or is diagnosed with non-radiographic axSpA. Such subjects exhibit clinical signs and symptoms of SpA but does not exhibit characteristic radiographic changes on pelvic X-rays.

In some embodiments, a subject who satisfies the classification criteria for axSpA is a subject who has a history of back pain for 3 or more consecutive months before reaching 45 years of age, confirmed sacroiliitis, and at least one clinical or laboratory finding that is characteristic of spondyloarthritis (SpA). As used herein, “confirmed sacroiliitis” means sacroiliitis that is or has been confirmed on magnetic resonance imaging (MRI) or plain radiography. In some embodiments, a subject who satisfies the classification criteria for axSpA is a subject who has a positive test result for HLA-B27 and >2 clinical or laboratory features of SpA. In some embodiments, a subject suffering from AS is a subject who has axSpa and has established radiographic evidence of sacroiliitis.

In some embodiments, the present disclosure provides a method of preventing or slowing the progression of structural damage and/or preservation of function in a subject who is suffering from or has been diagnosed with ankylosing spondylitis. In some embodiments, a subject suffering from or diagnosed with ankylosing spondylitis exhibits one or more of the following criteria:
a. low back pain and stiffness for more than 3 months that improves with exercise, but is not relieved by rest;
b. limitation of motion of the lumbar spine in the sagittal and frontal planes;
c. limitation of chest expansion relative to normal values correlated for age and sex; and
d. sacroiliitis grade ≥2 bilaterally or grade 3 to 4 unilaterally.

In some embodiments, a subject has been diagnosed with AS according to the Modified New York Criteria for Ankylosing Spondylitis (1984). In some embodiments, a subject has symptoms of active AS based on a Bath Ankylosing Spondylitis Disease Activity Index (BASDAI) score ≥4. In some embodiments, a subject has a total Back Pain Numerical Rating Scales (NRS) score ≥4. In some embodiments, a subject meets one or more of the following criteria:
a. diagnosed with AS according to the Modified New York Criteria for Ankylosing Spondylitis (1984);
b. symptoms of active AS based on a BASDAI score ≥4; and
c. a total Back Pain Numerical Rating Scales (NRS) score ≥4.

In some embodiments, the present disclosure provides a method of treating AS in a subject, the method comprising:
administering to the subject a composition comprising a compound provided herein, wherein the subject experiences improvement or response in at least three of the following Assessment in SpondyloArthritis International Society (ASAS) criteria:
   a. patient global assessment of disease;
   b. total back pain;
   c. function; and
   d. inflammation.

In some embodiments, the subject experiences improvement or response in at least three of the ASAS criteria of at least 20% and a minimum of one unit on a scale of 0 to 10 and, for the remaining criterion, the subject experiences no worsening from baseline of no more than 20% and a minimum of one unit on a scale of 0 to 10. In some such embodiments, such improvement or response criteria are known as the “ASAS 20 improvement criteria.”

Accordingly, in some embodiments, the present disclosure provides a method of treating AS in a subject, the method comprising:
administering to the subject a composition comprising a compound provided herein, wherein the subject experiences improvement or response of at least 20% and a minimum of one unit in at least three of the following ASAS criteria:
   a. patient global assessment of disease (0 to 10 numerical rating scale);
   b. total back pain (0 to 10 numerical rating scale);
   c. function (assessed by Bath Ankylosing Spondylitis Functional Index (BASFI)); and
   d. inflammation (mean of numerical rating scales for Questions #5 and #6 on Bath Ankylosing Spondylitis Disease Activity Index (BASDAI)); and
wherein, for the remaining criterion, the subject experiences no worsening from baseline of greater than 20% and a minimum of one unit on a scale of 0 to 10.

In some embodiments, the present disclosure provides a method of improving disease activity (e.g., signs and symptoms of AS) in a subject who is suffering from or has been diagnosed with AS, the method comprising administering to the subject a composition comprising a compound provided herein, wherein disease activity is assessed by the ASAS 20 improvement criteria.

In some embodiments, the subject experiences improvement or response in at least three of the ASAS criteria of at least 40% and a minimum of two units on a scale of 0 to 10 and, for the remaining criterion, the subject experiences no worsening from baseline. In some embodiments, the subject experiences improvement or response in at least three of the ASAS criteria of at least 40% and a minimum of two units on a scale of 0 to 10 and, for the remaining criterion, the subject experiences no worsening from baseline of no more than 20% and a minimum of one unit on a scale of 0 to 10. In some such embodiments, such improvement or response criteria are known as the “ASAS 40 improvement criteria.”

Accordingly, in some embodiments, the present disclosure provides a method of treating AS in a subject, the method comprising:
administering to the subject a composition comprising a compound provided herein, wherein the subject experiences improvement or response of at least 40% and a minimum of two units in at least three of the following ASAS criteria:
   a. patient global assessment of disease (0 to 10 numerical rating scale);
   b. total back pain (0 to 10 numerical rating scale);
   c. function (assessed by Bath Ankylosing Spondylitis Functional Index (BASFI)); and
   d. inflammation (mean of numerical rating scales for Questions #5 and #6 on Bath Ankylosing Spondylitis Disease Activity Index (BASDAI)); and
wherein, for the remaining criterion, the subject experiences no worsening from baseline of greater than 20% and a minimum of one unit on a scale of 0 to 10.

In some embodiments, the present disclosure provides a method of improving disease activity (e.g., signs and symptoms of AS) in a subject who is suffering from or has been diagnosed with AS, the method comprising administering to the subject a composition comprising a compound provided herein, wherein disease activity is assessed by the ASAS 40 improvement criteria.

In some embodiments, the present disclosure provides a method of improving disease activity (e.g., signs and symptoms of AS) in a subject who is suffering from or has been diagnosed with AS, the method comprising administering to the subject a composition comprising a compound provided herein, wherein disease activity is assessed by the Ankylosing Spondylitis Disease Activity Score—C-reactive protein (ASDAS-CRP). In some embodiments, the subject achieves a ASDAS-CRP score of ≥1.1. In some such embodiments, the subject achieves a ASDAS-CRP score of ≥2.0. In some such embodiments, the subject achieves a ASDAS-CRP score of <1.3.

In some embodiments, the present disclosure provides a method of improving disease activity (e.g., signs and symptoms of AS) in a subject who is suffering from or has been diagnosed with AS, the method comprising administering to the subject a composition comprising a compound provided herein, wherein disease activity is assessed by the Bath Ankylosing Spondylitis Disease Activity Index (BASDAI).

In some embodiments, the present disclosure provides a method of improving physical function in a subject who is suffering from or has been diagnosed with AS, the method comprising administering to the subject a composition comprising a compound provided herein, wherein physical function is assessed by the Bath Ankylosing Spondylitis Functional Index (BASFI).

In some embodiments, the present disclosure provides a method of reducing spinal and sacroiliac joint inflammation in a subject who is suffering from or has been diagnosed with AS, the method comprising administering to the subject a composition comprising a compound provided herein, wherein spinal and sacroiliac joint inflammation is assessed by Spondylarthritis Research Consortium of Canada (SPARCC) MRI score of sacroiliac joints and spine.

In some embodiments, a subject suffering from or diagnosed with ankylosing spondylitis has failed therapy with at least 2 nonsteroidal anti-inflammatory drugs (NSAIDs). In some embodiments, a subject suffering from or diagnosed with ankylosing spondylitis has not received therapy selected from one or more of:
a. a cell depleting biologic agent such as an anti-CD20 antibody (e.g., rituximab), an anti-CD4 antibody, an anti-CD3 antibody, denosumab, an anti-IL-6 antibody (e.g., tocilizumab and sarilumab), and an anti-IL-23 antibody (e.g., ustekinuma) for at least 6 months prior to administration of a compound provided herein;
b. an oral corticosteroid (e.g., prednisone, etc.) in an amount greater than 10 mg/day systemically for at least 2 weeks prior to administration of a compound provided herein;
c. an intramuscular, intravenous, or intraarticular corticosteroid in any amount within at least 4 weeks of administration of a compound provided herein;
d. a vitamin K antagonist (e.g., warfarin);
e. isoniazid within at least 4 weeks of administration of a compound provided herein; and
f. any medication that is a substrate of one or more of the following transporters and has a narrow therapeutic index: p-glycoprotein (P-gp) (e.g., aliskiren, ambrisentan, colchicine, cyclosporine, dabigatran etexilate, digoxin, everolimus, fexofenadine, methotrexate, ranolazine, rivaroxaban, saxagliptin, sirolimus, sitagliptin, talinolol, ticagrelor, tolvaptan, etc.), breast cancer resistance protein (BCRP) (e.g., methotrexate, sulfasalazine, leflunomide, rosuvastatin, etc.), organic cation transporter 1 (OCT1) (e.g., metformin, gabapentin, pramipexole, tramadol, varenicline, etc.), organic anion transporting polypeptides 1B1 and 1B3 (OATP1B1 and OATP1B3, respectively) (e.g., ambrisentan, atorvastatin, ezetimibe, fluvastatin, glyburide, rosuvastatin, simvastatin acid, pitavastatin, pravastatin, repaglinide, telmisartan, valsartan, olmesartan, mycophenolic acid, etc.).

In some embodiments, a subject suffering from or diagnosed with ankylosing spondylitis has failed therapy with at least 2 nonsteroidal anti-inflammatory drugs (NSAIDs) and not more than 1 biological agent. In some embodiments, a subject who has failed therapy with not more than 1 biological agent is a subject who has had, for at least 12 weeks, an inadequate response and/or an unacceptable safety/tolerability to at least 1 dose of a biologic agent for AS (e.g., a TNF antagonist or IL-17A monoclonal antibody).

In some such embodiments, the patient is administered a composition comprising a therapeutically effective amount of a compound provided herein. In some embodiments, the patient is administered a unit dose of a compound provided herein.

In some embodiments, the present disclosure provides a use of a compound provided herein in the manufacture of a medicament for treating ankylosing spondylitis. In some embodiments, the present disclosure provides a compound described herein for use in treating ankylosing spondylitis.

Biomarkers of AS. In some embodiments, the present disclosure provides a method of administering to a subject a composition comprising a compound provided herein and monitoring the level of one or more biomarkers associated or correlated with AS. Pro-inflammatory cytokines and chemokines, including TNF-α, monocyte chemoattractant protein-1 (MCP-1), and IL-17A, have been shown to be increased in AS patients (Braun et al. Anti-tumour necrosis factor a therapy for ankylosing spondylitis: international experience. Ann Rheum Dis 2002; 61(Suppl III):iii51-iii60; West et al. Oncostatin M drives intestinal inflammation and predicts response to tumor necrosis factor-neutralizing therapy in patients with inflammatory bowel disease. Nat Med. 2017; 23(5)579-89; Romero-Sanchez et al. Serum monocyte chemotactic protein-1 concentrations distinguish patients with ankylosing spondylitis from patients with mechanical low back pain. J Spinal Disord Tech. 2011; 24(3):202-7). Additionally, increased serum levels of TNF-α have been correlated with increased CRP levels in AS patients (Wagner et al. Serum markers associated with clinical improvement in patients with ankylosing spondylitis treated with golimumab. Am Rheum Dis. 2012; 71(5):674-80). Accordingly, in some embodiments, a biomarker associated or correlated with AS is selected from a pro-inflammatory cytokine or chemokine. In some such embodiments, a pro-inflammatory cytokine or chemokine is selected from TNF-α, monocyte chemoattractant protein-1 (MCP-1), and IL-17A. In some embodiments, the level of a pro-inflammatory cytokine or chemokine decreases over a period of time relative to a reference standard. In some such embodiments, a reference standard is the level of the pro-inflammatory cytokine or chemokine for a given subject or a given population prior to exposure to a compound provided herein.

In AS, several bone remodeling processes take place simultaneously: pathologic new bone formation in the form of syndesmophytes and bone loss in the form of bone erosion, osteolysis, and bone mineral density (BMD) loss leading to osteoporosis (Klingberg et al. Osteoporosis in ankylosing spondylitis—prevalence, risk factors and methods of assessment. Arthritis Res Ther 2012:14(3):R108). In some embodiments, a biomarker associated or correlated with AS is a bone formation marker. In some such embodiments, a bone formation marker is selected from procollagen type 1 N-terminal propeptide (P1NP) and bone resorption markers such as carboxy terminal cross-linked telopeptide of type 1 collagen (CTX-1). In some embodiments, the level of a bone formation marker decreases over a period of time relative to a reference standard. In some embodiments, the level of a bone formation marker increases over a period of time relative to a reference standard. In some embodiments, a reference standard is the level of the bone formation marker for a given subject or a given population prior to exposure to a compound provided herein.

Bone destruction is mediated by the recruitment of osteoclast precursors (OCPs) into the inflamed tissue and their differentiation into mature osteoclasts. TNF inhibition has resulted in sustained loss of circulating OCPs that can differentiate into osteoclasts (Lam et al. TNF-alpha induces osteoclastogenesis by direct stimulation of macrophages exposed to permissive levels of RANK ligand. J Clin Invest. 2000; 106(12):1481-8; Li et al. Systemic tumor necrosis factor alpha mediates an increase in peripheral CD11bhigh osteoclast precursors in tumor necrosis factor alpha-transgenic mice. Arthritis Rheum. 2004; 50(1):265-76). In some embodiments, a biomarker associated or correlated with AS is an osteoclast precursor (OCP). In some embodiments, the level of an osteoclast precursor decreases over a period of time relative to a reference standard. In some such embodiments, a reference standard is the level of the osteoclast precursor for a given subject or a given population prior to exposure to a compound provided herein.

In some embodiments, a biomarker associated or correlated with AS is a genetic marker. In some such embodiments, a genetic marker is selected from HLA-B27 and polygenic risk scores built using public AS data (see, e.g., Rostami et al. Prediction of Ankylosing Spondylitis in the HUNT Study by a Genetic Risk Score Combining 110 Single-nucleotide Polymorphisms of Genome-wide Significance. J Rheumatol 2019; 46:1-7).

### Rheumatoid Arthritis

Rheumatoid arthritis is a chronic autoimmune disorder in which the body's immune system attacks its own tissue, including joint linings, synovial tissues, cartilage and bone, causing painful swelling. The inflammation that results from immune system attacks results in the thickening of the synovium, the tissue that lines the insides of joints, leading to swelling and pain in and around the joints. Over long periods of time, the inflammation associated with rheumatoid arthritis can damage cartilage, the elastic tissue that covers the ends of bones in a joint, as well as the bones themselves. Over time, there is loss of cartilage, and the joint spacing between bones can become smaller. Joints can become loose, unstable, painful and lose their mobility. Joint deformity also can occur. Joint damage cannot be reversed, and because it can occur early, doctors recommend early diagnosis and aggressive treatment to control rheumatoid arthritis. In severe cases, rheumatoid arthritis attacks internal organs.

Patients with rheumatoid arthritis can be classified into distinct subsets, including lymphoid, myeloid and fibroid subsets. Dennis et al., “Synovial phenotypes in rheumatoid arthritis correlate with response to biologic therapeutics,” Arthritis Research & Therapy 2014, 16:R90, 1-18; Setiadi, et. al, “Synovial Subset-Derived Baseline Serum Biomarkers Segregate Rheumatoid Arthritis Patients into Subgroups with Distinct Serum Protein and Clinical Characteristics,” Abstract Number 1307, 2013 ACR/ARHP Annual Meeting.

In some embodiments, the present disclosure provides a method of treating rheumatoid arthritis in a patient, comprising administering to the patient a composition comprising a compound provided herein. In some such embodiments, the patient is administered a composition comprising a therapeutically effective amount of a compound provided herein. In some embodiments, the patient is administered a unit dose of a compound provided herein.

In some embodiments, the present disclosure provides a method of treating one or more of the lymphoid, myeloid and fibroid subsets of rheumatoid arthritis, comprising administering a composition comprising a compound provided herein to a patient in one or more subsets. Such subsets are classified by the presence of certain biomarkers which are detailed in Dennis et al., “Synovial phenotypes in rheumatoid arthritis correlate with response to biologic therapeutics,” Arthritis Research & Therapy 2014, 16:R90, 1-18; Setiadi, et. al, “Synovial Subset-Derived Baseline Serum Biomarkers Segregate Rheumatoid Arthritis Patients into Subgroups with Distinct Serum Protein and Clinical Characteristics,” Abstract Number 1307, 2013 ACR/ARHP Annual Meeting, each of which is hereby incorporated by reference.

In some embodiments, the present disclosure provides a method for treating or lessening the severity of rheumatoid arthritis in a patient, wherein the patient has one or more biomarkers for the lymphoid subset of rheumatoid arthritis, comprising administering to the patient a composition comprising a compound provided herein. Such biomarkers for the lymphoid subset of rheumatoid arthritis include, for example, high CXCL13 and low soluble ICAM1 expression levels. In some embodiments, the present disclosure provides a method for treating or lessening the severity of rheumatoid arthritis in a patient, wherein the patient has one or more biomarkers for the myeloid subset of rheumatoid arthritis, comprising administering to the patient a composition comprising a compound provided herein. In some embodiments, the present disclosure provides a method for treating or lessening the severity of rheumatoid arthritis in a patient, wherein the patient has one or more biomarkers for the fibroid subset of rheumatoid arthritis, comprising administering to the patient a composition comprising a compound provided herein. In some embodiments, the present disclosure provides a method for treating or lessening the severity of at least one subset of rheumatoid arthritis, comprising administering to the patient a composition comprising a compound provided herein. In some embodiments, the subset of rheumatoid arthritis is lymphoid. In some embodiments, the subset of rheumatoid arthritis is myeloid. In some embodiments, the subset of rheumatoid arthritis is fibroid.

In some embodiments, the present disclosure provides a use of a compound provided herein in the manufacture of a medicament for treating rheumatoid arthritis. In some embodiments, the present disclosure provides a compound described herein for use in treating rheumatoid arthritis.

### Psoriasis and Psoriatic Arthritis

Psoriasis is a chronic, inflammatory disease of the skin, scalp, nails, and joints that is characterized by a scaly rash that occurs most frequently on the elbows, knees, and scalp, but can cover much of the body. A normal skin cell matures and falls off the body's surface in 28 to 30 days, but a psoriatic skin cell takes only three to four days to mature and gathers at the surface, thus forming lesions.

Up to 30 percent of people with psoriasis also develop psoriatic arthritis. In most cases (though not always), the psoriasis will precede the arthritis, sometimes by many years. When arthritis symptoms occur with psoriasis, it is called psoriatic arthritis (PsA). In these cases, the joints at the end of the fingers are most commonly affected, causing inflammation and pain, but other joints like the wrists, knees, and ankles can also become involved. Symptoms in the fingernails and toenails range from small pits in the nails to nearly complete destruction and crumbling as seen in reactive arthritis or fungal infections.

About 20 percent of patients with PsA will develop spinal involvement, which is called psoriatic spondylitis. Inflammation of the spine can lead to complete fusion, as in ankylosing spondylitis (AS), or affect only certain areas such as the lower back or neck. Patients who are HLA-B27 positive are more likely than others to have their disease progress to the spine.

PsA and AS are considered genetically and clinically related because both are inflammatory rheumatic diseases linked to the HLA-B27 gene. HLA-B27 is a powerful predisposing gene associated with several rheumatic diseases. The gene itself does not cause disease, but can make people more susceptible. While a number of genes are linked to PsA, the highest predictive value is noted with HLA-B27.

In some embodiments, the present disclosure provides a method for treating or lessening the severity of psoriasis and/or psoriatic arthritis in a patient, comprising administering to the patient a composition comprising a compound provided herein. In some such embodiments, the patient is administered a composition comprising a therapeutically effective amount of a compound provided herein. In some embodiments, the patient is administered a unit dose of a compound provided herein.

In some embodiments, the present disclosure provides a use of a compound provided herein in the manufacture of a medicament for treating psoriasis and/or psoriatic arthritis. In some embodiments, the present disclosure provides a compound described herein for use in treating psoriasis and/or psoriatic arthritis.

### EXAMPLES

### General Information

### LCMS Method 1.

Luna C18 (2) 50×3.0 mm, 3.0 um. Temperature: 45° C., Flow: 1.5 mL/min, run time: 2.5 min. Mobile phase conditions: Initial 95% H₂O+0.1% FA/5% MeCN+0.1% FA, then linear gradient to 95% MeCN+0.1% FA over 1.3 min. then hold for 1.2 min. at 95% MeCN+0.1% FA. MSD: ESI Positive

### LCMS Method 2.

SunFire C18 75×4.6 mm, 3.5 um. Temperature: 45° C., Flow: 1.5 mL/min, run time: 6.0 min. Mobile phase conditions: Initial 95% H₂O+0.1% FA/5% MeCN+0.1% FA, then linear gradient to 95% MeCN+0.1% FA over 4.0 min. then hold for 2.0 min. at 95% MeCN+0.1% FA. MSD: ESI Positive

### LCMS Method 3.

Column: HALO C18, 3.0×30 mm, 2.7 pm particles; Mobile Phase A: water with 0.05% trifluoroacetic acid; Mobile Phase B: acetonitrile with 0.05% trifluoroacetic acid; Temperature: 40° C.; Gradient: 5% B to 100% B over 1.3 min, then a 0.50 min hold at 100% B; Flow: 1.5 mL/min

### Example 1. Synthesis of Exemplary Compounds

### Methods of Preparation

Compounds of present disclosure and intermediates used in the preparation of compounds of Formula I, can be prepared using procedures shown in the following examples and related procedures. The methods and conditions used in these examples, and the actual compounds prepared in these examples, are not meant to be limiting, but are meant to demonstrate how the compounds of Formula I can be prepared. Starting materials and reagents used in these examples, when not prepared by a procedure described herein, are generally either commercially available, or are reported in the chemical literature, or may be prepared by using procedures described in the chemical literature.

Abbreviations as used herein, are defined as follows: “1×” for once, “2×” for twice, “3×” for thrice, “° C.” for degrees Celsius, “equiv” for equivalent or equivalents, “g” for gram or grams, “mg” for milligram or milligrams, “L” for liter or liters, “mL” for milliliter or milliliters, “μL” for microliter or microliters, “N” for normal, “M” for molar, “mmol” for millimole or millimoles, “min” for minute or minutes, “h” for hour or hours, “rt” for room temperature, “ON” for overnight, “RT” for retention time, “atm” for atmosphere, “psi” for pounds per square inch, “conc.” for concentrate, “sat” or “saturated” for saturated, “CVs” for column volumes, “MW” for molecular weight, “mp” for melting point, “ee” for enantiomeric excess, “MS” or “Mass Spec” for mass spectrometry, “ESI” for electrospray ionization mass spectroscopy, “HR” for high resolution, “HRMS” for high resolution mass spectrometry, “LCMS” or “LC/MS” for liquid chromatography mass spectrometry, “HPLC” for high pressure liquid chromatography, “RP HPLC” for reverse phase HPLC, “TLC” or “tlc” for thin layer chromatography, “NMR” for nuclear magnetic resonance spectroscopy, “nOe” for nuclear Overhauser effect spectroscopy, “¹H” for proton, “δ” for delta, “s” for singlet, “d” for doublet, “t” for triplet, “q” for quartet, “m” for multiplet, “br” for broad, “MHz” for megahertz, and “α”, “β”, “R”, “S”, “E”, and “Z” are stereochemical designations familiar to one skilled in the art.
AcOH or HOAc acetic acid
B(OMe)₃ trimethyl borate
Boc tert-butyloxycarbonyl
BTEAC benzyltriethylammonium chloride
Bu Butyl
CH₂Cl₂ dichloromethane
CH₃I methyl iodide
Cphos 2-dicyclohexylphosphino-2′,6′-bis(N,N-dimethylamino)biphenyl
Cs₂CO₃ cesium carbonate
CV Column volume
DCM dichloromethane
DIEA/DIPEA/Hünig's Base diisopropylethylamine
DMF dimethyl formamide
DMSO dimethyl sulfoxide
Et Ethyl
EtOAc ethyl acetate
EtOH ethanol
FA formic acid
H₂ hydrogen (gas)
H₂O Water
H₂SO₄ sulfuric acid
HATU O-(7-azabenzotriazol-1-yl)-N,N,N′,N′-tetramethyluronium hexafluorophosphate
HCl hydrochloric acid
i-Pr isopropyl
K₂CO₃ potassium carbonate
K₃PO₄ potassium phosphate
LiOH lithium hydroxide
Me Methyl
MeCN acetonitrile
MeOH Methanol
MTBE Methyl tert-butyl ether
N₂ nitrogen (gas)
Na₂SO₄ sodium sulfate
NaBH(OAc)₃ sodium triacetoxyborohydride
NaBH₃CN sodium cyanoborohydride
NaHCO₃ sodium bicarbonate
n-BuLi n-butyllithium
NH₄Cl ammonium chloride
Pd(OH)₂/C palladium hydroxide on carbon
Pd(PPh₃)₄ palladium tetrakis
Pd/C palladium on carbon
Pd₂(dba)₃ tris(dibenzylideneacetone)dipalladium(0)
PdCl₂(dppf) [1,1′-bis(diphenylphosphino)-ferrocene]dichloropalladium(II)
PE petroleum ether
PF₆ hexafluorophosphate
pin Pinacolato
Ruphos 2-dicyclohexylphosphino-2′,6′-diisopropoxybiphenyl
Ruphos Pd G3 (2-dicyclohexylphosphino-2′,6′-diisopropoxy-1,1′-biphenyl)[2-(2′-amino-1,1′-biphenyl)]palladium(II) methanesulfonate
SiO₂ silica oxide
t-BuOK Potassium tert-butoxide
TEA triethylamine
TFA trifluoroacetic acid
TfOH triflic acid
THF tetrahydrofuran
Xphos 2-dicyclohexylphosphino-2′,4′,6′-triisopropylbiphenyl
Xphos Pd G3 2-dicyclohexylphosphino-2′,4′,6′-triisopropyl-1,1′-biphenyl)[2-(2′-amino-1,1′-biphenyl)]palladium(II) methanesulfonate
ZnCl₂ zinc chloride

The compounds of the present disclosure may be synthesized by many methods available to those skilled in the art of organic chemistry (Smith, M. B., March's Advanced Organic Chemistry: Reactions, Mechanisms, and Structure, 7^{th} Edition (2013)). General synthetic schemes for preparing compounds of the present invention are described below. These schemes are illustrative and are not meant to limit the possible techniques one skilled in the art may use to prepare the compounds disclosed herein. Different methods to prepare the compounds of the present invention will be evident to those skilled in the art. Additionally, the various steps in the synthesis may be performed in an alternate sequence in order to give the desired compound or compounds.

Examples of compounds of the present disclosure prepared by methods described in the general schemes are given in the intermediates and examples section set out hereinafter. Example compounds are typically prepared as racemic mixtures. Preparation of homochiral examples may be carried out by techniques known to one skilled in the art. For example, homochiral compounds may be prepared by separation of racemic products by chiral phase preparative HPLC. Alternatively, the example compounds may be prepared by methods known to give enantiomerically enriched products. These include, but are not limited to, the incorporation of chiral auxiliary functionalities into racemic intermediates which serve to control the diastereoselectivity of transformations, providing enantio-enriched products upon cleavage of the chiral auxiliary.

Scheme 1 illustrates an approach to the synthesis of compounds exemplified by 8a. Intermediate 3 can be synthesized through Pd-catalyzed Suzuki cross-coupling (Miyaura, N. and Suzuki, A. Chemical Reviews, 95:2457-2483, 1995) of 1 (previously reported: Anderson et al., J. Med. Chem., 2007, 50, 2647-2654) and 2. The resulting ester 3 can be saponified to the desired acid 4 through treatment with a base, such as LiOH.

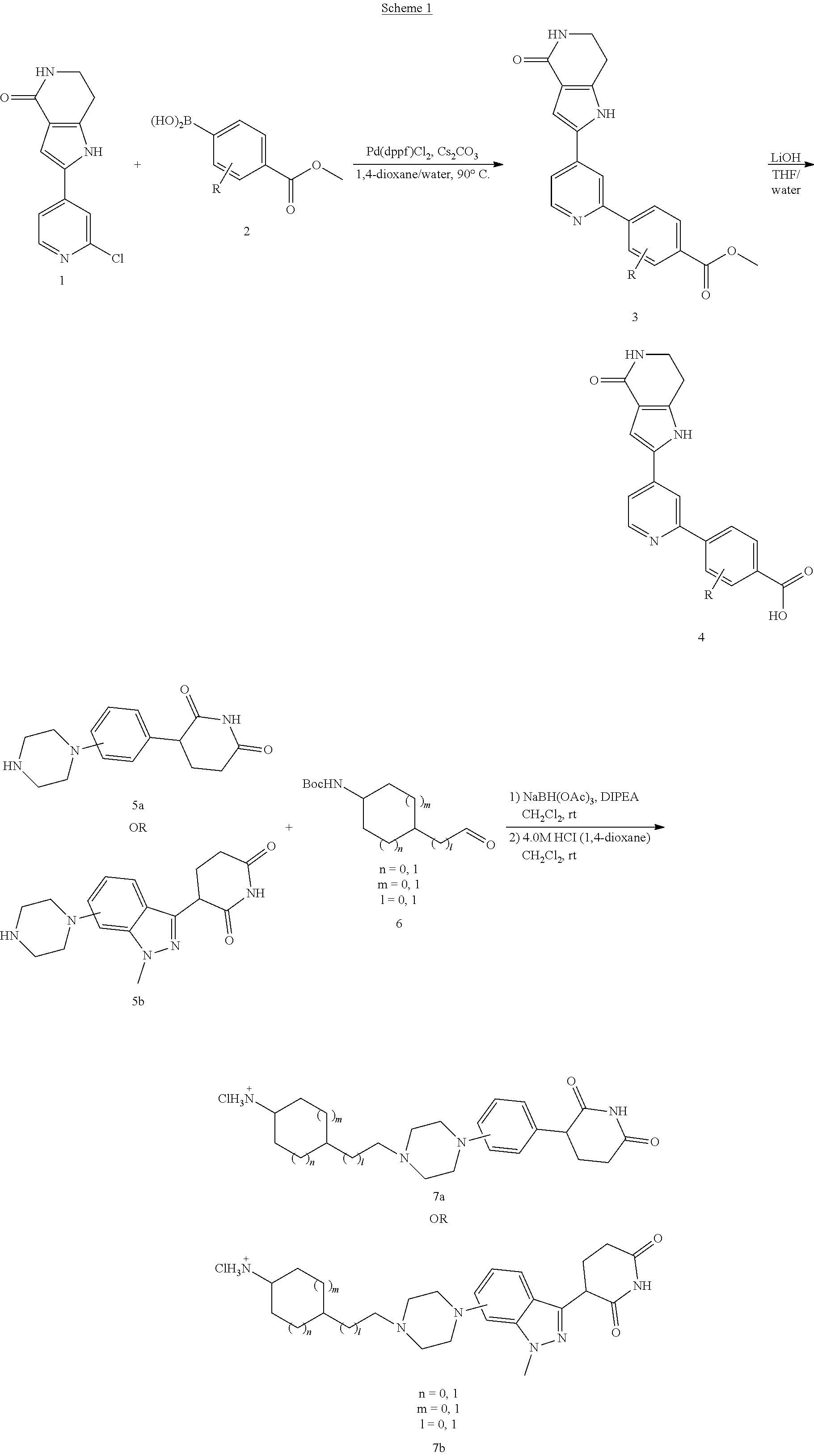

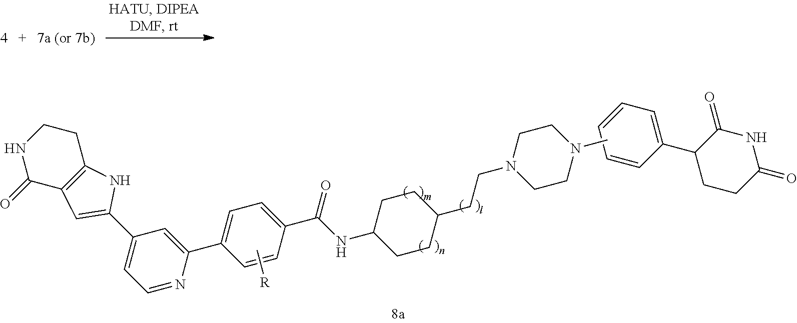

Synthesis of amine coupling partners 7a and 7b can be carried out through reductive amination (Afanasyev, O. I. et al. Chemical Reviews, 119:11857-11911, 2019) of 6a or 6b with aldehyde 6 by treatment with NaBH(OAc)₃ in the presence of a base. Deprotection of the resulting boc-protected amines can be carried out by treatment with acid, such as 4.0 M HCl in 1,4-dioxane generating 7a or 7b. Lastly, coupling of 4 with 7a (or 7b) through amide formation by a coupling reagent, such as HATU, in the presence of base. Alternative amidation conditions known to those in the art may also be used in this step.

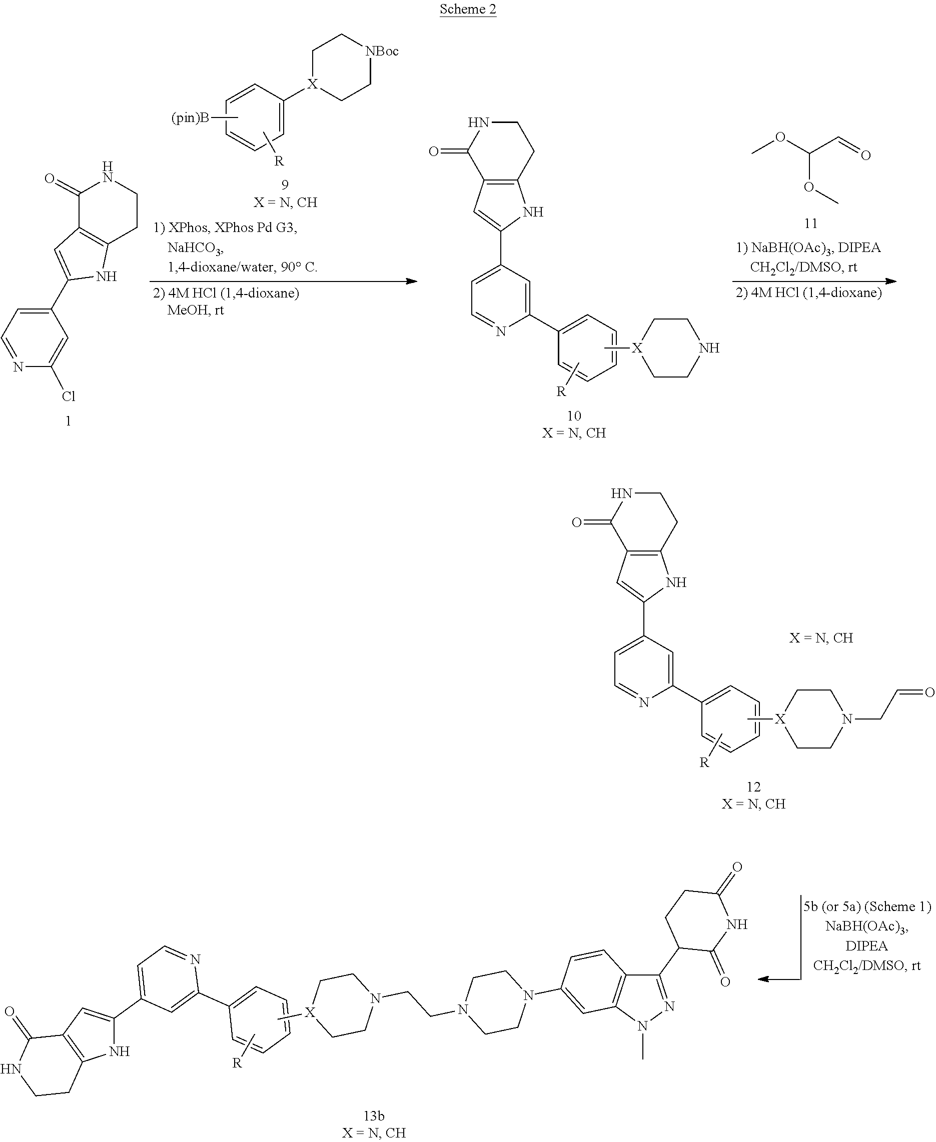

Scheme 2 illustrates an approach to the synthesis of compounds exemplified by 13b. 1 can be converted to secondary amine 10 by a two-step procedure of Suzuki cross-coupling with a boronate ester followed by acid-promoted boc-deprotection of the amine. Boronic acid, potassium trifluoroborate salt, or MIDA-boronate intermediates would also be functional under similar conditions. Aldehyde 12 can be synthesized by reductive amination with aldehyde 11, followed by acid-promoted deprotection of the resulting methoxy acetal. Alkylation with an analogous halogen-substituted acetal could also be used. Reductive amination of 12 with 7a or 7b would generate compounds exemplified by 13b.

Purification of intermediates and final products was carried out via either normal or reverse phase chromatography. Normal phase chromatography on an ISCO system was carried out using prepacked SiO₂ cartridges eluting with either gradients of hexanes and EtOAc or DCM and MeOH unless otherwise indicated. Reverse phase preparative HPLC or LCMS was carried out using C18 columns eluting with gradients of Solvent A (90% water, 10% MeOH, 0.1% TFA) and Solvent B (10% water, 90% MeOH, 0.1% TFA, UV 220 nm), or with gradients of Solvent A (95% water, 5% MeCN, 0.1% TFA) and Solvent B (5% water, 95% MeCN, 0.1% TFA, UV 220 nm), or with gradients of Solvent A (98% water, 2% MeCN, 0.05% TFA) and Solvent B (98% MeCN, 2% water, 0.05% TFA, UV 254 nm), or with gradients of Solvent A (95% water, 5% MeCN with 10 mM ammonium acetate) and Solvent B (95% MeCN, 5% water with 10 mM ammonium acetate).

In most examples, one of the following LC/MS conditions were utilized to determine purity:

### LC/MS Method 1

Luna C18 (2) 50×3.0 mm, 3.0 mm. Temperature: 45° C., Flow: 1.5 mL/min, run time: 2.5 min. Mobile phase conditions: Initial 95% H₂O+0.1% FA/5% CH₃CN+0.1% FA, then linear gradient to 95% CH₃CN+0.1% FA over 1.3 min. then hold for 1.2 min. at 95% CH₃CN+0.1% FA. MSD: ESI Positive

### LC/MS Method 2

SunFire C18 75×4.6 mm, 3.5 pm. Temperature: 45° C., Flow: 1.5 mL/min, run time: 6.0 min. Mobile phase conditions: Initial 95% H₂O+0.1% FA/5% CH₃CN+0.1% FA, then linear gradient to 95% CH₃CN+0.1% FA over 4.0 min. then hold for 2.0 min. at 95% CH₃CN+0.1% FA. MSD: ESI Positive

### LC/MS Method 3

Column: HALO C18, 3.0×30 mm, 2.7 pm particles; Mobile Phase A: H₂O with 0.05% trifluoroacetic acid; Mobile Phase B: CH₃CN with 0.05% trifluoroacetic acid; Temperature: 40° C.; Gradient: 5% B to 100% B over 1.3 min, then a 0.50 min hold at 100% B; Flow: 1.5 mL/min

### Example 1.1. Synthesis of Intermediate 3-(4-(Piperazin-1-yl)phenyl)piperidine-2,6-dione

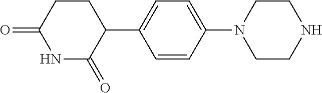 [SMILES: O=C1CCC(c2ccc(N3CCNCC3)cc2)C(=O)N1]

(2,6-Bis(benzyloxy)pyridin-3-yl)boronic acid. Four batches carried out: to a solution of 2,6-bis(benzyloxy)-3-bromopyridine (400 g, 1.08 mol, 1.0 equiv) in THF (4 L) was added drop wise n-BuLi (2.5 M, 475 mL, 1.1 equiv) at −70° C., then the solution was stirred at −70° C. for 0.5 h. After that, B(OMe)₃ (146 g, 1.40 mol, 159 mL, 1.3 equiv) was added drop wise to the reaction solution at −70° C. and the solution was further stirred at −70° C. for 0.5 h. The batches were combined and the reaction solution was poured into sat. aq. NH₄Cl (15 L). The organic layer was separated and the aqueous phase was extracted with EtOAc (5 L, then 3 L). The combined organic layers were washed with brine (5 L), dried over Na₂SO₄, filtered and concentrated to give a residue. The crude was triturated with (PE/EtOAc=10/1, 1.5 L) for 1 h. The solid was collected by filtration and dried under vacuum at 45° C. for 2 h to give 3-(4-(piperazin-1-yl)phenyl)piperidine-2,6-dione (980 g, 2.77 mol, 64% yield, 95% purity) as a light blue solid. ¹H NMR (400 MHz, CDCl₃) δ 8.05 (d, J=8.0 Hz, 1H), 7.27-7.44 (m, 10H), 6.48 (d, J=8.0 Hz, 1H), 5.93 (s, 2H), 5.45 (s, 2H), 5.38 (s, 2H).

tert-Butyl 4-(4-(2,6-bis(benzyloxy)pyridin-3-yl)phenyl)piperazine-1-carboxylate. Two batches carried out: a mixture of tert-butyl 4-(4-bromophenyl)piperazine-1-carboxylate (220 g, 645 mmol, 1.0 equiv), (2,6-Bis(benzyloxy)pyridin-3-yl)boronic acid (261 g, 677 mmol, 87% purity, 1.05 equiv), K₃PO₄ (2 M, 645 mL, 1.29 mol 2.0 equiv), Pd(PPh₃)₄ (37.3 g, 32.2 mmol, 0.05 equiv) in 1,4-dioxane (2.2 L) was degassed and purged with N₂ 3 times. The mixture was then stirred at 90° C. for 16 h under N₂ atmosphere. The batches were combined and the mixture was cooled to 20° C. and filtered. The filtrate was extracted with EtOAc (3×500 mL). The combined organic phase was washed with brine (500 mL), dried with anhydrous Na₂SO₄, filtered and concentrated under vacuum to give a residue. The residue was purified by column chromatography (SiO₂, PE/EtOAc=50/1 to 0/1). tert-Butyl 4-(4-(2,6-bis(benzyloxy)pyridin-3-yl)phenyl)piperazine-1-carboxylate (670 g, crude) was obtained as a green solid. ¹H NMR (400 MHz, CDCl₃) δ 7.59-7.61 (m, 1H), 7.50-7.53 (m, 2H), 7.34-7.43 (m, 10H), 6.96 (d, J=8.8 Hz, 2H), 6.70 (d, J=8.0 Hz, 1H), 5.44 (s, 2H), 5.37 (s, 2H), 3.61 (t, J=5.2 Hz, 4H), 3.18 (t, J=5.2 Hz, 4H), 1.51 (s, 9H).

tert-Butyl 4-(4-(2,6-dioxopiperidin-3-yl)phenyl)piperazine-1-carboxylate. Seven batches carried out: to a mixture of tert-butyl 4-(4-(2,6-bis(benzyloxy)pyridin-3-yl)phenyl)piperazine-1-carboxylate (85 g, 154 mmol, 1.0 equiv) in THF (400 mL) and EtOH (400 mL) was added 10% Pd/C (20 g), 20% Pd(OH)₂/C (20 g) and AcOH (9.25 g, 154 mmol, 1.0 equiv) under N₂. The suspension was degassed under vacuum and purged with H₂ several times. The mixture was stirred at 50° C. for 12 h under H₂ (40 psi). The seven batches were combined and the reaction mixture was filtered and the filtrate was concentrated. The crude product from the filtrate was triturated with EtOAc (500 mL) at 15° C. for 30 min to give tert-butyl 4-(4-(2,6-dioxopiperidin-3-yl)phenyl)piperazine-1-carboxylate 220 g (100% purity) and 180 g of a second batch containing impurities. The 180 g crude material was purified by column chromatography (SiO₂, PE/EtOAc=20/1 to 0/1) to give 100 g product, tert-Butyl 4-(4-(2,6-dioxopiperidin-3-yl)phenyl)piperazine-1-carboxylate (320 g, 857 mmol, 80% combined yield) was obtained as a white solid. ¹H NMR (400 MHz, CDCl₃) δ ppm 10.8 (s, 1H), 7.07 (d, J=8.8 Hz, 2H), 6.92 (d, J=8.8 Hz, 2H), 3.74 (dd, J=11.2 Hz, 6.4 Hz, 1H), 3.44-3.45 (m, 4H), 3.07 (t, J=6.4 Hz, 4H), 2.61-2.64 (m, 1H), 2.44-2.48 (m, 1H), 2.13 (m, 1H), 2.01-2.02 (m, 1H), 1.42 (s, 9H).

3-(4-(Piperazin-1-yl)phenyl)piperidine-2,6-dione. Two batches carried out: To a solution of tert-butyl 4-(4-(2,6-dioxopiperidin-3-yl)phenyl)piperazine-1-carboxylate (105 g, 281 mmol, 1.0 equiv) in DCM (600 mL) cooled to 15° C. was added dropwise HCl/EtOAc (4 M, 500 mL). After addition, the mixture was stirred at 15° C. for 3 h. The two batches were combined and the reaction mixture was filtered and the filter cake was air dried. The crude product was triturated with EtOAc (500 mL) at 15° C. for 30 min. 3-(4-(piperazin-1-yl)phenyl)piperidine-2,6-dione, 2 HCl (194 g, 560 mmol, quantitative yield) was obtained as a white solid. ¹H NMR (400 MHz, DMSO-d6) δ 10.8 (s, 1H), 9.24 (s, 2H), 7.11 (d, J=8.4 Hz, 2H), 6.96 (d, J=8.8 Hz, 2H), 3.77 (q, J=5.2 Hz, 1H), 3.34-3.36 (m, 4H), 3.20 (s, 4H), 2.61-2.64 (m, 1H), 2.44-2.48 (m, 1H), 2.14 (m, 1H), 1.90-2.01 (m, 1H).

### Example 1.2. Synthesis of Intermediate 3-(3-Bromophenyl)piperidine-2,6-dione

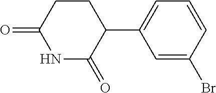 [SMILES: O=C1CCC(c2cccc(Br)c2)C(=O)N1]

tert-Butyl 4-(3-bromophenyl)-4-cyanobutanoate. To a solution 2-(3-bromophenyl) acetonitrile (70 g, 357 mmol) in toluene (700 mL) at 20° C. was added tert-butyl prop-2-enoate (45.7 g, 357 mmol), BTEAC (8.13 g, 35.7 mmol) and K₂CO₃ (49.4 g, 357 mmol). After addition, the reaction mixture was stirred at 75° C. for 4.5 h. HPLC showed 17% conversion to product. The reaction was filtered and to the filtrate was added EtOAc (1 L). The organic layer was washed with water (2×500 mL), brine (2×500 mL), dried over anhydrous Na₂SO₄, filtered and concentrated to afford tert-butyl 4-(3-bromophenyl)-4-cyano-butanoate (115 g, crude) as brown oil, which was used in the next step without further purification.

3-(3-Bromophenyl)piperidine-2,6-dione. To a solution of tert-butyl 4-(3-bromophenyl)-4-cyano-butanoate (131 g, 404 mmol) in AcOH (800 mL) at 20° C. was added H₂SO₄ (7.93 g, 80.8 mmol). After addition, the reaction mixture was stirred at 115° C. for 16 h. HPLC showed 25% conversion to product. The reaction mixture was concentrated, dissolved with EtOAc (2 L), washed with water (2×1 L), brine (2×1 L), dried over anhydrous Na₂SO₄, filtered and concentrated. The residue was washed with MTBE (100 mL). The filter cake was dissolved with EtOAc (1 L) and washed with Sat. aq. NaHCO₃ (3×500 mL), water (2×500 mL), brine (2×500 mL) and dried over anhydrous Na₂SO₄. The mixture was filtered and concentrated to afford 3-(3-bromophenyl) piperidine-2,6-dione (16.6 g, 15% yield) as an off-white solid. ¹H NMR (400 MHz, CDCl₃) δ ppm 8.17 (br. s, 1H), 7.47 (d, J=8.0 Hz, 1H), 7.38 (d, J=2.8, Hz, 1H), 7.28-7.25 (m, 1H), 7.16 (d, J=7.6 Hz, 1H), 3.77-3.73 (m, 1H), 2.78-2.67 (m, 2H), 2.29-2.25 (m, 2H).

### Example 1.3. Synthesis of Intermediate 3-(1-Methyl-6-(piperazin-1-yl)-1H-indazol-3-yl)piperidine-2,6-dione

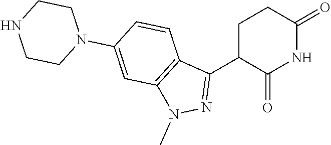 [SMILES: Cn1nc(C2CCC(=O)NC2=O)c2ccc(N3CCNCC3)cc21]

tert-Butyl 4-(3-(2,6-Bis(benzyloxy)pyridin-3-yl)-1-methyl-1H-indazol-6-yl)piperazine-1-carboxylate. A mixture of 6-bromo-3-(2,6-dibenzyloxy-3-pyridyl)-1-methyl-indazole (30.0 g, 60.0 mmol), tert-butyl piperazine-1-carboxylate (16.8 g, 89.9 mmol), RuPhos-Pd-G3 (10 g, 12.0 mmol) and Cs₂CO₃ (23.4 g, 71.9 mmol) in degassed 1,4-dioxane (150 mL) was heated to 75° C. for 18 h and then cooled to rt. The mixture was filtered through Celite and the filter cake washed with EtOAc (3×150 mL). The filtrate was concentrated under reduced pressure and the residue was purified by column chromatography on silica gel using a gradient of 0-40% EtOAc in hexanes to afford title compound tert-butyl 4-(3-(2,6-bis(benzyloxy)pyridin-3-yl)-1-methyl-1H-indazol-6-yl)piperazine-1-carboxylate (35.6 g, 98% yield) as a solid. MS (ESI) [M+H]⁺ 607.5.

tert-Butyl 4-(3-(2,6-dioxopiperidin-3-yl)-1-methyl-1H-indazol-6-yl)piperazine-1-carboxylate. A mixture of tert-butyl 4-[3-(2,6-dibenzyloxy-3-pyridyl)-1-methyl-indazol-6-yl]piperazine-1-carboxylate (35.6 g, 58.8 mmol) and Pearlman's catalyst (8.90 g, 25 wt. % loading) in EtOH (300 mL) and THF (300 mL) was subjected to hydrogenation (1 atm) at 50° C. for 10 h. The mixture was filtered through Celite and the filter cake washed with a 1:1 mixture of MeCN and MeOH (4×250 mL). The filtrate was concentrated under reduced pressure and the residue was purified by column chromatography on silica gel using a gradient of 0-100% EtOAc in hexanes to afford title compound tert-butyl 4-(3-(2,6-dioxopiperidin-3-yl)-1-methyl-1H-indazol-6-yl)piperazine-1-carboxylate (21.0 g, 84% yield) as a solid. MS (ESI) [M+H]⁺ 428.3.

3-(1-Methyl-6-(piperazin-1-yl)-1H-indazol-3-yl)piperidine-2,6-dione. To a solution of tert-butyl 4-[3-(2,6-dioxo-3-piperidyl)-1-methyl-indazol-6-yl]piperazine-1-carboxylate (21.0 g, 49.1 mmol) in 1,4-dioxane (150 mL) was added 4N HCl in 1,4-dioxane (98.2 mL, 393 mmol) and the reaction mixture was stirred at rt for 20 h. Et₂O (250 mL) was added and the precipitate was collected by filtration, washed with Et₂O (3×30 mL), then dried under vacuum and lyophilized to afford title compound 3-(1-methyl-6-(piperazin-1-yl)-1H-indazol-3-yl)piperidine-2,6-dione (17.6 g, 98% yield) as a solid. MS (ESI) [M+H]) 328.2. ¹H NMR (500 MHz, DMSO-d₆) δ 10.85 (s, 1H), 9.46 (s, 2H), 7.56 (d, J=8.9 Hz, 1H), 7.04-6.90 (m, 2H), 4.28 (dd, J=9.4, 5.0 Hz, 1H), 3.92 (s, 3H), 3.53-3.41 (m, 4H), 3.23 (s, 4H), 2.73-2.55 (m, 2H), 2.39-2.26 (m, 1H), 2.23-2.08 (m, 1H).

### Example 1.4. Synthesis of intermediate 3-(1-Methyl-7-(piperazin-1-yl)-1H-indazol-3-yl)piperidine-2,6-dione

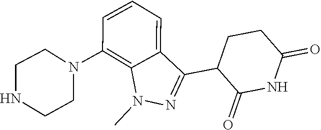 [SMILES: Cn1nc(C2CCC(=O)NC2=O)c2cccc(N3CCNCC3)c21]

7-Bromo-3-iodo-1-methyl-1H-indazole. Three batches carried out: to a solution of 7-bromo-3-iodo-1H-indazole (480 g, 1.49 mol, 1 equiv) in THF (2.4 L) at 0° C. was added portion-wise t-BuOK (334 g, 2.97 mol, 2 equiv). After the addition, the suspension was stirred at 0° C. for 1 h. Then a solution of CH₃I (422 g, 2.97 mol, 185 mL, 2 equiv) in THF (400 mL) was added dropwise to the cooled (0° C.) reaction mixture. The suspension was then stirred at 25° C. for 3 h. TLC (PE/EtOAc=5/1, Rf=0.5) showed the reaction was completed. The three reaction mixtures were combined and the resulting suspension was poured into water (10 L) and stirred for 10 min. The aqueous phase was extracted with EtOAc (5.0 L, then 3.0 L). The combined organic phase was washed with brine (3.0 L), dried with anhydrous Na₂SO₄, filtered and concentrated in vacuo. The crude was purified by column chromatography on silica gel (PE/EtOAc=25/1, 5/1) to give 7-bromo-3-iodo-1-methyl-1H-indazole (900 g, 2.67 mol, 60% yield) as a yellow solid. ¹H NMR (400 MHz, DMSO-d₆) δ ppm 7.72 (d, J=8.4 Hz, 1H), 7.49 (d, J=7.2 Hz, 1H), 7.11 (t, J=7.6 Hz, 1H), 4.34 (s, 3H).

3-(2,6-Bis(benzyloxy)pyridin-3-yl)-7-bromo-1-methyl-1H-indazole. Three batches carried out: to a solution of 7-bromo-3-iodo-1-methyl-1H-indazole (313 g, 929 mmol, 1 equiv) in 1,4-dioxane (2.0 L) and H₂O (1.0 L) was added (2,6-bis(benzyloxy)pyridin-3-yl)boronic acid (389 g, 929 mmol, 80% purity, 1 eq), K₃PO₄ (493 g, 2.32 mol, 2.5 equiv.) and Pd(PPh₃)₄(21.5 g, 18.6 mmol, 0.02 equiv.). Then the suspension was purged with N₂ three times and stirred at 90° C. for 12 h. The three batches were combined for work up and then the reaction mixture was poured into water (10 L) and stirred for 10 min. The aqueous phase was extracted with EtOAc (5 L, then 3 L). The combined organic phase was washed with brine (3 L), dried with anhydrous Na₂SO₄, filtered and concentrated in vacuo. The crude was purified by column chromatography on silica gel (PE/EtOAc=25/1, 5/1). The residue was triturated with PE/EtOAc (2/1) at 25° C. for 3 h, then the solids were collected by vacuum filtration to give 3-(2,6-bis(benzyloxy)pyridin-3-yl)-7-bromo-1-methyl-1H-indazole (920 g, 63% yield) as an off-white solid. ¹H NMR (400 MHz, DMSO-d₆) δ ppm 7.86 (d, J=8.4 Hz, 1H), 7.54 (dd, J=8.0, 0.8 Hz, 1H), 7.37 (dd, J=7.2, 0.8 Hz, 1H), 7.33 (m, 2H), 7.28 (m, 8H), 6.94 (t, J=7.6 Hz, 1H), 6.60 (d, J=7.6 Hz, 1H), 5.43 (s, 4H), 4.36 (s, 3H).

tert-Butyl 4-(3-(2,6-bis(benzyloxy)pyridin-3-yl)-1-methyl-1H-indazol-7-yl)piperazine-1-carboxylate. To a solution of 3-(2,6-bis(benzyloxy)pyridin-3-yl)-7-bromo-1-methyl-1H-indazole (100 g, 200 mmol,) and tert-butyl piperazine-1-carboxylate (55.8 g, 300 mmol) in 1,4-dioxane (700 mL) was added Cs₂CO₃ (130 g, 400 mmol), RuPhos (18.6 g, 40 mmol) and Pd₂(dba)₃(18.3 g, 20 mmol), then the suspension was purged with N₂ three times and stirred at 110° C. for 12 h. TLC (PE/EtOAc=3/1, R_{f}=0.6) showed the reaction was completed. The reaction was cooled to 20° C. and filtered through a pad of Celite. The filtrate was concentrated under vacuum and the residue was purified by silica gel chromatography (100-200 mesh silica gel, PE/EtOAc=20/1, 3/1) to give the product. The product was further purified by trituration with PE/EtOAc=(2/1, 200 mL) for 1 h. The solid was collected by filtration and dried under vacuum to afford tert-Butyl 4-(3-(2,6-bis(benzyloxy)pyridin-3-yl)-1-methyl-1H-indazol-7-yl)piperazine-1-carboxylate (84 g, 133 mmol, 67% yield, 96% purity) as a yellow solid. ¹H NMR (400 MHz DMSO-d₆) h ppm 7.85 (d, J=8.4 Hz, 1H), 7.27-7.45 (m, 12H), 6.96-6.99 (m, 2H), 6.53 (d, J=8.0 Hz, 1H), 5.47 (s, 2H), 5.40 (s, 2H), 4.41 (s, 3H), 4.10-4.16 (m, 2H), 3.20-3.23 (m, 4H), 2.84-2.89 (m, 2H), 1.51 (s, 9H).

tert-Butyl 4-(3-(2,6-dioxopiperidin-3-yl)-1-methyl-1H-indazol-7-yl)piperazine-1-carboxylate. To a suspension of tert-butyl 4-(3-(2,6-bis(benzyloxy)pyridin-3-yl)-1-methyl-1H-indazol-7-yl)piperazine-1-carboxylate (42 g, 69.3 mmol) and AcOH (4.16 g, 69.3 mmol, 3.97 mL) in THF (210 mL) and EtOH (210 mL) was added 10% Pd/C (8.0 g) and 20% Pd(OH)₂ (8.0 g, 57 mmol), then the black suspension was purged with H₂ three times and stirred at 50° C. under 50 psi for 12 h. The suspension was filtered through a pad of celite and the filter cake was washed with hot THF (2 L). The filtrate was concentrated under vacuum at 45° C. to get the crude product. The crude material was purified by silica gel chromatography (100-200 mesh silica gel, DCM/MeOH=0/1, 10/1) to give a solid. The solid was further triturated with MTBE (50 mL) for 1 h. The solid was collected by filtration and dried under vacuum. tert-butyl 4-(3-(2,6-dioxopiperidin-3-yl)-1-methyl-1H-indazol-7-yl)piperazine-1-carboxylate (18.6 g, 41.7 mmol, 30% yield, 96% purity) was obtained as a blue solid. MS (ESI) [M+H]⁺ 428.4.

3-(1-Methyl-7-(piperazin-1-yl)-1H-indazol-3-yl)piperidine-2,6-dione. To a solution of tert-Butyl 4-(3-(2,6-dioxopiperidin-3-yl)-1-methyl-1H-indazol-7-yl)piperazine-1-carboxylate (18.6 g, 43.5 mmol, 1 eq) in DCM (420 mL) was added HCl/EtOAc (4 M, 93.0 mL), then the suspension was stirred at 20° C. for 2 h. The solid was collected by filtration and dried under vacuum at 45° C. for 2 h. The solid was suspended in MeCN (100 mL) and dried under vacuum at 45° C. for 2 h. The operation was repeated two more times. 3-(1-Methyl-7-(piperazin-1-yl)-1H-indazol-3-yl)piperidine-2,6-dione (16.5 g, 41.2 mmol, 95% yield, 2HCl) was obtained as a light blue solid. ¹H NMR (400 MHz, DMSO-d₆) δ ppm 10.87 (s, 1H), 9.49-9.58 (m, 2H), 7.45 (d, J=6.8 Hz, 1H), 7.03-7.06 (m, 2H), 4.33-4.37 (m, 1H), 4.24 (s, 3H), 3.15-3.44 (m, 8H), 2.60-2.67 (m, 2H), 2.31-2.50 (m, 1H), 2.14-2.18 (m, 1H).

### Example 1.5. Synthesis of N-[trans-4-[[4-[3-(2,6-dioxo-3-piperidyl)-1-methyl-indazol-6-yl]piperazin-1-yl]methyl]cyclohexyl]-3-fluoro-4-[4-(4-oxo-1,5,6,7-tetrahydropyrrolo[3,2-c]pyridin-2-yl)-2-pyridyl]benzamide

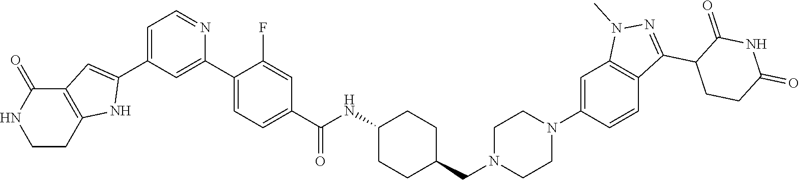 [SMILES: Cn1nc(C2CCC(=O)NC2=O)c2ccc(N3CCN(C[C@H]4CC[C@H](NC(=O)c5ccc(-c6cc(-c7cc8c([nH]7)CCNC8=O)ccn6)c(F)c5)CC4)CC3)cc21]

Methyl 3-fluoro-4-(4-(4-oxo-4,5,6,7-tetrahydro-1H-pyrrolo[3,2-c]pyridin-2-yl)pyridin-2-yl)benzoate. 2-(2-chloro-4-pyridyl)-1,5,6,7-tetrahydropyrrolo[3,2-c]pyridin-4-one (5.8 g, 23.4 mmol), (2-fluoro-4-methoxycarbonyl-phenyl)boronic acid (Int. A-2) (5.8 g, 29.3 mmol), and Pd(dppf)C12 (1.71 g, 2.34 mmol) in 1,4-dioxane (174 mL) and water (17.4 mL) was degassed and then heated to 90° C. for 16 hours under N₂. LCMS showed the starting material was consumed completely. The reaction mixture was poured into H₂O (50 mL). The mixture was extracted with EtOAc (2×30 mL). The organic phase was washed with brine (30 mL), dried over anhydrous Na₂SO₄, concentrated in vacuo to give crude material, which was purified by prep-HPLC to give methyl 3-fluoro-4-[4-(4-oxo-1,5,6,7-tetrahydropyrrolo[3,2-c]pyridine-2-yl)-2-pyridyl]benzoate (6.5 g, 76% yield) as a orange oil. MS (EST) [M+H]⁺ 366.1. ¹H NMR (400 MHz, DMSO-d₆) δ ppm 12.06 (s, 1H), 8.64 (d, J=5.2 Hz, 1H), 8.07-7.68 (m, 5H), 7.09 (d, J=3.6 Hz, 1H), 3.92 (s, 3H), 3.44-3.41 (m, 2H), 2.88-2.85 (m, 2H).

3-Fluoro-4-(4-(4-oxo-4,5,6,7-tetrahydro-1H-pyrrolo[3,2-c]pyridin-2-yl)pyridin-2-yl)benzoic acid. To a solution of methyl 3-fluoro-4-[4-(4-oxo-1,5,6,7-tetrahydropyrrolo[3,2-c]pyridin-2-yl)-2-pyridyl]benzoate (6.5 g, 17.8 mmol) in THF (70 mL) and water (35 mL) was added LiOH·H₂O (2.24 g, 53.4 mmol). The reaction mixture was stirred at 20° C. for 16 hours. LCMS showed a major peak that is consistent with product. The reaction mixture was concentrated. The residue was adjusted to pH=3 by HCl (2N), the formed solid was collected by vacuum filtration and the cake was dried to give 3-fluoro-4-[4-(4-oxo-1,5,6,7-tetrahydropyrrolo[3,2-c]pyridin-2-yl)-2-pyridyl]benzoic acid (5.5 g, 88% yield) as a yellow solid. MS (ESI) [M+H]⁺ 352.2. ¹H NMR (400 MHz, DMSO-d₆) δ ppm 12.05 (s, 1H), 8.63 (d, J=5.2 Hz, 1H), 8.06-7.87 (m, 4H), 7.69-7.68 (m, 1H), 7.08 (s, 2H), 3.91 (s, 3H), 3.44-3.40 (m, 2H), 2.87-2.76 (m, 2H).

tert-Butyl N-[trans-4-[[4-[3-(2,6-Dioxo-3-piperidyl)-1-methyl-indazol-6-yl]piperazin-1-yl]methyl]cyclohexyl]carbamate. To a solution of 3-(1-methyl-6-piperazin-1-yl-indazol-3-yl)piperidine-2,6-dione; hydrochloride (660 mg, 1.81 mmol) in CH₂Cl₂ (10 mL) was added DIPEA (1.44 mL, 8.25 mmol). The reaction mixture was stirred at room temperature for 10 minutes. Then, tert-butyl N-(4-formylcyclohexyl)carbamate (374.8 mg, 1.65 mmol, 1.1 equiv) was added followed by NaBH(OAc)3 (873.76 mg, 4.12 mmol). The reaction was stirred at room temperature overnight. LCMS showed complete conversion. The solvent was evaporated under reduced pressure and the crude product was purified by reverse phase chromatography (Isco, 100 g, MeOH/H₂O+0.1% FA, 5% MeOH for 5 CV, then 10 CV 5 to 50% MeOH and 50 to 100% MeOH on 5 CV) to give tert-butyl N-[4-[[4-[3-(2,6-dioxo-3-piperidyl)-1-methyl-indazol-6-yl]piperazin-1-yl]methyl]cyclohexyl]carbamate; formic acid (735 mg, 76%) as an off-white solid. MS (ESI) [M−HCOOH+H]+ 539.4. ¹H NMR (400 MHz, DMSO-d6) δ ppm 0.82-0.98 (m, 2H), 1.06-1.22 (m, 2H), 1.37 (s, 9H), 1.42-1.50 (m, 1H), 1.78 (br d, J=10.5 Hz, 4H), 2.11-2.22 (m, 3H), 2.24-2.36 (m, 1H), 2.52-2.69 (m, 5H), 3.22 (br s, 6H), 3.89 (s, 3H), 4.25 (dd, J=9.2, 5.0 Hz, 1H), 6.70 (br d, J=7.6 Hz, 1H), 6.83 (d, J=1.5 Hz, 1H), 6.91 (dd, J=9.0, 2.0 Hz, 1H), 7.49 (d, J=9.0 Hz, 1H), 8.13 (s, 1H), 10.84 (s, 1H).

3-[6-[4-[(4-Aminocyclohexyl)methyl]piperazin-1-yl]-1-methyl-indazol-3-yl]piperidine-2,6-dione. To a solution of tert-butyl N-[4-[[4-[3-(2,6-dioxo-3-piperidyl)-1-methyl-indazol-6-yl]piperazin-1-yl]methyl]cyclohexyl]carbamate (735 mg, 1.36 mmol) in CH₂Cl₂(5 mL) was added 4.0 M HCl in 1,4-dioxane (5 mL, 20.47 mmol) and the reaction mixture was stirred at room temperature overnight. The solvent was removed under reduced pressure. MeCN was added to the residue and the solvent removed under reduced pressure to remove residual HCL. This procedure was repeated 2 more times with MeCN and 2 times with CH₂Cl₂ to give 3-[6-[4-[(4-aminocyclohexyl)methyl]piperazin-1-yl]-1-methyl-indazol-3-yl]piperidine-2,6-dione; dihydrochloride (700 mg, quantitative yield) as an off-white solid. MS (ESI) [M+H]⁺=439.2. ¹H NMR (400 MHz, DMSO-d₆) δ ppm 1.01-1.15 (m, 2H), 1.37 (br d, J=10.5 Hz, 2H), 1.81 (br d, J=2.7 Hz, 1H), 1.97 (br d, J=11.0 Hz, 4H), 2.12-2.21 (m, 1H), 2.31 (dt, J=13.8, 4.5 Hz, 1H), 2.55-2.69 (m, 2H), 2.88-3.04 (m, 3H), 3.09-3.22 (m, 2H), 3.38 (br t, J=12.1 Hz, 2H), 3.60 (br s, 1H), 3.64-3.73 (m, 1H), 3.87 (br d, J=13.0 Hz, 2H), 3.92 (s, 3H), 4.28 (br dd, J=9.4, 5.0 Hz, 1H), 6.93-7.00 (m, 2H), 7.52-7.60 (m, 1H), 8.09 (br s, 3H), 8.14 (s, 1H), 10.68 (br d, J=1.5 Hz, 1H), 10.86 (s, 1H).

N-[4-[[4-[3-(2,6-dioxo-3-piperidyl)-1-methyl-indazol-6-yl]piperazin-1-yl]methyl]cyclohexyl]-3-fluoro-4-[4-(4-oxo-1,5,6,7-tetrahydropyrrolo[3,2-c]pyridin-2-yl)-2-pyridyl]benzamide. To a solution of 3-[6-[4-[(4-aminocyclohexyl)methyl]piperazin-1-yl]-1-methyl-indazol-3-yl]piperidine-2,6-dione; dihydrochloride (360.3 mg, 0.70 mmol) and 3-fluoro-4-[4-(4-oxo-1,5,6,7-tetrahydropyrrolo[3,2-c]pyridin-2-yl)-2-pyridyl]benzoic acid (225 mg, 0.64 mmol) in DMF (3 mL) was added DIPEA (0.56 mL, 3.2 mmol) at room temperature. After 10 min, HATU (365.26 mg, 0.9600 mmol) was added and the reaction mixture was stirred at room temperature. After 2 h, LCMS showed full conversion. The mixture was directly loaded onto a C18 column and purified by reverse phase chromatography (Isco, 100 g, 5 to 100% MeCN/water+0.1% FA on 25 CV) to afford clean and fractions contaminated with PF₆. The fractions containing product and PF₆ were evaporated and purified again using the same conditions. After the second purification the clean fractions were combined and, after lyophilization, afforded N-[4-[[4-[3-(2,6-dioxo-3-piperidyl)-1-methyl-indazol-6-yl]piperazin-1-yl]methyl]cyclohexyl]-3-fluoro-4-[4-(4-oxo-1,5,6,7-tetrahydropyrrolo[3,2-c]pyridin-2-yl)-2-pyridyl]benzamide as a partial formic acid salt (1:0.38) (197 mg, 40% yield) and as a yellow solid. MS (ESI) [M+H]⁺=772.5. ¹H NMR (400 MHz, DMSO-d₆) δ ppm 0.94-1.10 (m, 2H), 1.31-1.47 (m, 2H), 1.48-1.60 (m, 1H), 1.83-1.97 (m, 4H), 2.10-2.23 (m, 3H), 2.27-2.36 (m, 1H), 2.52-2.69 (m, 6H), 2.86 (t, J=6.8 Hz, 2H), 3.23 (br s, 4H), 3.42 (td, J=6.4, 1.7 Hz, 2H), 3.78 (br dd, J=7.7, 4.3 Hz, 1H), 3.89 (s, 3H), 4.26 (dd, J=8.9, 5.3 Hz, 1H), 6.84 (s, 1H), 6.92 (br d, J=9.5 Hz, 1H), 7.04-7.11 (m, 2H), 7.50 (d, J=8.8 Hz, 1H), 7.66 (dd, J=5.4, 1.5 Hz, 1H), 7.77-7.89 (m, 2H), 7.95-8.06 (m, 2H), 8.36-8.47 (m, 1H), 8.63 (d, J=5.1 Hz, 1H), 10.85 (s, 1H), 12.04 (br s, 1H). ¹⁹F NMR (377 MHz, DMSO-d₆) δ ppm −116.50-−116.22 (m, 1F).

### Example 1.6. Synthesis of N-(trans-4-((4-(4-(2,6-Dioxopiperidin-3-yl)phenyl)piperazin-1-yl)methyl)cyclohexyl)-3-fluoro-4-(4-(4-oxo-4,5,6,7-tetrahydro-1H-pyrrolo[3,2-c]pyridin-2-yl)pyridin-2-yl)benzamide

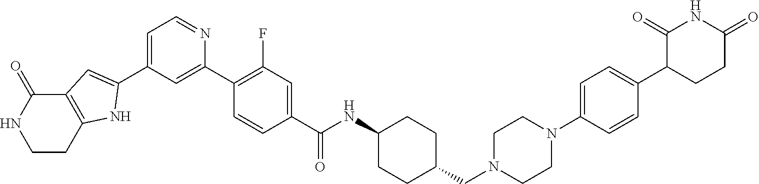 [SMILES: O=C1CCC(c2ccc(N3CCN(C[C@H]4CC[C@H](NC(=O)c5ccc(-c6cc(-c7cc8c([nH]7)CCNC8=O)ccn6)c(F)c5)CC4)CC3)cc2)C(=O)N1]

tert-Butyl-N-[4-[[4-[4-(2,6-dioxo-3-piperidyl)phenyl]piperazin-1-yl]methyl]cyclohexyl]carbamate. To a stirred solution of 3-(4-piperazin-1-ylphenyl)piperidine-2,6-dione (200 mg, 0.73 mmol) and tert-butyl N-(4-formylcyclohexyl)carbamate (249 mg, 1.1 mmol) and TEA (0.2 mL, 1.15 mmol) and NaBH₃CN (92 mg, 1.44 mmol) was added MeOH (2 mL). The mixture was stirred at rt for 3 h. LC/MS showed the reaction afforded a peak consistent with desired product. The reaction was then quenched with water (30 mL) and extracted with EtOAc (3×30 mL). The combined organic layer was dried over Na₂SO₄, filtered, and concentrated. The residue was purified by silica gel column chromatography and using PE/EtOAc (0-70%) as eluent, to afford tert-butyl N-[4-[[4-[4-(2,6-dioxo-3-piperidyl)phenyl]piperazin-1-yl]methyl]cyclohexyl]carbamate (240 mg, 68% yield) as a white solid. MS (ESI) m/z 485.4 [M+H]⁺.

3-[4-[4-[(4-Aminocyclohexyl)methyl]piperazin-1-yl]phenyl]piperidine-2,6-dione. To a stirred solution of tert-butyl N-[4-[[4-[4-(2,6-dioxo-3-piperidyl)phenyl]piperazin-1-yl]methyl]cyclohexyl]carbamate (240 mg, 0.50 mmol) was added HCl in EtOAc (10 mL, 0.50 M). The mixture was stirred at rt for 1 h. LC/MS showed the reaction afforded the expecting product. After filtration, a crude product, 3-[4-[4-[(4-aminocyclohexyl)methyl]piperazin-1-yl]phenyl]piperidine-2,6-dione (180 mg, 94.5%) was obtained as a white solid. MS (ESI) m/z 385.1 [M+H]⁺.

N-[4-[[4-[4-(2,6-Dioxo-3-piperidyl)phenyl]piperazin-1-yl]methyl]cyclohexyl]-3-fluoro-4-[4-(4-oxo-1,5,6,7-tetrahydropyrrolo[3,2-c]pyridin-2-yl)-2-pyridyl]benzamide. To a stirred solution of 3-[4-[4-[(4-aminocyclohexyl)methyl]piperazin-1-yl]phenyl]piperidine-2,6-dione (131 mg, 0.34 mmol) and 3-fluoro-4-[4-(4-oxo-1,5,6,7-tetrahydropyrrolo[3,2-c]pyridin-2-yl)-2-pyridyl]benzoic acid (100 mg, 0.28 mmol) and DIEA (0.12 mL, 1.43 mmol) and HATU (217 mg, 0.57 mmol) was added DMF (1 mL). The mixture was stirred at rt for 2 h. LC/MS showed the reaction afforded good conversion. The reaction was then quenched with water (100 mL) and extracted with EtOAc (3×100 mL). The combined organic layer was dried over Na₂SO₄, filtered, and concentrated. The residue was purified by prep-HPLC (Column: Atlantis Prep T3 OBD, 19×150 mm, 5 mm. Mobile Phase A: Water (0.05% TFA), Mobile Phase B: CH₃CN; Flow rate: 25 mL/min. Detection UV @254/210 nm) to afford N-[4-[[4-[4-(2,6-dioxo-3-piperidyl)phenyl]piperazin-1-yl]methyl]cyclohexyl]-3-fluoro-4-[4-(4-oxo-1,5,6,7-tetrahydropyrrolo[3,2-c]pyridin-2-yl)-2-pyridyl]benzamide (31.9 mg, 15% yield) as a yellow solid. MS (ESI) m/z 718.6 [M+H]^{+ 1}H NMR (300 MHz, DMSO-d₆) δ ppm 8.70 (d, J=5.7 Hz, 1H), 8.15 (s, 1H), 8.06-7.95 (m, 1H), 7.93-7.79 (m, 3H), 7.27 (s, 1H), 7.14 (d, J=8.7 Hz, 2H), 6.99 (d, J=8.5 Hz, 2H), 3.93-3.73 (m, 4H), 3.63 (d, J=11.3 Hz, 2H), 3.49-3.41 (m, 3H), 3.30-3.04 (m, 6H), 2.96-2.85 (m, 2H), 2.74-2.61 (m, 1H), 2.23-1.74 (m, 7H), 1.54-1.36 (m, 2H), 1.20-1.14 (m, 2H). ¹⁹F NMR (377 MHz, DMSO-d₆) δ ppm −115.8 (s, 1F).

### Example 1.7. Synthesis of N-(trans-4-((4-(3-(2,6-Dioxopiperidin-3-yl)phenyl)piperazin-1-yl)methyl)cyclohexyl)-3-fluoro-4-(4-(4-oxo-4,5,6,7-tetrahydro-1H-pyrrolo[3,2-c]pyridin-2-yl)pyridin-2-yl)benzamide

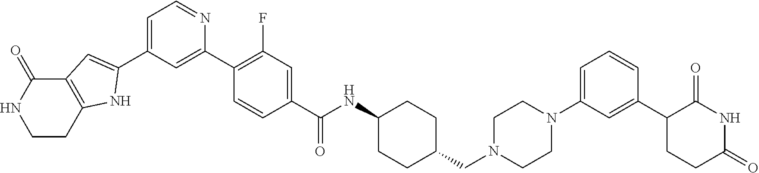 [SMILES: O=C1CCC(c2cccc(N3CCN(C[C@H]4CC[C@H](NC(=O)c5ccc(-c6cc(-c7cc8c([nH]7)CCNC8=O)ccn6)c(F)c5)CC4)CC3)c2)C(=O)N1]

3-(3-Bromophenyl)-1-[(4-methoxyphenyl)methyl]piperidine-2,6-dione. To a stirred solution of 3-(3-bromophenyl)piperidine-2,6-dione (300 mg, 1.12 mmol) in DMF (5 mL) was added K₂CO₃ (226 mg). The mixture was stirred at 40° C. overnight. The reaction was monitored by LC/MS. The reaction was quenched with H₂O (50 mL) and extracted with EtOAc (3×50 mL). The combined organic phase was dried over Na₂SO₄, filtered and concentrated. The residue was purified by silica gel column chromatography and using hexane/EtOAc (0-50%) as eluent to afford 3-(3-bromophenyl)-1-[(4-methoxyphenyl)methyl]piperidine-2,6-dione (270 mg, 62% yield) as a yellow solid. MS (ESI) m/z 388.0 [M+H]⁺.

4-[3-[1-[(4-Methoxyphenyl)methyl]-2,6-dioxo-3-piperidyl]phenyl]piperazine-1-carboxylate. To a stirred solution of 3-(3-bromophenyl)-1-[(4-methoxyphenyl)methyl]piperidine-2,6-dione (270 mg, 0.70 mmol) in 1,4-dioxane (5 mL) was added Cs₂CO₃ (678 mg, 2.1 mmol), Cphos (50 mg, 0.11 mmol) and Pd₂(dba)₃ (60.0 mg, 0.065 mmol). The mixture was stirred at 80° C. overnight. The reaction was monitored by LC/MS. The reaction was quenched with H₂O (50 mL) and extracted with EtOAc (3×50 mL). The combined organic phase was dried over Na₂SO₄, filtered and concentrated. The residue was purified by silica gel column chromatography and using hexane/EtOAc (0-70%) as eluent to afford tert-butyl 4-[3-[1-[(4-methoxyphenyl)methyl]-2,6-dioxo-3-piperidyl]phenyl]piperazine-1-carboxylate (160 mg, 46% yield) as a yellow solid. MS (ESI) m/z 494.1 [M+H]⁺.

1-[(4-Methoxyphenyl)methyl]-3-(3-piperazin-1-ylphenyl)piperidine-2,6-dione. To a stirred solution of tert-butyl 4-[3-[1-[(4-methoxyphenyl)methyl]-2,6-dioxo-3-piperidyl]phenyl]piperazine-1-carboxylate (160 mg, 0.32 mmol) in 2 M HCl in EtOAc (10 mL). The mixture was stirred at rt for 2 h. The reaction was monitored by LC/MS. The solid was collected by vacuum filtration and air dried to afford 1-[(4-methoxyphenyl)methyl]-3-(3-piperazin-1-ylphenyl)piperidine-2,6-dione (180 mg, crude, quantitative yield) as a brown solid. MS (ESI) m/z 393.4 [M+H]⁺.

N-[4-[[4-[3-[1-[(4-Methoxyphenyl)methyl]-2,6-dioxo-3-piperidyl]phenyl]piperazin-1-yl]methyl]cyclohexyl]carbamate. To a stirred solution of 1-[(4-methoxyphenyl)methyl]-3-(3-piperazin-1-ylphenyl)piperidine-2,6-dione (60 mg, 0.15 mmol) and tert-butyl N-(4-formylcyclohexyl)carbamate (51 mg, 0.23 mmol) in MeOH (1 mL) was added ZnCl₂(2.0 M in 2-Me THF) (0.5 mL, 1 mmol) and NaBH₃CN (29 mg, 0.46 mmol). The mixture was stirred at 60° C. for 4 h. The reaction was monitored by LC/MS. The reaction was quenched with H₂O (50 mL) and extracted with EtOAc (3×50 mL). The combined organic phase was dried over Na₂SO₄, filtered and concentrated. The residue was purified by silica gel column chromatography and using hexane/EtOAc (0-50%) as eluent to afford tert-butyl N-[4-[[4-[3-[1-[(4-methoxyphenyl)methyl]-2,6-dioxo-3-piperidyl]phenyl]piperazin-1-yl]methyl]cyclohexyl]carbamate (80 mg, 86% yield) as a pink solid. MS (ESI) m/z 605.5 [M+H]⁺.

3-[3-[4-[(4-Aminocyclohexyl)methyl]piperazin-1-yl]phenyl]-1-[(4-methoxyphenyl)methyl]piperidine. To a stirred solution of tert-butyl N-[4-[[4-[3-[1-[(4-methoxyphenyl)methyl]-2,6-dioxo-3-piperidyl]phenyl]piperazin-1-yl]methyl]cyclohexyl]carbamate (160 mg, 0.26 mmol) in 2 M HCl in EtOAc (10 mL) at rt. The mixture was stirred at rt for 2 h. The reaction was monitored by LC/MS. The solid was filtered to afford 3-[3-[4-[(4-aminocyclohexyl)methyl]piperazin-1-yl]phenyl]-1-[(4-methoxyphenyl)methyl]piperidine-2,6-dione (130 mg, 97% yield) as a brown solid. MS (ESI) m/z 505.3 [M+H]⁺.

3-Fluoro-N-[4-[[4-[3-[1-[(4-methoxyphenyl)methyl]-2,6-dioxo-3-piperidyl]phenyl]piperazin-1-yl]methyl]cyclohexyl]-4-[4-(4-oxo-1,5,6,7-tetrahydropyrrolo[3,2-c]pyridin-2-yl)-2-pyridyl]benzamide. To a stirred a solution of 3-[3-[4-[(4-aminocyclohexyl)methyl]piperazin-1-yl]phenyl]-1-[(4-methoxyphenyl)methyl]piperidine-2,6-dione (143 mg, 0.28 mmol) and 3-fluoro-4-[4-(4-oxo-1,5,6,7-tetrahydropyrrolo[3,2-c]pyridin-2-yl)-2-pyridyl]benzoic acid (100 mg, 0.28 mmol) in DMF (2 mL) was added HATU (216 mg, 0.57 mmol) and DIEA (146 mg, 1.14 mmol). The mixture was stirred at rt for 2 h. The reaction was monitored by LC/MS. The reaction was quenched with H₂O (50 mL) and extracted with EtOAc (3×50 mL). The combined organic phase was dried over Na₂SO₄, filtered and concentrated. The residue was purified by silica gel column chromatography and using hexane/EtOAc (0-70%) as eluent to afford 3-fluoro-N-[4-[[4-[3-[1-[(4-methoxyphenyl)methyl]-2,6-dioxo-3-piperidyl]phenyl]piperazin-1-yl]methyl]cyclohexyl]-4-[4-(4-oxo-1,5,6,7-tetrahydropyrrolo[3,2-c]pyridin-2-yl)-2-pyridyl]benzamide (140 mg, 58% yield) as a green solid. MS (ESI) m/z 838.1 [M+H]⁺.

3-Fluoro-N-[4-[[4-[3-[1-[(4-methoxyphenyl)methyl]-2,6-dioxo-3-piperidyl]phenyl]piperazin-1-yl]methyl]cyclohexyl]-4-[4-(4-oxo-1,5,6,7-tetrahydropyrrolo[3,2-c]pyridin-2-yl)-2-pyridyl]benzamide. To a stirred solution of 3-fluoro-N-[4-[[4-[3-[1-[(4-methoxyphenyl)methyl]-2,6-dioxo-3-piperidyl]phenyl]piperazin-1-yl]methyl]cyclohexyl]-4-[4-(4-oxo-1,5,6,7-tetrahydropyrrolo[3,2-c]pyridin-2-yl)-2-pyridyl]benzamide (110 mg, 0.13 mmol) in TFA (5 mL) was added TfOH (0.5 mL, 5.66 mmol). The mixture was stirred at 70° C. for 3 h. The reaction was monitored by LCMS. The solvent was removed in vacuo and the residue was purified by prep-HPLC (Column: SunFire Cis OBD 19×150 mm. 5.0 mm. Mobile Phase A: Water (0.05% TFA). Mobile Phase B: CH₃CN; Flow rate: 25 mL/min; Detection UV@254/210 nm) to afford N-[4-[[4-[3-(2,6-dioxo-3-piperidyl)phenyl]piperazin-1-yl]methyl]cyclohexyl]-3-fluoro-4-[4-(4-oxo-1,5,6,7-tetrahydropyrrolo[3,2-c]pyridin-2-yl)-2-pyridyl]benzamide (7.2 mg, 7.5% yield, 0.01 mmol) as a yellow solid. MS (ESI) m/z 718.3 [M+H]~¹H NMR (400 MHz, DMSO-d₆) δ ppm 8.67 (d, J=5.6 Hz, 1H), 8.09-7.95 (m, 2H), 7.89-7.73 (m, 3H), 7.28-7.19 (m, 2H), 6.95-6.86 (m, 2H), 6.73 (d, J=7.6 Hz, 1H), 3.84-3.74 (m, 4H), 3.62 (d, J=11.6 Hz, 2H), 3.50-3.42 (m, 3H), 3.21-3.01 (m, 6H), 2.92-2.84 (m, 2H), 2.78-2.72 (m, 1H), 2.24-2.17 (m, 1H), 2.07-1.98 (m, 1H), 1.98-1.81 (m, 5H), 1.50-1.40 (m, 2H), 1.25-1.10 (m, 2H). ¹⁹F NMR (377 MHz, DMSO-d₆) δ ppm −115.8 (s, 1F).

### Example 1.8. Synthesis of N-(trans-4-(2-(4-(3-(2,6-Dioxopiperidin-3-yl)-1-methyl-1H-indazol-6-yl)piperazin-1-yl)ethyl)cyclohexyl)-3-fluoro-4-(4-(4-oxo-4,5,6,7-tetrahydro-1H-pyrrolo[3,2-c]pyridin-2-yl)pyridin-2-yl)benzamide (I-4)

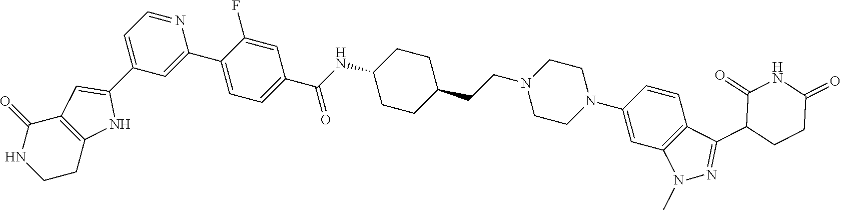 [SMILES: Cn1nc(C2CCC(=O)NC2=O)c2ccc(N3CCN(CC[C@H]4CC[C@H](NC(=O)c5ccc(-c6cc(-c7cc8c([nH]7)CCNC8=O)ccn6)c(F)c5)CC4)CC3)cc21]

tert-Butyl N-[4-[2-[4-[3-(2,6-dioxo-3-piperidyl)-1-methyl-indazol-6-yl]piperazin-1-yl]ethyl]cyclohexyl]carbamate. To a stirred solution of 3-(1-methyl-6-piperazin-1-yl-indazol-3-yl)piperidine-2,6-dione (150 mg, 0.46 mmol) and tert-butyl N-[4-(2-oxoethyl)cyclohexyl]carbamate (165 mg, 0.69 mmol) and NaBH(OAc)₃ (345 mg, 1.63 mmol) and TEA (0.11 mL, 0.65 mmol) was added DCE (5 mL, 0.46 mmol). The mixture was stirred at rt for 3 h. The reaction was monitored by LC/MS. The reaction was quenched with H₂O (30 mL) and extracted with EtOAc (3×30 mL). The combined organic phase was dried over Na₂SO₄, filtered and concentrated. The residue was purified by silica gel column chromatography and using hexane/EtOAc (0-70%) as eluent to give tert-butyl N-[4-[2-[4-[3-(2,6-dioxo-3-piperidyl)-1-methyl-indazol-6-yl]piperazin-1-yl]ethyl]cyclohexyl]carbamate (210 mg, 83% yield) as a yellow solid. MS (ESI) m/z 553.5 [M+H]⁻.

3-[6-[4-[2-(4-Aminocyclohexyl)ethyl]piperazin-1-yl]-1-methyl-indazol-3-yl]piperidine-2,6-dione. To a stirred solution of tert-butyl N-[4-[2-[4-[3-(2,6-dioxo-3-piperidyl)-1-methyl-indazol-6-yl]piperazin-1-yl]ethyl]cyclohexyl]carbamate (210 mg, 0.38 mmol) was added HCl (EtOAc) (10 mL, 0.38 mmol). The mixture was stirred at it for 1 h. The reaction was monitored by LCMS. The solid was collected by filtered to afford 3-[6-[4-[2-(4-aminocyclohexyl)ethyl]piperazin-1-yl]-1-methyl-indazol-3-yl]piperidine-2,6-dione (160 mg, 93% yield) as a yellow solid. MS (ESI) m/z 453.3 [M+H]⁺.

N-[4-[2-[4-[3-(2,6-Dioxo-3-piperidyl)-1-methyl-indazol-6-yl]piperazin-1-yl]ethyl]cyclohexyl]-3-fluoro-4-[4-(4-oxo-1,5,6,7-tetrahydropyrrolo[3,2-c]pyridin-2-yl)-2-pyridyl]benzamide. To a stirred solution of 3-[6-[4-[2-(4-aminocyclohexyl)ethyl]piperazin-1-yl]-1-methyl-indazol-3-yl]piperidine-2,6-dione (162 mg, 0.36 mmol) and 3-fluoro-4-[4-(4-oxo-1,5,6,7-tetrahydropyrrolo[3,2-c]pyridin-2-yl)-2-pyridyl]benzoic acid (70 mg, 0.20 mmol), HATU (151 mg, 0.40 mmol) and DIEA (0.09 mL, 1.13 mmol) was added DMF (4 mL). The mixture was stirred at rt for 2 h. The reaction was monitored by LC/MS. The resulting solution was purified by prep-HPLC (Column: Atlantis Prep T3 OBD, 19×150 mm, 5 mm; Mobile Phase A: Water (0.05% TFA), Mobile Phase B: CH₃CN. Flow rate: 25 mL/min. Detection UV@254/210 nm) to afford N-[4-[2-[4-[3-(2,6-dioxo-3-piperidyl)-1-methyl-indazol-6-yl]piperazin-1-yl]ethyl]cyclohexyl]-3-fluoro-4-[4-(4-oxo-1,5,6,7-tetrahydropyrrolo[3,2-c]pyridin-2-yl)-2-pyridyl]benzamide (41.9 mg, 25% yield, 0.053 mmol) as a yellow solid. MS (ESI) mal 786.3 [M+H]^{+ 1}H NMR (400 MHz, DMSO-d₆) δ ppm 8.67 (d, J=5.6 Hz, 1H), 8.10 (s, 1H), 8.03-7.95 (m, 1H), 7.89-7.81 (m, 2H), 7.81-7.74 (m, 1H), 7.58 (d, J=9.4 Hz, 1H), 7.21 (s, 1H), 6.98 (d, J=8.1 Hz, 2H), 4.33-4.25 (m, 1H), 4.03-3.98 (s, 2H), 3.93 (s, 3H), 3.85-3.75 (m, 1H), 3.63 (d, J=11.4 Hz, 2H), 3.43-3.39 (m, 2H), 3.26-3.15 (m, 4H), 3.11-3.01 (m, 2H), 2.93-2.85 (m, 2H), 2.68-2.59 (m, 2H), 2.31-2.24 (m, 1H), 2.21-2.12 (m, 1H), 1.92 (d, J=11.9 Hz, 2H), 1.83 (d, J=12.2 Hz, 2H), 1.65 (s, 2H), 1.46-1.25 (m, 3H), 1.19-1.03 (m, 2H). ¹⁹F NMR (377 MHz, DMSO-d₆) δ ppm −115.8 (s, 1F).

### Example 1.9. Synthesis of N-(trans-4-(2-(4-(3-(2,6-Dioxopiperidin-3-yl)phenyl)piperazin-1-yl)ethyl)cyclohexyl)-3-fluoro-4-(4-(4-oxo-4,5,6,7-tetrahydro-1H-pyrrolo[3,2-c]pyridin-2-yl)pyridin-2-yl)benzamide

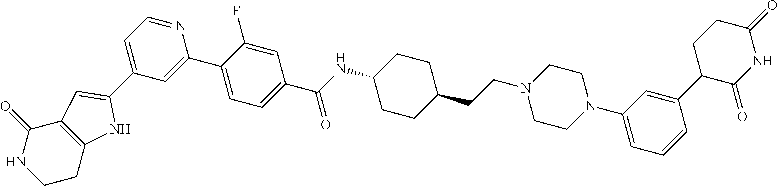 [SMILES: O=C1CCC(c2cccc(N3CCN(CC[C@H]4CC[C@H](NC(=O)c5ccc(-c6cc(-c7cc8c([nH]7)CCNC8=O)ccn6)c(F)c5)CC4)CC3)c2)C(=O)N1]

tert-Butyl-N-[4-[2-[4-[3-[1-[(4-methoxyphenyl)methyl]-2,6-dioxo-3-piperidyl]phenyl]piperazin-1-yl]ethyl]cyclohexyl]carbamate. To a stirred solution of 1-[(4-methoxyphenyl)methyl]-3-(3-piperazin-1-ylphenyl)piperidine-2,6-dione (180 mg, 0.46 mmol) and tert-butyl N-[4-(2-oxoethyl)cyclohexyl]carbamate (220 mg, 0.91 mmol) in MeOH (2 mL) was added ZnCl₂ (2.0 M in 2-MeTHF) (0.2 mL, 0.4 mmol) and NaBH₃CN (86 mg, 1.37 mmol). The mixture was stirred at 60° C. for 4 h. The reaction was monitored by LCMS. The reaction was quenched with H₂O (50 mL) and extracted with EtOAc (3×50 mL). The combined organic phase was dried over Na₂SO₄, filtered and concentrated. The residue was purified by silica gel column chromatography and using hexane/EtOAc (0-70%) as eluent to afford tert-butyl N-[4-[2-[4-[3-[1-[(4-methoxyphenyl)methyl]-2,6-dioxo-3-piperidyl]phenyl]piperazin-1-yl]ethyl]cyclohexyl]carbamate (180 mg, 64% yield) as a white solid. MS (ESI) m/z 619.5 [M+H]⁺.

3-[3-[4-[2-(4-Aminocyclohexyl)ethyl]piperazin-1-yl]phenyl]-1-[(4-methoxyphenyl)methyl]piperidine-2,6-dione. To a stirred solution of tert-butyl N-[4-[2-[4-[3-[1-[(4-methoxyphenyl)methyl]-2,6-dioxo-3-piperidyl]phenyl]piperazin-1-yl]ethyl]cyclohexyl]carbamate (150 mg, 0.24 mmol) in 2 M HCl in EtOAc (10 mL) at rt. The mixture was stirred at rt for 2 h. The reaction was monitored by LC/MS. The solvent was removed in vacuo to afford 3-[3-[4-[2-(4-aminocyclohexyl)ethyl]piperazin-1-yl]phenyl]-1-[(4-methoxyphenyl)methyl]piperidine-2,6-dione (120 mg, 95% yield) as a brown solid. MS (ESI) m/z 519.4 [M+H]⁺.

3-Fluoro-N-[4-[2-[4-[3-[1-[(4-methoxyphenyl)methyl]-2,6-dioxo-3-piperidyl]phenyl]piperazin-1-yl]ethyl]cyclohexyl]-4-[4-(4-oxo-1,5,6,7-tetrahydropyrrolo[3,2-c]pyridin-2-yl)-2-pyridyl]benzamide. To a stirred solution of 3-[3-[4-[2-(4-aminocyclohexyl)ethyl]piperazin-1-yl]phenyl]-1-[(4-methoxyphenyl)methyl]piperidine-2,6-dione (120 mg, 0.23 mmol) and 3-fluoro-4-[4-(4-oxo-1,5,6,7-tetrahydropyrrolo[3,2-c]pyridin-2-yl)-2-pyridyl]benzoic acid (81 mg, 0.23 mmol) in DMF (2 mL) was added HATU (175 mg, 0.46 mmol) and DIEA (89 mg, 0.69 mmol). The mixture was stirred at rt for 2 h. The reaction was monitored by LCMS. The reaction was quenched with H₂O (50 mL) and extracted with EtOAc (3×50 mL). The combined organic phase was dried over Na₂SO₄, filtered and concentrated. The residue was purified by silica gel column chromatography and using hexane/EtOAc (0-70%) as eluent to afford 3-fluoro-N-[4-[2-[4-[3-[1-[(4-methoxyphenyl)methyl]-2,6-dioxo-3-piperidyl]phenyl]piperazin-1-yl]ethyl]cyclohexyl]-4-[4-(4-oxo-1,5,6,7-tetrahydropyrrolo[3,2-c]pyridin-2-yl)-2-pyridyl]benzamide (100 mg, 51% yield) as a green solid. MS (ESI) m/z 852.6 [M+H]⁺.

N-[4-[2-[4-[3-(2,6-Dioxo-3-piperidyl)phenyl]piperazin-1-yl]ethyl]cyclohexyl]-3-fluoro-4-[4-(4-oxo-1,5,6,7-tetrahydropyrrolo[3,2-c]pyridin-2-yl)-2-pyridyl]benzamide. To a stirred solution of 3-fluoro-N-[4-[2-[4-[3-[1-[(4-methoxyphenyl)methyl]-2,6-dioxo-3-piperidyl]phenyl]piperazin-1-yl]ethyl]cyclohexyl]-4-[4-(4-oxo-1,5,6,7-tetrahydropyrrolo[3,2-c]pyridin-2-yl)-2-pyridyl]benzamide (120 mg, 0.1400 mmol) in TFA (5 mL) was added TfOH (0.5 mL) at rt. The mixture was stirred at 70° C. for 3 h. The reaction was monitored by LCMS. The solvent was removed in vacuo and the residue was purified by prep-HPLC (Column: SunFire C₁₈ OBD, 19×150 mm 5 mm. Mobile Phase A: Water (0.05% TFA), Mobile Phase B: CH₃CN. Flow rate: 25 mL/min. Detection UV @254/210 nm) to afford N-[4-[2-[4-[3-(2,6-dioxo-3-piperidyl)phenyl]piperazin-1-yl]ethyl]cyclohexyl]-3-fluoro-4-[4-(4-oxo-1,5,6,7-tetrahydropyrrolo[3,2-c]pyridin-2-yl)-2-pyridyl]benzamide (10.7 mg, 10% yield, 0.015 mmol) as a yellow solid. MS (ESI) m/z 732.4 [M+H]^{+ 1}H NMR (300 MHz, DMSO-d₆) δ ppm 8.66 (d, J=5.7 Hz, 1H), 8.15-8.06 (m, 1H), 8.02-7.91 (m, 1H), 7.90-7.76 (m, 3H), 7.28-7.17 (m, 2H), 6.95-6.84 (m, 2H), 6.73 (d, J=7.6 Hz, 1H), 3.88-3.73 (m, 4H), 3.58-3.52 (m, 2H), 3.49-3.38 (m, 2H), 3.26-3.06 (m, 4H), 3.03-2.84 (m, 4H), 2.74-2.60 (m, 1H), 2.48-2.44 (m, 1H), 2.26-2.12 (m, 1H), 2.08-1.96 (m, 1H), 1.90-1.73 (m, 4H), 1.62 (s, 2H), 1.47-1.29 (m, 3H), 1.19-1.01 (m, 2H). ¹⁹F NMR (377 MHz, DMSO-d₆) δ ppm −115.8 (s, 1F).

### Example 1.10. Synthesis of N-(trans-4-(2-(4-(4-(2,6-Dioxopiperidin-3-yl)phenyl)piperazin-1-yl)ethyl)cyclohexyl)-3-fluoro-4-(4-(4-oxo-4,5,6,7-tetrahydro-1H-pyrrolo[3,2-c]pyridin-2-yl)pyridin-2-yl)benzamide

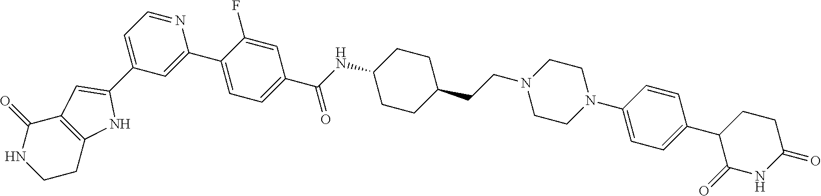 [SMILES: O=C1CCC(c2ccc(N3CCN(CC[C@H]4CC[C@H](NC(=O)c5ccc(-c6cc(-c7cc8c([nH]7)CCNC8=O)ccn6)c(F)c5)CC4)CC3)cc2)C(=O)N1]

tert-Butyl (trans-4-(2-(4-(4-(2,6-dioxopiperidin-3-yl)phenyl)piperazin-1-yl)ethyl)cyclohexyl)carbamate. To a stirred solution of 3-(4-piperazin-1-ylphenyl)piperidine-2,6-dione (150 mg, 0.55 mmol) and tert-butyl-N-[4-(2-oxoethyl)cyclohexyl]carbamate (264.9 mg, 1.1 mmol) in MeOH (5 mL) was added ZnCl₂ (2.0 M in 2-Me THF, 0.1 mL 0.55 mmol) and NaBH₃CN (70.24 mg, 1.1 mmol). The mixture was stirred at 60° C. for 4 h. The reaction was monitored by LC/MS. The reaction was quenched with H₂O (30 mL) and extracted with EtOAc (3×30 mL). The combined organic phase was dried over Na₂SO₄, filtered and concentrated. The residue was purified by silica gel column chromatography and using hexane/EtOAc (0-70%) as eluent to afford tert-butyl N-[4-[2-[4-[4-(2,6-dioxo-3-piperidyl)phenyl]piperazin-1-yl]ethyl]cyclohexyl]carbamate (220 mg, 80% yield) as a white solid. MS (ESI) m/z 499.4 [M+H]⁺.

3-[4-[4-[2-(4-Aminocyclohexyl)ethyl]piperazin-1-yl]phenyl]piperidine-2,6-dione. To a stirred solution of tert-butyl N-[4-[2-[4-[4-(2,6-dioxo-3-piperidyl)phenyl]piperazin-1-yl]ethyl]cyclohexyl]carbamate (220 mg, 0.44 mmol) was added HCl in EtOAc (10 mL, 0.38 mmol). The mixture was stirred at rt for 1 h. The reaction was monitored by LC/MS. The solvent was removed in vacuo to afford 3-[4-[4-[2-(4-aminocyclohexyl)ethyl]piperazin-1-yl]phenyl]piperidine-2,6-dione (160 mg, 90.9%) as a yellow solid. MS (ESI) m/z 399.3 [M+H]⁺.

N-[4-[2-[4-[4-(2,6-Dioxo-3-piperidyl)phenyl]piperazin-1-yl]ethyl]cyclohexyl]-3-fluoro-4-[4-(4-oxo-1,5,6,7-tetrahydropyrrolo[3,2-c]pyridin-2-yl)-2-pyridyl]benzamide. To a stirred solution of 3-[4-[4-[2-(4-aminocyclohexyl)ethyl]piperazin-1-yl]phenyl]piperidine-2,6-dione (143 mg, 0.36 mmol) and 3-fluoro-4-[4-(4-oxo-1,5,6,7-tetrahydropyrrolo[3,2-c]pyridin-2-yl)-2-pyridyl]benzoic acid (70 mg, 0.20 mmol) and HATU (151 mg, 0.40 mmol) and DIEA (0.09 mL, 1.13 mmol) was added DMF (4 mL). The mixture was stirred at rt for 2 h. The reaction was monitored by LC/MS. The resulting solution was purified by prep-HPLC (Column: Atlantis T3 OBD, 19×150 mm, 5 mm. Mobile Phase A: Water (0.05% TFA), Mobile Phase B: CH₃CN; Flow rate: 25 mL/min. Detection UV@254/210 nm) to afford N-[4-[2-[4-[4-(2,6-dioxo-3-piperidyl)phenyl]piperazin-1-yl]ethyl]cyclohexyl]-3-fluoro-4-[4-(4-oxo-1,5,6,7-tetrahydropyrrolo[3,2-c]pyridin-2-yl)-2-pyridyl]benzamide (42.9 mg, 27% yield, 0.058 mmol) as a yellow solid. MS (ESI) m/z 732.3 [M+H]^{+ 1}H NMR (400 MHz, DMSO-d₆) δ ppm 12.21 (s, 1H), 10.80 (s, 1H), 9.54 (br s, 1H), 8.68 (d, J=5.6 Hz, 1H), 8.46 (d, J=7.8 Hz, 1H), 8.12 (s, 1H), 8.04-7.95 (m, 1H), 7.90-7.82 (m, 2H), 7.82-7.76 (m, 1H), 7.23 (d, J=2.3 Hz, 1H), 7.17-7.09 (m, 3H), 7.02-6.95 (m, 2H), 3.95-3.72 (m, 4H), 3.61 (d, J=11.7 Hz, 2H), 3.47-3.39 (m, 2H), 3.22 (s, 2H), 3.14 (d, J=10.9 Hz, 2H), 3.02-2.92 (m, 2H), 2.92-2.85 (m, 2H), 2.72-2.59 (m, 1H), 2.49-2.38 (m, 1H), 2.23-2.10 (m, 1H), 2.05-1.96 (m, 1H), 1.91 (d, J=12.0 Hz, 2H), 1.81 (d, J=12.5 Hz, 2H), 1.66-1.59 (m, 2H), 1.48-1.23 (m, 3H), 1.81-1.00 (m, 2H). ¹⁹F NMR (377 MHz, DMSO-d₆) δ ppm −115.8 (s, 1F).

### Example 1.11. 3-(1-Methyl-6-(4-(2-(4-(3-(4-(4-oxo-4,5,6,7-tetrahydro-1H-pyrrolo[3,2-c]pyridin-2-yl)pyridin-2-yl)phenyl)piperazin-1-yl)ethyl)piperazin-1-yl)-1H-indazol-3-yl)piperidine-2,6-dione

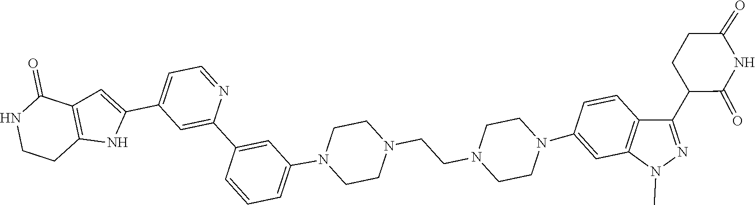 [SMILES: Cn1nc(C2CCC(=O)NC2=O)c2ccc(N3CCN(CCN4CCN(c5cccc(-c6cc(-c7cc8c([nH]7)CCNC8=O)ccn6)c5)CC4)CC3)cc21]

tert-Butyl 4-(3-(4-(4-oxo-4,5,6,7-tetrahydro-1H-pyrrolo[3,2-c]pyridin-2-yl)pyridin-2-yl)phenyl)piperazine-1-carboxylate. 2-(2-chloro-4-pyridyl)-1,5,6,7-tetrahydropyrrolo[3,2-c]pyridin-4-one (100 mg, 0.40 mmol), [3-(4-tert-butoxycarbonylpiperazin-1-yl)phenyl]boronic acid (161 mg, 0.52 mmol), NaHCO₃ (122 mg, 1.21 mmol), Xphos (19.3 mg, 0.04 mmol), 1,4-dioxane (3 mL) and water (1 mL) were added to a microwave tube. The reaction mixture was then sparged with nitrogen for 10 minutes. Xphos Pd G3 (36.9 mg, 0.04 mmol) was then added to the mixture and the reaction mixture was sparged with nitrogen for another 5 minutes. The tube was then sealed and stirred at 90° C. overnight. LCMS showed full conversion. The mixture was then filtered through a Celite pad and rinsed with EtOAc and MeOH. The filtrate was then concentrated and the residue purified by reverse phase flash chromatography (50 g C18 column, liquid deposit (DMSO), elution: 5% MeOH/0.1% HCOOH over 5 CV, then 5 to 100% MeOH/0.1% HCOOH over 20 CV, then 100% MeOH/0.1% HCOOH over 5 CV). Fractions were combined and concentrated to give tert-butyl 4-(3-(4-(4-oxo-4,5,6,7-tetrahydro-1H-pyrrolo[3,2-c]pyridin-2-yl)pyridin-2-yl)phenyl)piperazine-1-carboxylate, bis formic acid salt (202 mg, 64% yield) as a yellow solid, which was used directly in the next step. ¹H NMR (400 MHz, DMSO-d) δ ppm 1.43 (s, 9H), 2.84-2.91 (m, 2H), 3.18-3.22 (m, 4H), 3.43 (br s, 2H), 3.51 (br t, J=5.0 Hz, 4H), 7.03-7.07 (m, 2H), 7.14-7.19 (m, 1H), 7.35-7.40 (m, 1H), 7.55-7.58 (m, 1H), 7.59-7.63 (m, 1H), 7.72 (br s, 1H), 8.15 (s, 3H), 8.54 (d, J=4.9 Hz, 1H), 11.91-12.03 (m, 1H).

2-(2-(3-(Piperazin-1-yl)phenyl)pyridin-4-yl)-1,5,6,7-tetrahydro-4H-pyrrolo[3,2-c]pyridin-4-one. To a solution of tert-butyl 4-[3-[4-(4-oxo-1,5,6,7-tetrahydropyrrolo[3,2-c]pyridin-2-yl)-2-pyridyl]phenyl]piperazine-1-carboxylate, bis formic acid salt (202. mg, 0.26 mmol) in MeOH (4 mL) at room temperature was added 4 M HCl in 1,4-dioxane (0.96 mL, 3.86 mmol). The mixture was stirred at room temperature. After 16 h, LCMS showed full conversion. The solvent was removed under vacuum to give 2-(2-(3-(piperazin-1-yl)phenyl)pyridin-4-yl)-1,5,6,7-tetrahydro-4H-pyrrolo[3,2-c]pyridin-4-one, bis HCl salt (180 mg, quantitative yield) as a yellow solid which was used directly in the next step. ¹H NMR (400 MHz, DMSO-d₆) δ ppm 2.94 (t, J=7.0 Hz, 2H), 3.14-3.21 (m, 1H), 3.23-3.30 (m, 4H), 3.42-3.48 (m, 2H), 3.55-3.62 (m, 4H), 7.25-7.32 (m, 2H), 7.50-7.60 (m, 2H), 7.63-7.74 (m, 2H), 8.06-8.14 (m, 1H), 8.57-8.66 (m, 2H), 9.12-9.22 (m, 2H), 12.93-13.05 (m, 1H).

2-(2-(3-(4-(2,2-Dimethoxyethyl)piperazin-1-yl)phenyl)pyridin-4-yl)-1,5,6,7-tetrahydro-4H-pyrrolo[3,2-c]pyridin-4-one. To a solution of 2-[2-(3-piperazin-1-ylphenyl)-4-pyridyl]-1,5,6,7-tetrahydropyrrolo[3,2-c]pyridin-4-one, dihydrochloride (180 mg, 0.35 mmol) and DIPEA (600 μL, 3.47 mmol) in CH₂Cl₂ (2 mL) and DMSO (2 mL) at rt was added 2,2-dimethoxyacetaldehyde (68 μL, 0.45 mmol) and NaBH(OAc)3 (147 mg, 0.69 mmol) and the mixture was stirred at rt. After 1.5 h, LC/MS showed almost full conversion. The solvent was removed under reduced pressure and the residue was dissolved in DMSO and purified by reverse phase flash chromatography (30 g C18 column, liquid deposit (DMSO+FA), elution: 5% MeOH/0.1% HCOOH over 5 CV, then 5 to 100% MeOH/0.1% HCOOH over 20 CV, then 100% MeOH/0.1% HCOOH over 5 CV). Fractions were combined and concentrated to give 2-(2-(3-(4-(2,2-dimethoxyethyl)piperazin-1-yl)phenyl)pyridin-4-yl)-1,5,6,7-tetrahydro-4H-pyrrolo[3,2-c]pyridin-4-one (159 mg, 99% yield) as a yellow film. ¹H NMR (400 MHz, DMSO-d₆) δ ppm 2.60-2.68 (m, 4H), 2.88 (t, J=6.7 Hz, 2H), 3.20-3.24 (m, 4H), 3.28 (s, 6H), 3.41-3.45 (m, 2H), 4.54 (t, J=5.1 Hz, 1H), 7.00-7.07 (m, 2H), 7.17 (d, J=2.0 Hz, 1H), 7.34 (t, J=7.8 Hz, 1H), 7.54-7.59 (m, 2H), 7.69 (s, 1H), 8.14 (s, 1H), 8.54 (d, J=5.4 Hz, 1H), 11.97 (br s, 1H).

2-[4-[3-[4-(4-Oxo-1,5,6,7-tetrahydropyrrolo[3,2-c]pyridin-2-yl)-2-pyridyl]phenyl]piperazin-1-yl]acetaldehyde. To a round-bottom flask containing 2-[2-[3-[4-(2,2-dimethoxyethyl)piperazin-1-yl]phenyl]-4-pyridyl]-1,5,6,7-tetrahydropyrrolo[3,2-c]pyridin-4-one (159 mg, 0.34 mmol) was added 4 M HCl in 1,4-dioxane (1.3 mL, 5.17 mmol) at rt. The mixture was sonicated for 30 min and stirred at 60° C. for 1.0 h. LCMS showed full conversion. The reaction was cooled down to rt, the solvents were removed under vacuum and excess HCl was removed by co-evaporation with MeCN (3×) to give 2-[4-[3-[4-(4-oxo-1,5,6,7-tetrahydropyrrolo[3,2-c]pyridin-2-yl)-2-pyridyl]phenyl]piperazin-1-yl]acetaldehyde dihydrochloride (180 mg, quantitative yield) as a yellow solid. MS(ESI) m/z: [M+2H]²⁺=208.8.

3-[1-Methyl-6-[4-[2-[4-[3-[4-(4-oxo-1,5,6,7-tetrahydropyrrolo[3,2-c]pyridin-2-yl)-2-pyridyl]phenyl]piperazin-1-yl]ethyl]piperazin-1-yl]indazol-3-yl]piperidine-2,6-dione. To a solution of 2-[4-[3-[4-(4-oxo-1,5,6,7-tetrahydropyrrolo[3,2-c]pyridin-2-yl)-2-pyridyl]phenyl]piperazin-1-yl]acetaldehyde, dihydrochloride (90 mg, 0.18 mmol), 3-(1-methyl-6-piperazin-1-yl-indazol-3-yl)piperidine-2,6-dione, hydrochloride (67 mg, 0.18 mmol) and DIPEA (0.22 mL, 1.29 mmol) in CH₂Cl₂ (2 mL) and DMSO (0.5 mL) at rt was added NaBH(OAc)₃ (78.11 mg, 0.37 mmol). The mixture was then stirred at rt. After 18 h, LC/MS showed full conversion with impurities. The solvent was removed under vacuum and the residue was dissolved in DMSO and purified by reverse phase flash chromatography (50 g C18 column, liquid deposit (DMSO+formic acid), elution: 5% MeCN/0.1% HCOOH over 5 CV, then 5 to 25% MeCN/0.1% HCOOH over 15 CV, then 25 to 100% MeCN/0.1% HCOOH over 2 CV, then 100% MeCN/0.1% HCOOH over 5 CV). Fractions were combined, concentrated and lyophilized to give 3-[1-methyl-6-[4-[2-[4-[3-[4-(4-oxo-1,5,6,7-tetrahydropyrrolo[3,2-c]pyridin-2-yl)-2-pyridyl]phenyl]piperazin-1-yl]ethyl]piperazin-1-yl]indazol-3-yl]piperidine-2,6-dione (14 mg, 9% yield, 0.019 mmol) as a yellow solid as a full formic acid salt. MS(ESI) m/z: [M+H]⁺=727.4. ¹H NMR (400 MHz, DMSO-d6) δ ppm 2.10-2.20 (m, 1H), 2.26-2.35 (m, 1H), 2.54-2.58 (m, 4H), 2.59-2.66 (m, 10H), 2.88 (t, J=6.7 Hz, 2H), 3.21-3.26 (m, 8H), 3.43 (td, J=6.7, 2.0 Hz, 2H), 3.89 (s, 3H), 4.26 (dd, J=9.0, 5.1 Hz, 1H), 6.84 (s, 1H), 6.92 (br d, J=9.8 Hz, 1H), 7.01-7.07 (m, 2H), 7.17 (d, J=2.2 Hz, 1H), 7.34 (t, J=8.1 Hz, 1H), 7.50 (d, J=8.8 Hz, 1H), 7.55-7.59 (m, 2H), 7.70 (s, 1H), 8.14-8.15 (m, 1H), 8.15-8.17 (m, 1H), 8.54 (d, J=5.1 Hz, 1H), 10.85 (s, 1H), 11.97 (br s, 1H).

### Example 1.12. Synthesis of 3-(4-(4-(2-(4-(3-(4-(4-Oxo-4,5,6,7-tetrahydro-1H-pyrrolo[3,2-c]pyridin-2-yl)pyridin-2-yl)phenyl)piperazin-1-yl)ethyl)piperazin-1-yl)phenyl)piperidine-2,6-dione

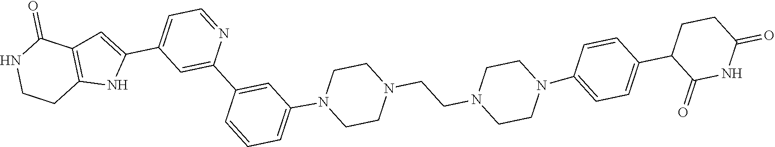 [SMILES: O=C1CCC(c2ccc(N3CCN(CCN4CCN(c5cccc(-c6cc(-c7cc8c([nH]7)CCNC8=O)ccn6)c5)CC4)CC3)cc2)C(=O)N1]

3-(4-(4-(2-(4-(3-(4-(4-Oxo-4,5,6,7-tetrahydro-1H-pyrrolo[3,2-c]pyridin-2-yl)pyridin-2-yl)phenyl)piperazin-1-yl)ethyl)piperazin-1-yl)phenyl)piperidine-2,6-dione. To a solution of 2-[4-[3-[4-(4-oxo-1,5,6,7-tetrahydropyrrolo[3,2-c]pyridin-2-yl)-2-pyridyl]phenyl]piperazin-1-yl]acetaldehyde; dihydrochloride (90.0 mg, 0.18 mmol) and 3-(4-piperazin-1-ylphenyl)piperidine-2,6-dione; dihydrochloride (70.2 mg, 0.20 mmol) in DCM (1.5 mL) and DMSO (0.5 mL) was added DIPEA (0.26 mL, 1.47 mmol) and the solution was stirred for 10 min at rt. NaBH(OAc)₃ (78 mg, 0.37 mmol) was then added and the reaction mixture was stirred at rt for 3 h. The DCM was evaporated and the reaction mixture was directly loaded on a C18 50 g column and purified by prep-HPLC (Isco, MeCN/H₂O+0.1% FA, 4 CV 5% MeCN/H₂O+0.1% FA, then 2 CV 5 to 30% MeCN and the 15 CV 30 to 100% MeCN, to give, after lyophilization, 3-(4-(4-(2-(4-(3-(4-(4-oxo-4,5,6,7-tetrahydro-1H-pyrrolo[3,2-c]pyridin-2-yl)pyridin-2-yl)phenyl)piperazin-1-yl)ethyl)piperazin-1-yl)phenyl)piperidine-2,6-dione as a partial formic acid salt (1:0.52) (12 mg, 99.9% purity at 215 nm) as a yellow solid. MS(ESI) m/z: [M+H]⁺=673.4. ¹H NMR (400 MHz, DMSO-d6) δ ppm 1.96-2.05 (m, 1H), 2.07-2.20 (m, 1H), 2.52-2.66 (m, 13H), 2.88 (t, J=6.8 Hz, 2H), 3.12 (br s, 4H), 3.23 (br s, 4H), 3.43 (td, J=6.9, 2.1 Hz, 3H), 3.72 (dd, J=11.2, 4.9 Hz, 1H), 6.89 (d, J=8.6 Hz, 2H), 6.99-7.08 (m, 4H), 7.17 (d, J=2.2 Hz, 1H), 7.34 (t, J=8.1 Hz, 1H), 7.54-7.61 (m, 2H), 7.70 (s, 1H), 8.15 (br s, 1H), 8.54 (d, J=5.1 Hz, 1H), 10.77 (s, 1H), 11.97 (br s, 1H).

### Example 1.13. Synthesis of 3-(7-(4-(2-(4-(3-Fluoro-4-(4-(4-oxo-4,5,6,7-tetrahydro-1H-pyrrolo[3,2-c]pyridin-2-yl)pyridin-2-yl)phenyl)piperazin-1-yl)ethyl)piperazin-1-yl)-1-methyl-1H-indazol-3-yl)piperidine-2,6-dione

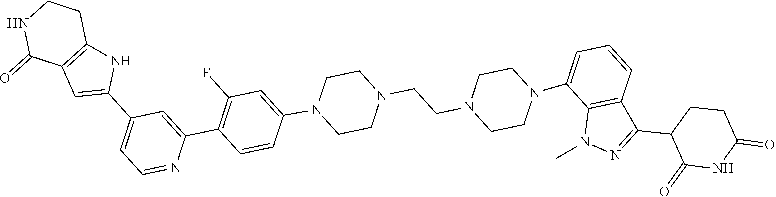 [SMILES: Cn1nc(C2CCC(=O)NC2=O)c2cccc(N3CCN(CCN4CCN(c5ccc(-c6cc(-c7cc8c([nH]7)CCNC8=O)ccn6)c(F)c5)CC4)CC3)c21]

tert-Butyl 4-(3-fluoro-4-(4-(4-oxo-4,5,6,7-tetrahydro-1H-pyrrolo[3,2-c]pyridin-2-yl)pyridin-2-yl)phenyl)piperazine-1-carboxylate. To a microwave tube was added 2-(2-chloro-4-pyridyl)-1,5,6,7-tetrahydropyrrolo[3,2-c]pyridin-4-one (300 mg, 1.21 mmol) tert-butyl 4-[3-fluoro-4-(4,4,5,5-tetramethyl-1,3,2-dioxaborolan-2-yl)phenyl]piperazine-1-carboxylate (640 mg, 1.57 mmol) and Cs₂CO₃ (1.18 g, 3.63 mmol) in 1,4-dioxane (4.5 mL) and water (1.5 mL). The mixture was sparged with nitrogen. After 10 min, Pd(dppf)Cl₂·CH₂Cl₂ (148 mg, 0.18 mmol) was added and the mixture was sparged with nitrogen. After 5 min, the tube was sealed and it was stirred at 90° C. After 18 h, LCMS showed full conversion. The reaction was filtered over a pad of Celite, rinsed with EtOAc and MeOH and concentrated to dryness. The residue was purified by reverse phase flash chromatography (100 g C18 column, liquid deposit (DMSO), elution: 5% MeOH/0.1% HCOOH over 5 CV, then 5 to 70% MeOH/0.1% HCOOH over 15 CV, then 70 to 100% MeOH/0.1% HCOOH over 10 CV). Fractions were combined and concentrated to give tert-butyl 4-(3-fluoro-4-(4-(4-oxo-4,5,6,7-tetrahydro-1H-pyrrolo[3,2-c]pyridin-2-yl)pyridin-2-yl)phenyl)piperazine-1-carboxylate, formic acid salt (550 mg, 84% yield) as a yellow solid. MS(ESI) m/z: [M+H]⁺=492.2. ¹H NMR (400 MHz, DMSO-d₆) δ ppm 1.43 (s, 9H), 2.85 (t, J=6.8 Hz, 2H), 3.24-3.28 (m, 4H), 3.38-3.44 (m, 2H), 3.46 (br d, J=4.9 Hz, 4H), 6.84-6.93 (m, 2H), 6.98 (d, J=2.2 Hz, 1H), 7.06 (br s, 1H), 7.51 (dd, J=5.3, 1.3 Hz, 1H), 7.80-7.87 (m, 1H), 7.89 (s, 1H), 8.14 (s, 1H), 8.53 (d, J=5.1 Hz, 1H), 11.98 (br s, 1H). ¹⁹F NMR (377 MHz, DMSO-d₆) δ ppm −114.65 (dd, J=15.0, 9.5 Hz, 1F).

2-(2-(2-Fluoro-4-(piperazin-1-yl)phenyl)pyridin-4-yl)-1,5,6,7-tetrahydro-4H-pyrrolo[3,2-c]pyridin-4-one. To a round-bottom flask was added tert-butyl 4-[3-fluoro-4-[4-(4-oxo-1,5,6,7-tetrahydropyrrolo[3,2-c]pyridin-2-yl)-2-pyridyl]phenyl]piperazine-1-carboxylate; formic acid (744 mg, 1.38 mmol), MeOH (5 mL) and 4 M HCl in 1,4-dioxane (3.78 mL, 15.14 mmol). The reaction mixture was stirred at room temperature. After 5 h, HPLC showed full conversion. Volatiles were removed in vacuo and the residue was chased-off with MeCN, dried in the high-vacuum pump to give 2-(2-(2-fluoro-4-(piperazin-1-yl)phenyl)pyridin-4-yl)-1,5,6,7-tetrahydro-4H-pyrrolo[3,2-c]pyridin-4-one, hydrochloric acid salt (617 mg, quantitative yield) as a yellow solid as a. MS(ESI) m/z: [M+H]⁺=392.2.

2-(2-(4-(4-(2,2-Dimethoxyethyl)piperazin-1-yl)-2-fluorophenyl)pyridin-4-yl)-1,5,6,7-tetrahydro-4H-pyrrolo[3,2-c]pyridin-4-one. To a solution of 2-[2-(2-fluoro-4-piperazin-1-yl-phenyl)-4-pyridyl]-1,5,6,7-tetrahydropyrrolo[3,2-c]pyridin-4-one; hydrochloride (617 mg, 1.38 mmol) in DCM (16.4 mL) was added 2,2-dimethoxyacetaldehyde 60% w/w in H₂O (0.49 mL, 3.27 mmol), DIPEA (0.85 mL, 4.91 mmol) and NaBH(OAc)3 (693 mg, 3.27 mmol). The reaction was stirred at rt. After 18 h, LCMS showed incomplete conversion. Additional NaBH(OAc)₃ (693 mg, 3.27 mmol), DIPEA (1.70 mL, 9.82 mmol) and 2,2-dimethoxyacetaldehyde 60% w/w in H₂O (0.49 mL, 3.27 mmol) were added and the reaction was stirred at rt. After 2 h, HPLC still showed incomplete conversion. The solvent was removed under vacuum and the residue was taken up with DMSO (15 mL). NaBH(OAc)₃ (693 mg, 3.27 mmol, 2.4 equiv), 2,2-dimethoxyacetaldehyde 60% w/w in H₂O (0.49 mL, 3.27 mmol) and DIPEA (0.85 mL, 4.91 mmol) were added and the reaction mixture was stirred at 60° C. After 2 h, LCMS showed the reaction was completed. The reaction was directly purified by reverse phase flash chromatography (150 g C18 column, liquid deposit (DMSO), elution: 5% MeOH/0.1% HCOOH over 3 CV, then 5 to 70% MeOH/0.1% HCOOH over 15 CV). Fractions were combined and concentrated to give 2-(2-(4-(4-(2,2-dimethoxyethyl)piperazin-1-yl)-2-fluorophenyl)pyridin-4-yl)-1,5,6,7-tetrahydro-4H-pyrrolo[3,2-c]pyridin-4-one (585 mg, 82% yield) as a yellow solid. MS(ESI) m/z: [M+H]⁺=480.2. ¹H NMR (400 MHz, DMSO-d₆) δ ppm 2.49-2.52 (m, 2H), 2.58-2.63 (m, 4H), 2.85 (t, J=6.8 Hz, 2H), 3.24-3.30 (m, 10H), 3.42 (td, J=6.8, 2.1 Hz, 2H), 4.54 (t, J=5.1 Hz, 1H), 6.81-6.91 (m, 2H), 6.98 (d, J=2.2 Hz, 1H), 7.06 (br s, 1H), 7.51 (dd, J=5.4, 1.5 Hz, 1H), 7.83 (t, J=9.2 Hz, 1H), 7.89 (s, 1H), 8.54 (d, J=5.4 Hz, 1H), 11.98 (br s, 1H). ¹⁹F NMR (377 MHz, DMSO-d₆) δ −114.73 (dd, J=15.0, 9.5 Hz, 1F).

2-(4-(3-Fluoro-4-(4-(4-oxo-4,5,6,7-tetrahydro-1H-pyrrolo[3,2-c]pyridin-2-yl)pyridin-2-yl)phenyl)piperazin-1-yl)acetaldehyde. To a solution of 2-[2-[4-[4-(2,2-dimethoxyethyl)piperazin-1-yl]-2-fluoro-phenyl]-4-pyridyl]-1,5,6,7-tetrahydropyrrolo[3,2-c]pyridin-4-one (140 mg, 0.29 mmol) in 1,4-dioxane (1 mL) was added 4 M HCl in 1,4-dioxane (1.1 mL, 4.38 mmol) and few drops of water. The reaction was stirred at room temperature. After 22 h, LCMS showed full conversion. Volatiles were removed under vacuum to give 2-(4-(3-fluoro-4-(4-(4-oxo-4,5,6,7-tetrahydro-1H-pyrrolo[3,2-c]pyridin-2-yl)pyridin-2-yl)phenyl)piperazin-1-yl)acetaldehyde, bis hydrochloric acid salt (147 mg, quantitative yield) as a yellow solid. MS(ESI) m/z: [M+2H]²⁺=217.8.

3-(7-(4-(2-(4-(3-Fluoro-4-(4-(4-oxo-4,5,6,7-tetrahydro-1H-pyrrolo[3,2-c]pyridin-2-yl)pyridin-2-yl)phenyl)piperazin-1-yl)ethyl)piperazin-1-yl)-1-methyl-1H-indazol-3-yl)piperidine-2,6-dione. To a suspension of 2-[4-[3-fluoro-4-[4-(4-oxo-1,5,6,7-tetrahydropyrrolo[3,2-c]pyridin-2-yl)-2-pyridyl]phenyl]piperazin-1-yl]acetaldehyde; dihydrochloride (147 mg, 0.29 mmol), 3-(1-methyl-7-piperazin-1-yl-indazol-3-yl)piperidine-2,6-dione; dihydrochloride (95 mg, 0.24 mmol) and DIPEA (0.21 mL, 1.19 mmol, 5 equiv) in DCE (2.5 mL) was added NaBH(OAc)₃ (126 mg, 0.59 mmol, 2.5 equiv). The reaction was stirred at room temperature. After 16 h, LCMS showed incomplete conversion. An additional portion of NaBH(OAc)₃ (80 mg, 0.38 mmol) was added followed by the addition of DMSO (1 mL). The reaction was stirred at rt for 24 h, LCMS indicated that the reaction was still not complete. Finally, after the addition of a third portion of NaBH(OAc)₃ (85 mg, 0.40 mmol) and heating the reaction at 50° C. for 5.5 h, LC/MS indicated that the starting material has been fully converted. The solvent was removed under vacuum and the residue was purified by reverse phase flash chromatography (50 g C18 column, liquid deposit (DMSO), elution: 5% MeCN/0.1% HCOOH over 3 CV, then 5 to 20% MeCN/0.1% HCOOH over 17 CV, then 20% MeCN/0.1% HCOOH over 5 CV). Fractions were combined, concentrated and lyophilized to give 3-(7-(4-(2-(4-(3-Fluoro-4-(4-(4-oxo-4,5,6,7-tetrahydro-1H-pyrrolo[3,2-c]pyridin-2-yl)pyridin-2-yl)phenyl)piperazin-1-yl)ethyl)piperazin-I-yl)-1-methyl-1H-indazol-3-yl)piperidine-2,6-dione, formic acid salt (35.4 mg, 60% yield, 0.047 mmol) as a yellow solid. MS(ESI) m/z: [M+H]⁺=745.4. ¹H NMR (400 MHz, DMSO-d₆) δ ppm 2.12-2.21 (m, 1H), 2.26-2.41 (m, 2H), 2.52-2.70 (m, 10H), 2.78-3.22 (m, 7H), 3.26-3.30 (m, 4H), 3.39-3.43 (m, 2H), 4.25 (s, 3H), 4.34 (dd, J=9.7, 5.0 Hz, 1H), 6.82-6.92 (m, 2H), 6.98 (d, J=2.2 Hz, 1H), 6.99-7.07 (m, 3H), 7.39 (dd, J=7.2, 1.6 Hz, 1H), 7.51 (dd, J=5.3, 1.3 Hz, 1H), 7.83 (t, J=9.2 Hz, 1H), 7.89 (s, 1H), 8.15 (s, 1H), 8.53 (d, J=5.4 Hz, 1H), 10.88 (s, 1H), 11.98 (br s, 1H). ¹⁹F NMR (377 MHz, DMSO-d₆) δ −114.64-−114.57 (m, 1F).

### Example 2. Biological Assays

### Example 2.1. MK2 Degradation Assay in MK2 Overexpressed HiBiT-Tagged Cell Line

A compound was dispensed into a white 384-well tissue-culture treated plate using an acoustic liquid handler. Dilutions were based on a 25 μL assay volume in duplicate 10 point 3-fold serial dilutions starting with a 10 μM dose. Negative control wells containing only 0.2% DMSO were also included to calculate 100% signal. All wells were backfilled to a final DMSO concentration of 0.2% to ensure DMSO uniformity across wells. Cells stably expressing HiBiT-tagged protein of interest were then washed, trypsinized, and counted.

These cells were then resuspended in fresh medium to give the proper concentration. This was done so that when cells were plated at a 25 μL seeding volume the assay would be conducted within the linear range from optimization of cell-based HiBiT assay. Following the above procedure, 25 μL of cells expressing HiBiT-tagged protein of interest were dispensed per well. Cells were then seeded into the 384-well plate that was pre-spotted with compounds. The resulting cells were incubated overnight at 37° C./5% CO₂.

The 384-well plates were then removed from the incubator and left at room temperature for 30 min. The Nano-Glo HiBiT Lytic Detection Reagent was prepared according to manufacturer's instructions. 25 μL of Nano-Glo HiBiT Lytic Detection Reagent were added per well of a 384-well plate. The resulting plates were incubates for 30 min at room temperature. Luminescence signal was then read using a plate reader.

All luminescence values were normalized to the DMSO control wells. The average value of the DMSO control wells was set to equal 100% of the relative HiBiT-tagged target protein levels.

The luminescence values were plotted using a graphing software. Compound concentration was plotted on the x-axis and the corresponding relative protein of interest levels on the y-axis. Graphing software was used to determine the EC₅₀ value (the half-maximum effective concentration) of a compound for the degradation of the HiBiT-tagged substrate. The software used a four-parameter logistic model (sigmoidal dose-response model) (FIT=(A+{(B−A)/1+[(C/x)^{D}]})) where C was the inflection point (EC₅₀), D was the correlation coefficient, and A and B were the low and high limits of the fit, respectively) to calculate the EC₅₀. The Yₘᵢₙ was calculated by determining the lowest percentage of target protein remaining following compound treatment.

### Example 2.2. Mitogen-Activated Protein Kinase-Activated Protein Kinase 2 AssayQuant Technologies® Assay for Compound Potency Assessment (MK2 OMNIA Biochemical Assay)

The protocol below describes a continuous-read kinase assay optimized to measure potency of compounds against p38a activated, mitogen-activated protein kinase-activated protein kinase 2 (MAPKAP-K2 or MK2) enzyme.

[Reagent] used: [MK-2]=0.05 nM, [ATP]=100 μM and [AQT0425]=10 μM

A 1.25× stock solution of MK2 (PV3317, from Invitrogen) and a 5× stock solution of ATP and Sox conjugated peptide substrate, AQT0425 (CSKS-AQT0425B, from AssayQuant Technologies), were prepared in 1× kinase reaction buffer consisting of 50 mM HEPES, pH 7.5, 0.01% Brij-35, 0.55 mM EGTA, 10 mM MgCl2 and 1 mM DTT.

10 μL of the ATP and substrate solution mix was added to a Corning (3574) 384-well, white, non-binding surface microtiter plate containing 0.5 L of serially diluted test compounds prepared in DMSO. The reactions were started with the addition of 40 μL of the enzyme solution and monitored every 71 seconds for 240 min at λex 360/λem 485 in a Synergy H4 plate reader from BioTek at room temperature.

The initial linear portions of the net progress curves were fit according to a linear equation to yield the slope and percentage of inhibition (% inhibition) at each compound concentration. The net progress curves obtained during the entirety of reactions were also fit according to an ascending single-exponential equation (Eq. 1) to yield V_{obs} values at each compound concentration. Plots of % Inhibition versus degrader concentrations were fit according to a dose-response equation (Eq. 2) to generate IC₅₀ and Hill slope values while plots of V_{obs} versus degrader concentration were fit according to Equation 3 (Eq. 3) to generate apparent K_{inact} values using the GraphPad PRISM software.

$$V = \text{?} \qquad ( Eq . 1 )$$

$$\text{?} \text{indicates text missing or illegible when filed}$$

where F is the fluorescence intensity from the plate reader, V₀ is a constant reflecting the relationship between the instrument readout and product concentration, t is time, e is Euler's number, and V_{obs} is the observed inactivation rate constant.

$$\% Inhibition = \frac{100}{\text{?}} \qquad ( Eq . 2 )$$

$$\text{?} \text{indicates text missing or illegible when filed}$$

where % Inhibition is percentage of inhibition, IC₅₀ is half maximal inhibitory concentration, [I] is the degraders concentration, and n is the Hill slope.

$${k}_{obs} = \text{?} \qquad ( Eq . 3 )$$

$$\text{?} \text{indicates text missing or illegible when filed}$$

where k_{obs} is the observed inactivation rate constant, K_{inact} is the apparent inactivation rate constant, K_{I} is the apparent inhibition constant, and [I] is the degrader concentration.

Using these assays, the EC₅₀ and IC₅₀ values of the following compounds were determined. See Table 2. Compounds having an EC₅₀ of <10 nM in the HiBiT assay are denoted A; compounds having an EC₅₀ of 10-100 nM in the HiBiT assay are denoted B; compounds having an EC₅₀ of 101-500 nM in the HiBiT assay are denoted C; compounds having an EC₅₀ of >500 nM in the HiBiT assay are denoted D; compounds having an IC₅₀ of <150 nM in the OMNIA assay are denoted E; compounds having an IC₅₀ of 151-500 nM in the OMNIA assay are denoted F; compounds having an IC₅₀ of 501-1000 nM in the OMNIA assay are denoted G.

TABLE 2

| | | MK2 100 μM ATP OMNIA |
|---|---|---|
| Example | HiBiT MK2 OE EC₅₀ (nM) | IC₅₀ (nM) |
| 1.5 | A | E |
| 1.6 | B | F |
| 1.7 | B | F |
| 1.8 | A | G |
| 1.9 | C | F |
| 1.10 | B | G |
| 1.11 | C | E |
| 1.12 | D | F |
| 1.13 | A | G |

### EMBODIMENTS

Embodiment 1: A compound of Formula I:

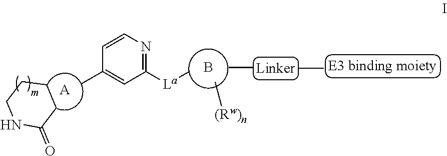

or a pharmaceutically acceptable salt thereof, wherein:
the Linker is a bivalent group;
the E3 binding moiety is a moiety that binds to an E3 ubiquitin ligase protein;
Ring A is selected from

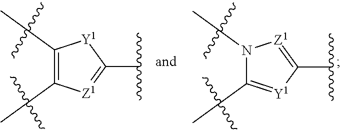

Y¹ is selected from —O—, —S—, and —N(R)—;
each of Y² and Z¹ is selected from —C(R¹)— and —N—;
R¹ is selected from hydrogen, halogen, and optionally substituted C₁₋₆ aliphatic;
Ring B is a phenylene, a 5- to 6-membered heteroarylene ring having 1-4 heteroatoms independently selected from nitrogen, oxygen, and sulfur, or a 3- to 7-membered saturated or partially unsaturated heterocyclylene having 1-3 heteroatoms independently selected from nitrogen, oxygen, and sulfur;
L^{a} is selected from a covalent bond, —O—, —S—, —N(R)—, and C₁₋₆ aliphatic;
R^{w} is selected from halogen, —OR, —SR, —CN, —NO₂, —SO₂NR, —SO₂R, —SOR, —C(O)R, —CO₂R, —C(O)N(R)₂, —NRC(O)R, —NRC(O)OR, —NRC(O)N(R)₂, —NRSO₂R, —N(R)₂, or an optionally substituted group selected from the group consisting of C₁₋₆ aliphatic, phenyl, a 3- to 8-membered saturated or partially unsaturated carbocyclic ring, a 4- to 7-membered heterocyclic ring having 1-2 heteroatoms independently selected from nitrogen, oxygen, or sulfur, and a 5- to 6-membered monocyclic heteroaryl ring having 1-4 heteroatoms independently selected from nitrogen, oxygen, or sulfur,
each R is independently at each occurrence hydrogen or optionally substituted C₁₋₆ aliphatic; and
each of m and n is 0, 1, 2, or 3.

Embodiment 2: The compound according to Embodiment 1, wherein Ring A is

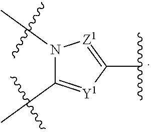

Embodiment 3: The compound according to Embodiment 1, wherein the compound is a compound of Formula I-a:

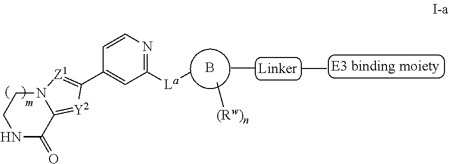

or a pharmaceutically acceptable salt thereof.

Embodiment 4: The compound according to Embodiment 3, wherein Y² is —C(R¹)—.

Embodiment 5: The compound according to Embodiment 3 or Embodiment 4, wherein Z¹ is —C(R¹)—.

Embodiment 6: The compound according to Embodiment 4 or Embodiment 5, wherein R¹ is hydrogen.

Embodiment 7: The compound according to Embodiment 1, wherein Ring A is

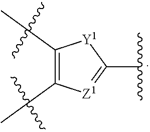

Embodiment 8: The compound according to Embodiment 1, wherein the compound is a compound of Formula I-b:

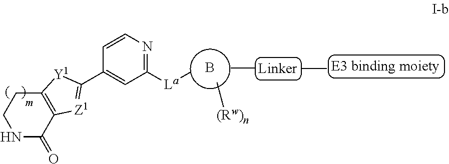

or a pharmaceutically acceptable salt thereof.

Embodiment 9: The compound according to Embodiment 8, wherein Y¹ is —N(R)—.

Embodiment 10: The compound according to Embodiment 9, wherein R is hydrogen.

Embodiment 11: The compound according to any one of Embodiments 8-10, wherein Z¹ is —C(R¹)—.

Embodiment 12: The compound according to Embodiment 11, wherein R¹ is hydrogen.

Embodiment 13: The compound according to any one of Embodiments 1-12, wherein Ring B is phenylene.

Embodiment 14: The compound according to any one of Embodiments 1-13, wherein L^{a} is a covalent bond.

Embodiment 15: The compound according to any one of Embodiments 1-14, wherein R^{w} is halogen.

Embodiment 16: The compound according to any one of Embodiments 1-15, wherein m is 1.

Embodiment 17: The compound according to any one of Embodiments 1-16, wherein n is 0.

Embodiment 18: The compound according to any one of Embodiments 1-16, wherein n is 1.

Embodiment 19: The compound according to Embodiment 1, wherein the compound is a compound of Formulae I-a-i, I-a-ii, I-a-iii, I-a-iv, I-b-i, I-b-ii, I-b-iii, and I-b-iv:

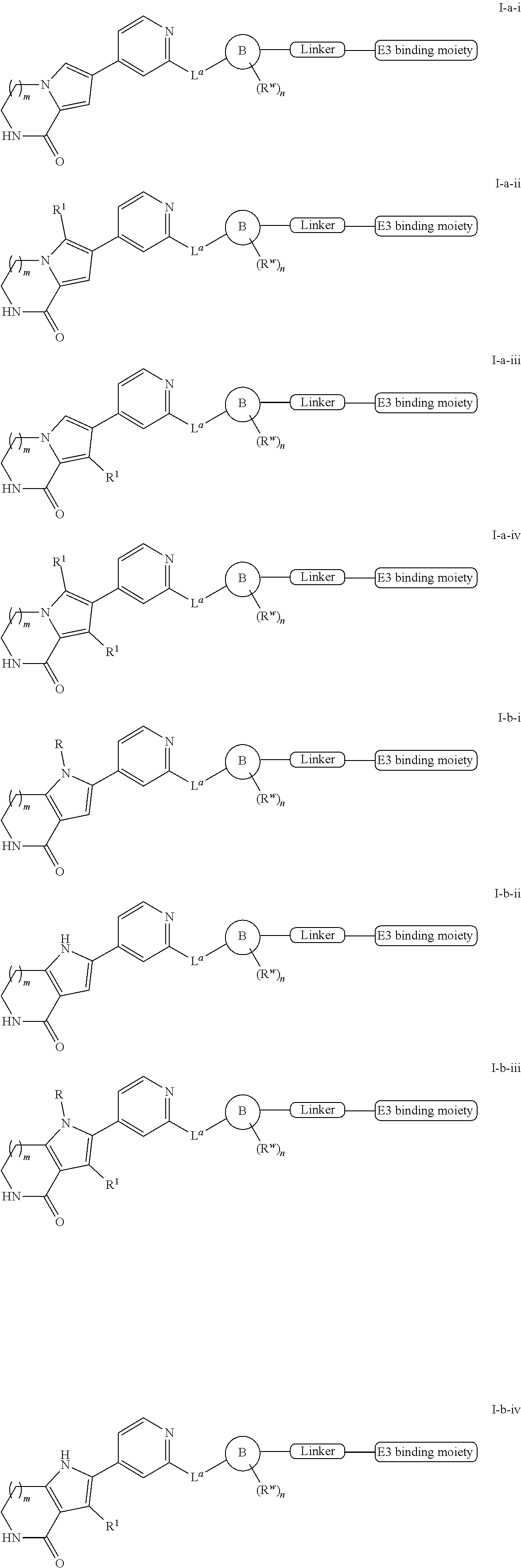

or a pharmaceutically acceptable salt thereof.

Embodiment 20: The compound according to any one of Embodiments 1-19, wherein:
the Linker is an optionally substituted bivalent C₂₋₂₀ straight or branched aliphatic chain, wherein one, two, three, four, or five methylene units of the aliphatic chain are optionally and independently replaced by a group selected from —N(R)—, —C(O)—, —C(O)N(R)—, a 4- to 6-membered monocyclic saturated ring having 0-2 heteroatoms independently selected from nitrogen, a bivalent 6- to 8-membered saturated or partially unsaturated bridged bicyclic, or a fused bicyclic or spirofused heterocyclic ring having 1-3 heteroatoms independently selected from nitrogen, oxygen, and sulfur;
the Linker is optionally substituted with 0, 1, or 2 instances of R^{L}; and
R^{L} is independently selected from —OR, or an optionally substituted group selected from the group consisting of C₁₋₆ aliphatic chain.

Embodiment 21: The compound according to Embodiment 20, wherein:
the Linker is an optionally substituted bivalent C₂₋₂₀ straight or branched aliphatic chain, wherein two, three, or four methylene units of the aliphatic chain are optionally and independently replaced by a group selected from —N(R)—, —O—, —C(O)—, —N(R)C(O)—, —C(O)N(R)—, —and a bivalent 4- to 6-membered monocyclic saturated ring having 0-2 heteroatoms independently selected from nitrogen; and
the bivalent 4- to 6-membered monocyclic saturated ring is substituted by 0-4 instances of R^{L}.

Embodiment 22: The compound according to Embodiment 20, wherein the L Linker is an optionally substituted bivalent C₂₋₂₀ straight or branched aliphatic chain, wherein two, three, four, or five methylene units of the bivalent C₂₋₂₀ straight or branched aliphatic chain are optionally and independently replaced by a group selected from —C(O)N(R)—, —N(R)—, —C(O)—,

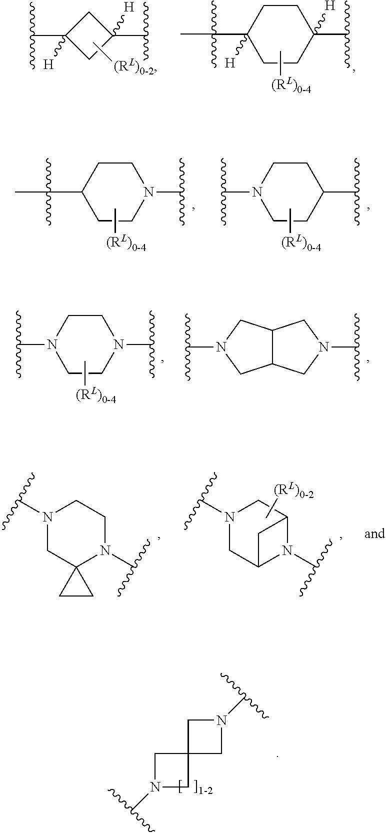

Embodiment 23: The compound according to Embodiment 20, wherein the Linker is an optionally substituted bivalent C₂₋₂₀ straight or branched aliphatic chain, wherein two, three, four, or five methylene units of the aliphatic chain are optionally and independently replaced by a group selected from —C(O)N(R)—, —N(R)—, —C(O),

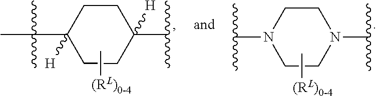

Embodiment 24: The compound according to Embodiment 20, wherein the Linker is selected from the group consisting of:

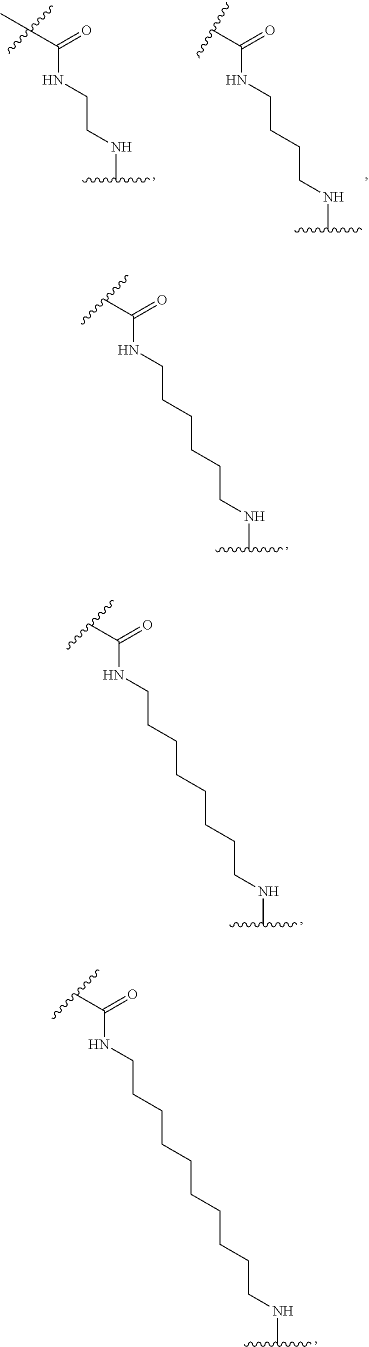 [SMILES: *NCCNC(*)=O ; *NCCCCNC(*)=O ; *NCCCCCCNC(*)=O ; *NCCCCCCCCNC(*)=O ; *NCCCCCCCCCCNC(*)=O]

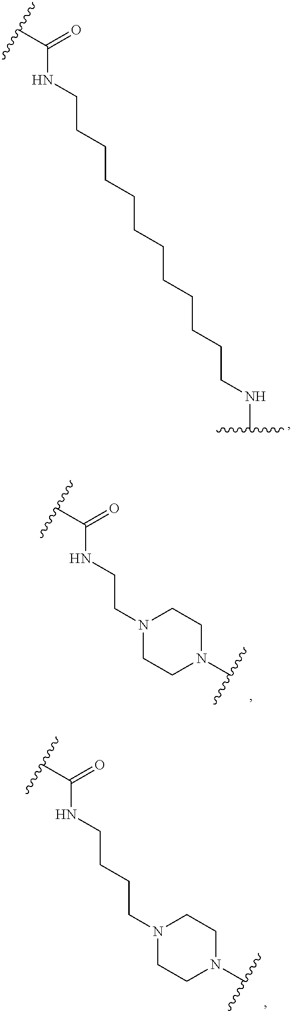 [SMILES: *NCCCCCCCCCCCCNC(*)=O ; *C(=O)NCCN1CCN(*)CC1 ; *C(=O)NCCCCN1CCN(*)CC1]

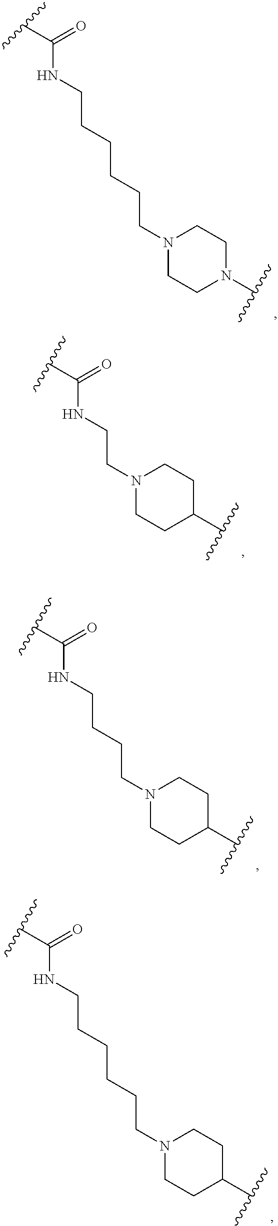 [SMILES: *C(=O)NCCCCCCN1CCN(*)CC1 ; *C(=O)NCCN1CCC(*)CC1 ; *C(=O)NCCCCN1CCC(*)CC1 ; *C(=O)NCCCCCCN1CCC(*)CC1]

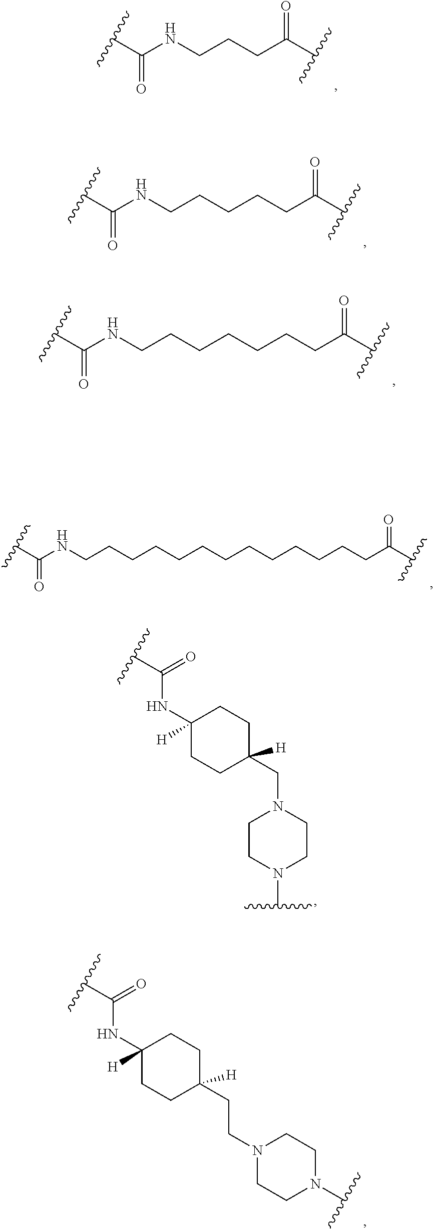 [SMILES: *C(=O)CCCNC(*)=O ; *C(=O)CCCCCNC(*)=O ; *C(=O)CCCCCCCNC(*)=O ; *C(=O)CCCCCCCCCCCCCNC(*)=O ; *C(=O)N[C@H]1CC[C@H](CN2CCN(*)CC2)CC1 ; *C(=O)N[C@H]1CC[C@H](CCN2CCN(*)CC2)CC1]

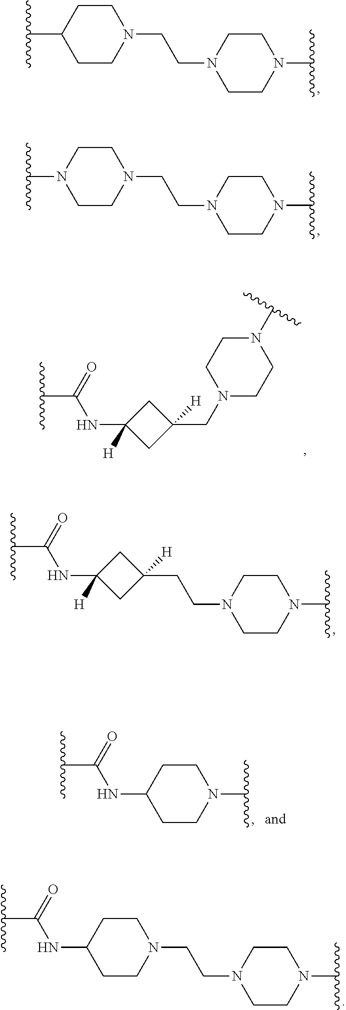 [SMILES: *C1CCN(CCN2CCN(*)CC2)CC1 ; *N1CCN(CCN2CCN(*)CC2)CC1 ; *C(=O)N[C@H]1C[C@H](CN2CCN(*)CC2)C1 ; *C(=O)N[C@H]1C[C@H](CCN2CCN(*)CC2)C1 ; *C(=O)NC1CCN(*)CC1 ; *C(=O)NC1CCN(CCN2CCN(*)CC2)CC1]

Embodiment 25: The compound according to Embodiment 20, wherein the Linker is selected from the group consisting of:

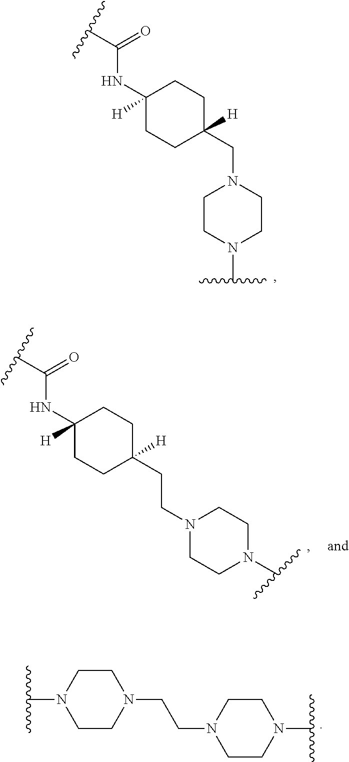 [SMILES: *C(=O)N[C@H]1CC[C@H](CN2CCN(*)CC2)CC1 ; *C(=O)N[C@H]1CC[C@H](CCN2CCN(*)CC2)CC1 ; *N1CCN(CCN2CCN(*)CC2)CC1]

Embodiment 26: The compound according to any one of Embodiments 1-25, wherein the E3 binding moiety is a cereblon protein binding moiety.

Embodiment 27: The compound according to Embodiment 26, wherein the cereblon protein binding moiety is selected from the group consisting of:

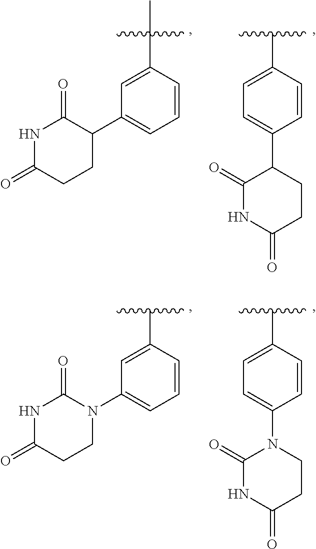

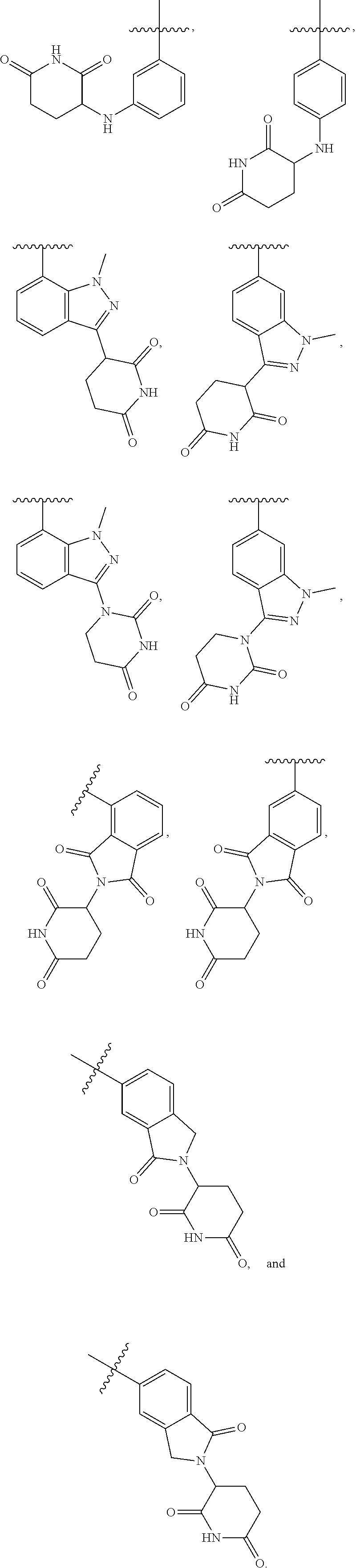

Embodiment 27: The compound according to Embodiment 26, wherein the cereblon protein binding moiety is selected from the group consisting of:

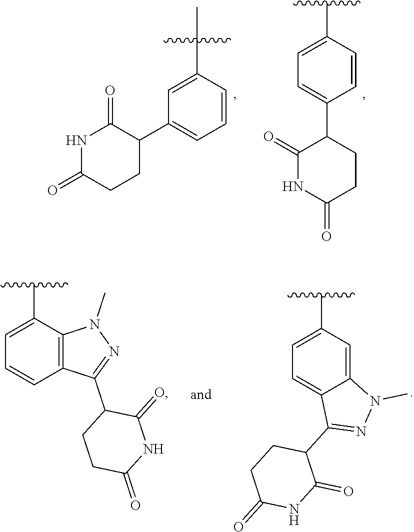

Embodiment 29: The compound according to Embodiment 1, wherein the compound is selected from Table 1:

| | |
|---|---|
| 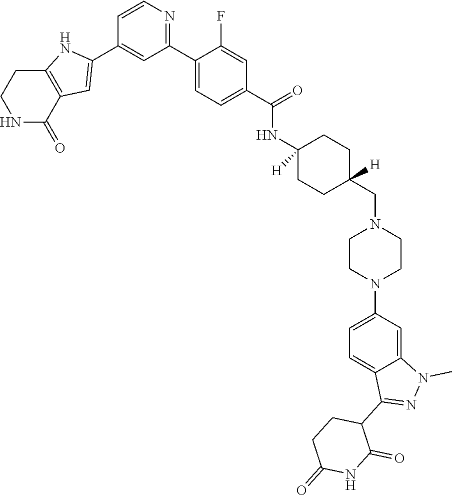 [SMILES: Cn1nc(C2CCC(=O)NC2=O)c2ccc(N3CCN(C[C@H]4CC[C@H](NC(=O)c5ccc(-c6cc(-c7cc8c([nH]7)CCNC8=O)ccn6)c(F)c5)CC4)CC3)cc21] | Ex. 1.5 |
| 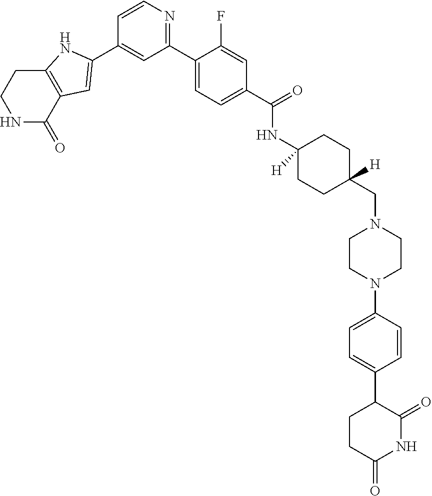 [SMILES: O=C1CCC(c2ccc(N3CCN(C[C@H]4CC[C@H](NC(=O)c5ccc(-c6cc(-c7cc8c([nH]7)CCNC8=O)ccn6)c(F)c5)CC4)CC3)cc2)C(=O)N1] | Ex 1.6 |
| 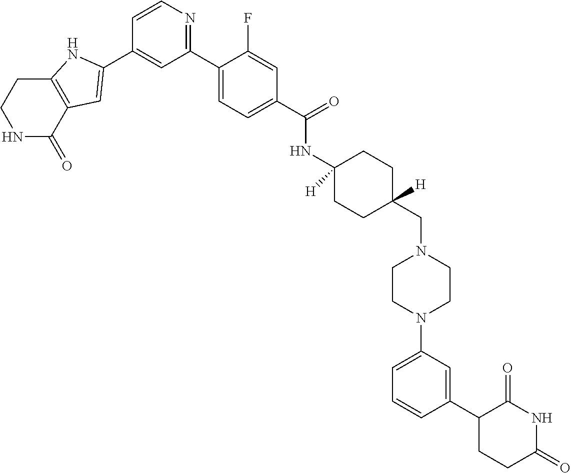 [SMILES: O=C1CCC(c2cccc(N3CCN(C[C@H]4CC[C@H](NC(=O)c5ccc(-c6cc(-c7cc8c([nH]7)CCNC8=O)ccn6)c(F)c5)CC4)CC3)c2)C(=O)N1] | Ex 1.7 |
| 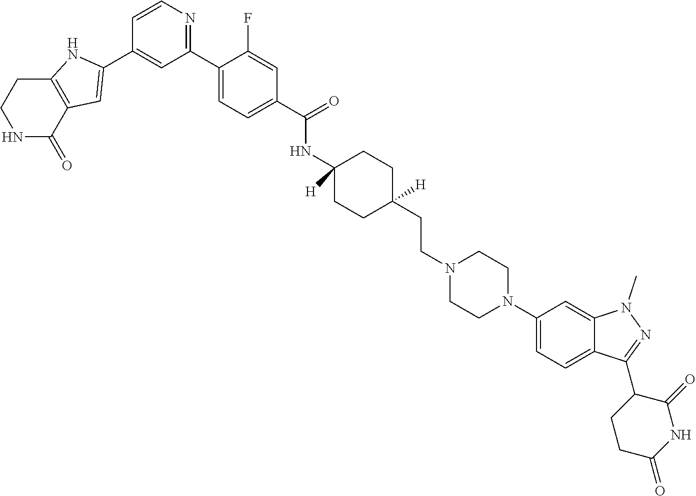 [SMILES: Cn1nc(C2CCC(=O)NC2=O)c2ccc(N3CCN(CC[C@H]4CC[C@H](NC(=O)c5ccc(-c6cc(-c7cc8c([nH]7)CCNC8=O)ccn6)c(F)c5)CC4)CC3)cc21] | Ex 1.8 |
| 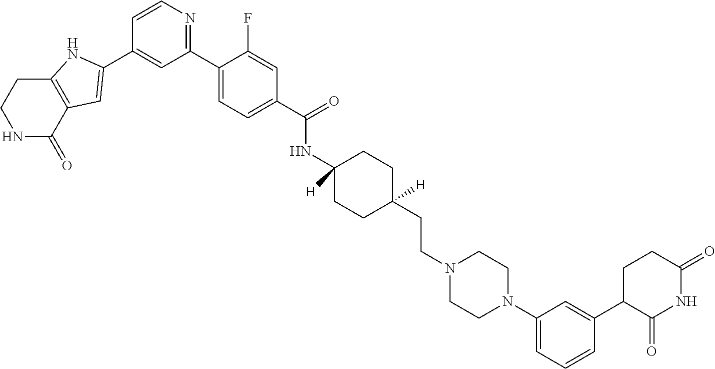 [SMILES: O=C1CCC(c2cccc(N3CCN(CC[C@H]4CC[C@H](NC(=O)c5ccc(-c6cc(-c7cc8c([nH]7)CCNC8=O)ccn6)c(F)c5)CC4)CC3)c2)C(=O)N1] | Ex 1.9 |
| 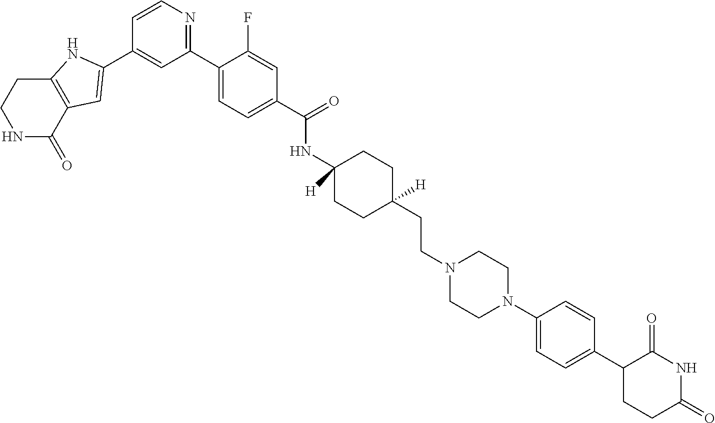 [SMILES: O=C1CCC(c2ccc(N3CCN(CC[C@H]4CC[C@H](NC(=O)c5ccc(-c6cc(-c7cc8c([nH]7)CCNC8=O)ccn6)c(F)c5)CC4)CC3)cc2)C(=O)N1] | Ex 1.10 |
| 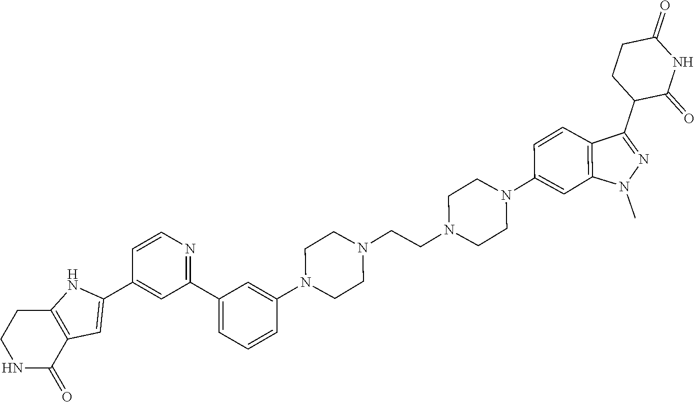 [SMILES: Cn1nc(C2CCC(=O)NC2=O)c2ccc(N3CCN(CCN4CCN(c5cccc(-c6cc(-c7cc8c([nH]7)CCNC8=O)ccn6)c5)CC4)CC3)cc21] | Ex 1.11 |
| 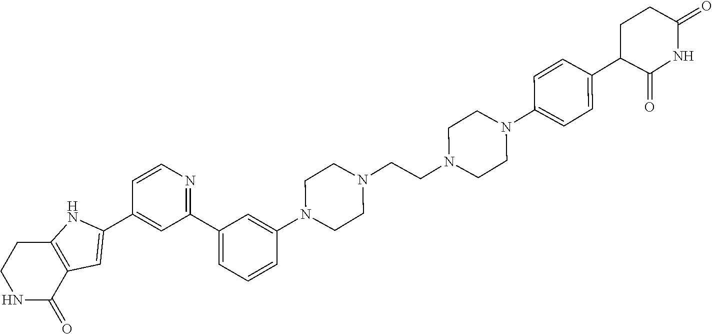 [SMILES: O=C1CCC(c2ccc(N3CCN(CCN4CCN(c5cccc(-c6cc(-c7cc8c([nH]7)CCNC8=O)ccn6)c5)CC4)CC3)cc2)C(=O)N1] | Ex 1.12 |
| 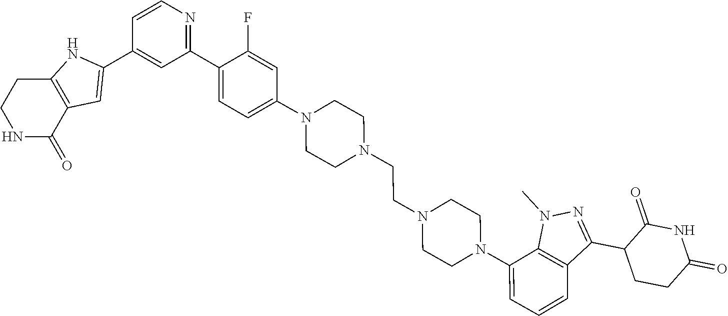 [SMILES: Cn1nc(C2CCC(=O)NC2=O)c2cccc(N3CCN(CCN4CCN(c5ccc(-c6cc(-c7cc8c([nH]7)CCNC8=O)ccn6)c(F)c5)CC4)CC3)c21] | Ex 1.13 |

or a pharmaceutically acceptable salt thereof.

Embodiment 30: A pharmaceutical composition comprising a compound according to any one of Embodiments 1-29 and a pharmaceutically acceptable excipient, carrier, or diluent.

Embodiment 31: A method of inhibiting the activity of MK2, or a mutant thereof, the method comprising contacting a biological sample with a compound according to any one of Embodiments 1-29.

Embodiment 32: A method of treating a disease, disorder, or condition mediated by MK2, or a mutant thereof, the method comprising administering to a patient in need thereof a compound according to any one of Embodiments 1-29, or a pharmaceutical composition according to Embodiment 30.

### EQUIVALENTS

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A compound of Formula I: 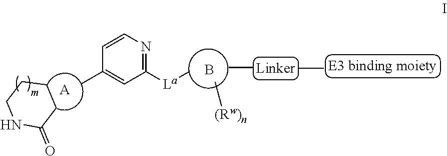
or a pharmaceutically acceptable salt thereof, wherein:
the Linker is a bivalent group selected from: an optionally substituted bivalent C₂₋₂₀ straight or branched aliphatic chain, wherein one, two, three, four, or five methylene units of the aliphatic chain are optionally and independently replaced by a group selected from —N(R)—, —C(O)—, —C(O)N(R)—, a 4- to 6-membered monocyclic saturated ring having 0-2 heteroatoms independently selected from nitrogen, a bivalent 6- to 8-membered saturated or partially unsaturated bridged bicyclic, or a fused bicyclic or spirofused heterocyclic ring having 1-3 heteroatoms independently selected from nitrogen, oxygen, and sulfur,
the Linker is optionally substituted with 0, 1, or 2 instances of R^{L}, and
R^{L} is independently selected from —OR, or an optionally substituted group selected from the group consisting of C16 aliphatic chain;
the E3 binding moiety is a moiety that binds to an E3 ubiquitin ligase protein;
Ring A is selected from 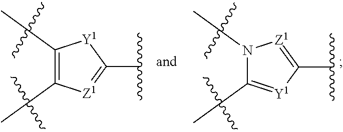
Y¹ is selected from —O—, —S—, and —N(R)—;
each of Y² and Z¹ is selected from —C(R¹)— and —N—;
R¹ is selected from hydrogen, halogen, and optionally substituted C₁₋₆ aliphatic;
Ring B is a phenylene, a 5- to 6-membered heteroarylene ring having 1-4 heteroatoms independently selected from nitrogen, oxygen, and sulfur, or a 3- to 7-membered saturated or partially unsaturated heterocyclylene having 1-3 heteroatoms independently selected from nitrogen, oxygen, and sulfur;
L^{a} is selected from a covalent bond, —O—, —S—, —N(R)—, and C₁₋₆ aliphatic;
R^{w} is selected from halogen, —OR, —SR, —CN, —NO₂, —SO₂NR, —SO₂R, —SOR, —C(O)R, —CO₂R, —C(O)N(R)₂, —NRC(O)R, —NRC(O)OR, —NRC(O)N(R)₂, —NRSO₂R, —N(R)₂, or an optionally substituted group selected from the group consisting of C₁₋₆ aliphatic, phenyl, a 3- to 8-membered saturated or partially unsaturated carbocyclic ring, a 4- to 7-membered heterocyclic ring having 1-2 heteroatoms independently selected from nitrogen, oxygen, or sulfur, and a 5- to 6-membered monocyclic heteroaryl ring having 1-4 heteroatoms independently selected from nitrogen, oxygen, or sulfur;
each R is independently at each occurrence hydrogen or optionally substituted C₁₋₆ aliphatic; and
each of m and n is 0, 1, 2, or 3.

2. The compound according to claim 1, wherein Ring A is

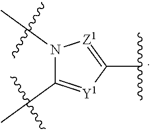

3. The compound according to claim 1, wherein the compound isa compound of Formula I-a 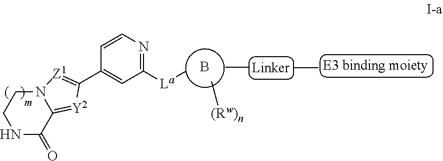
or a pharmaceutically acceptable salt thereof.

4. The compound according to claim 1, wherein Y² is —C(R¹)—.

5. The compound according to claim 1, wherein Z¹ is —C(R¹)—.

6. The compound according to claim 1, wherein Ring A is

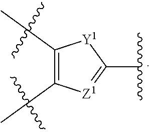

7. The compound according to claim 1, wherein the compound is a compound of Formula I-b: 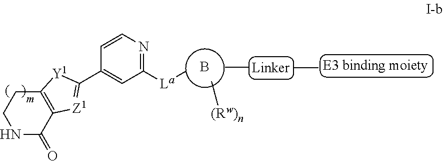
or a pharmaceutically acceptable salt thereof.

8. The compound according to claim 7, wherein Y¹ is —N(R)—.

9. The compound according to claim 7, wherein Z¹ is —C(R¹)—.

10. The compound according to claim 1, wherein Ring B is phenylene.

11. The compound according to claim 1, wherein L^{a} is a covalent bond.

12. The compound according to claim 1, wherein the compound is a compound of Formulae I-a-i, I-a-ii, I-a-iii, I-a-iv, I-b-i, I-b-ii, I-b-iii, and I-b-iv: 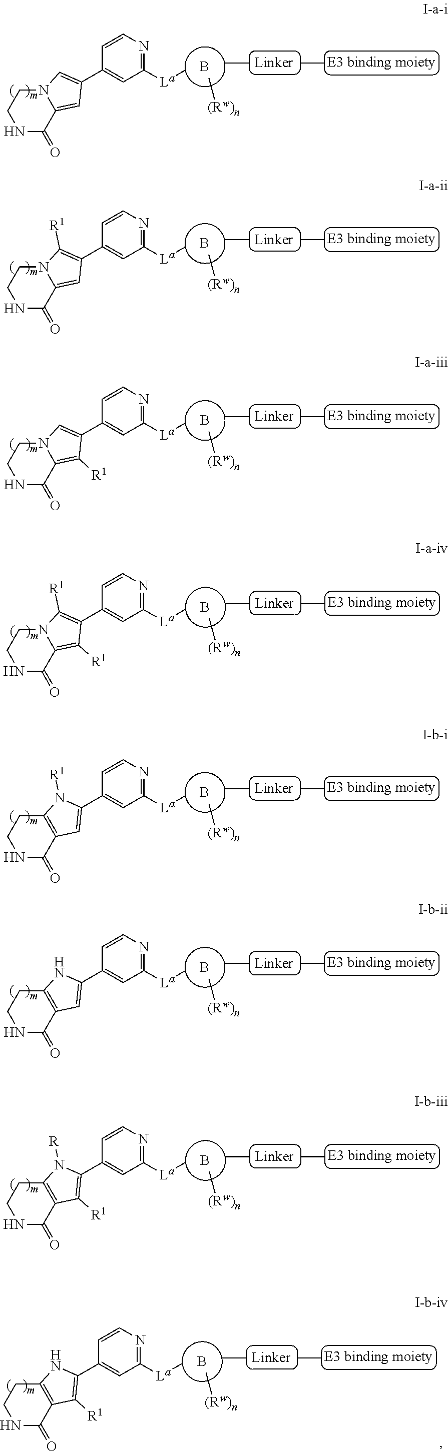
or a pharmaceutically acceptable salt thereof.

13. The compound according to claim 1, wherein:
the Linker is selected from the group consisting of:

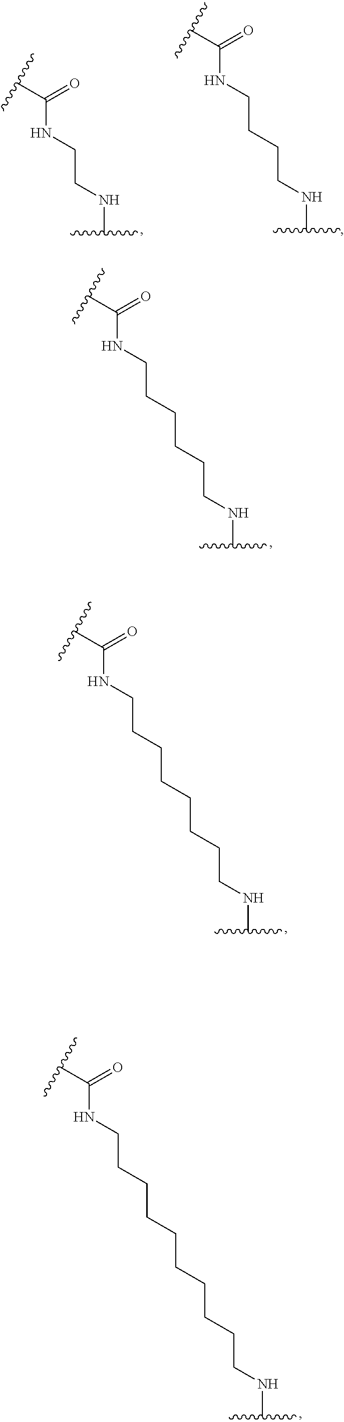 [SMILES: *NCCNC(*)=O ; *NCCCCNC(*)=O ; *NCCCCCCNC(*)=O ; *NCCCCCCCCNC(*)=O ; *NCCCCCCCCCCNC(*)=O]

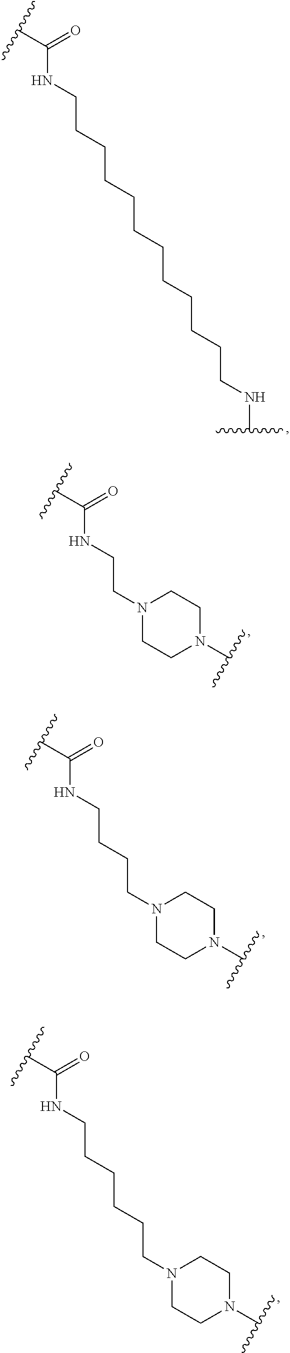 [SMILES: *NCCCCCCCCCCCCNC(*)=O ; *C(=O)NCCN1CCN(*)CC1 ; *C(=O)NCCCCN1CCN(*)CC1 ; *C(=O)NCCCCCCN1CCN(*)CC1]

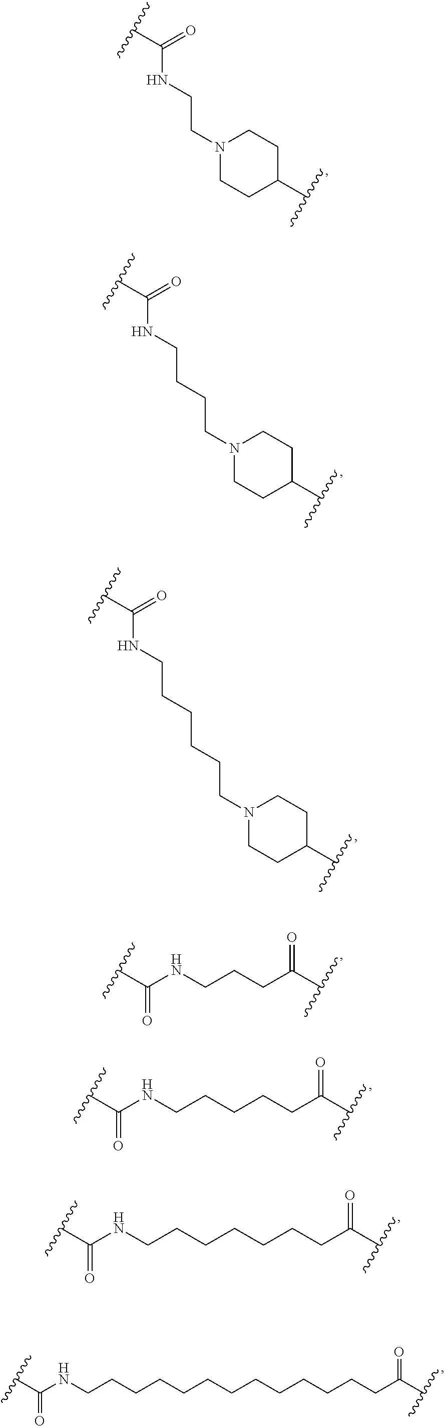

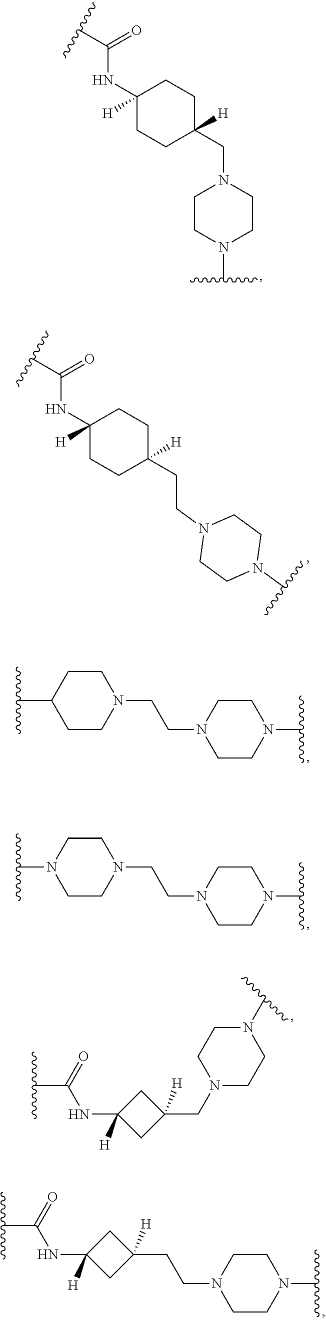 [SMILES: *C(=O)N[C@H]1CC[C@H](CN2CCN(*)CC2)CC1 ; *C(=O)N[C@H]1CC[C@H](CCN2CCN(*)CC2)CC1 ; *C1CCN(CCN2CCN(*)CC2)CC1 ; *N1CCN(CCN2CCN(*)CC2)CC1 ; *C(=O)N[C@H]1C[C@H](CN2CCN(*)CC2)C1 ; *C(=O)N[C@H]1C[C@H](CCN2CCN(*)CC2)C1]

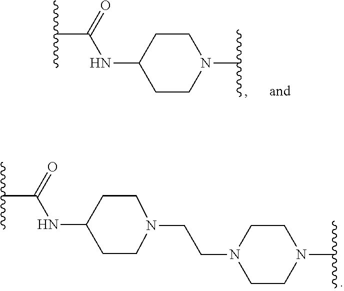 [SMILES: *C(=O)NC1CCN(*)CC1 ; *C(=O)NC1CCN(CCN2CCN(*)CC2)CC1]

14. The compound according to claim 1, wherein the Linker is selected from the group consisting of:

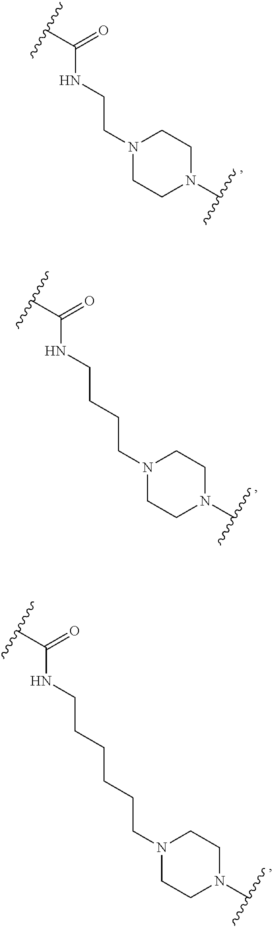 [SMILES: *C(=O)NCCN1CCN(*)CC1 ; *C(=O)NCCCCN1CCN(*)CC1 ; *C(=O)NCCCCCCN1CCN(*)CC1]

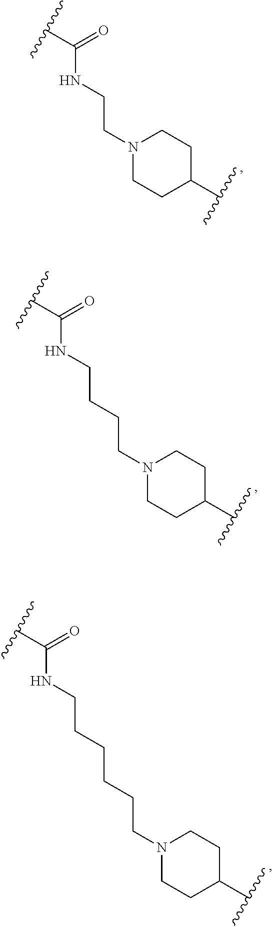 [SMILES: *C(=O)NCCN1CCC(*)CC1 ; *C(=O)NCCCCN1CCC(*)CC1 ; *C(=O)NCCCCCCN1CCC(*)CC1]

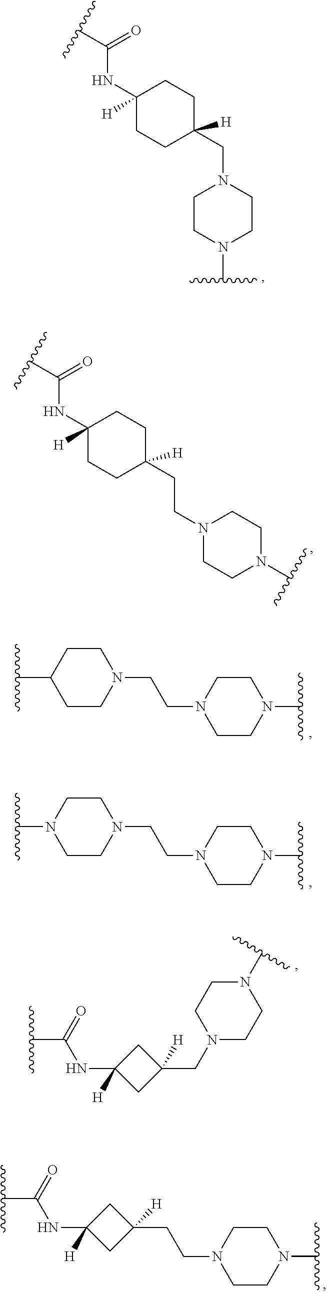 [SMILES: *C(=O)N[C@H]1CC[C@H](CN2CCN(*)CC2)CC1 ; *C(=O)N[C@H]1CC[C@H](CCN2CCN(*)CC2)CC1 ; *C1CCN(CCN2CCN(*)CC2)CC1 ; *N1CCN(CCN2CCN(*)CC2)CC1 ; *C(=O)N[C@H]1C[C@H](CN2CCN(*)CC2)C1 ; *C(=O)N[C@H]1C[C@H](CCN2CCN(*)CC2)C1]

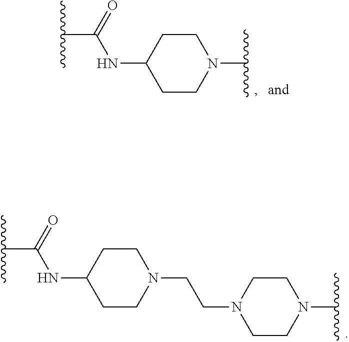 [SMILES: *C(=O)NC1CCN(*)CC1 ; *C(=O)NC1CCN(CCN2CCN(*)CC2)CC1]

15. The compound according to claim 1, wherein the E3 binding moiety is a cereblon protein binding moiety.

16. The compound according to claim 15, wherein the cereblon protein binding moiety is selected from the group consisting of:

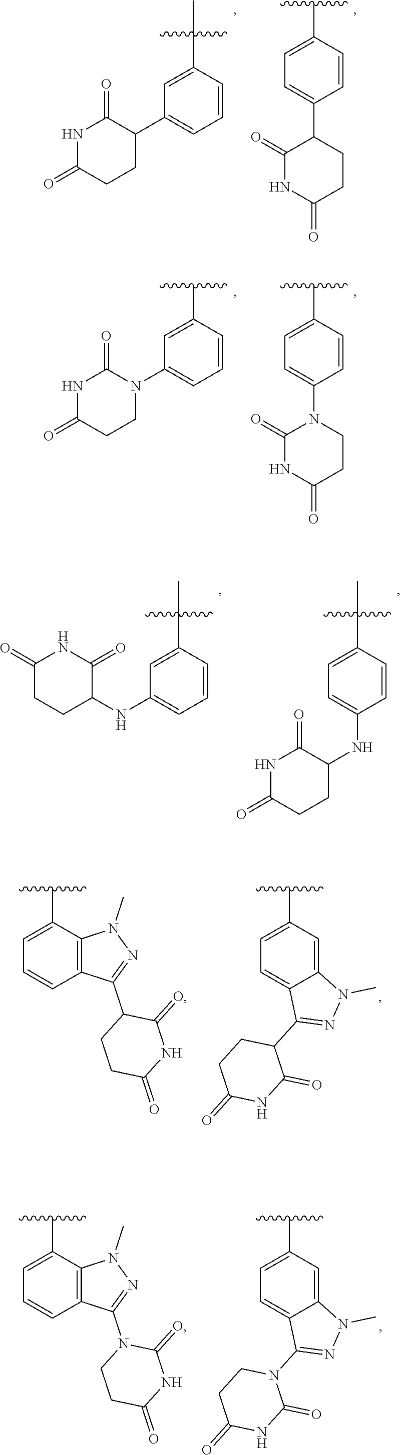

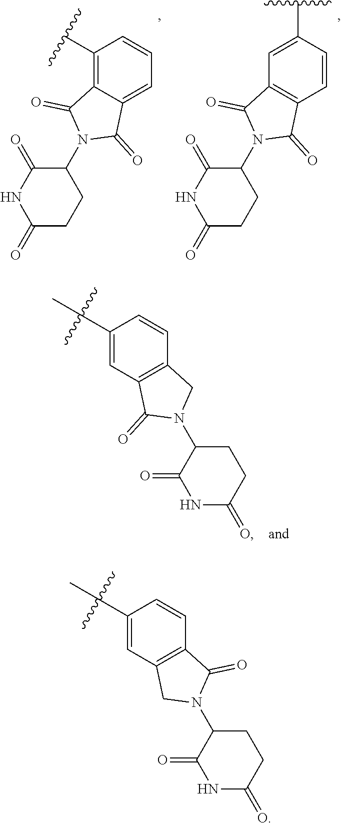 [SMILES: *c1cccc2c1C(=O)N(C1CCC(=O)NC1=O)C2=O ; *c1ccc2c(c1)C(=O)N(C1CCC(=O)NC1=O)C2=O ; *c1ccc2c(c1)C(=O)N(C1CCC(=O)NC1=O)C2 ; "and" ; *c1ccc2c(c1)CN(C1CCC(=O)NC1=O)C2=O]

17. The compound according to claim 1, wherein the compound is selected from Table 1:

| | |
|---|---|
| 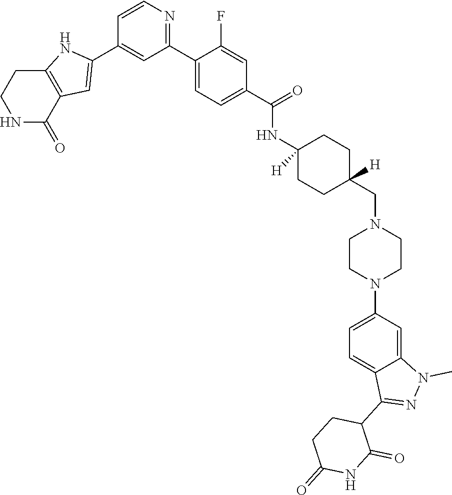 [SMILES: Cn1nc(C2CCC(=O)NC2=O)c2ccc(N3CCN(C[C@H]4CC[C@H](NC(=O)c5ccc(-c6cc(-c7cc8c([nH]7)CCNC8=O)ccn6)c(F)c5)CC4)CC3)cc21] | Ex. 1.5 |
| 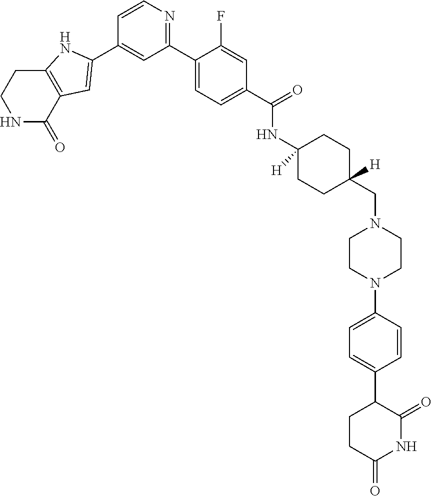 [SMILES: O=C1CCC(c2ccc(N3CCN(C[C@H]4CC[C@H](NC(=O)c5ccc(-c6cc(-c7cc8c([nH]7)CCNC8=O)ccn6)c(F)c5)CC4)CC3)cc2)C(=O)N1] | Ex 1.6 |
| 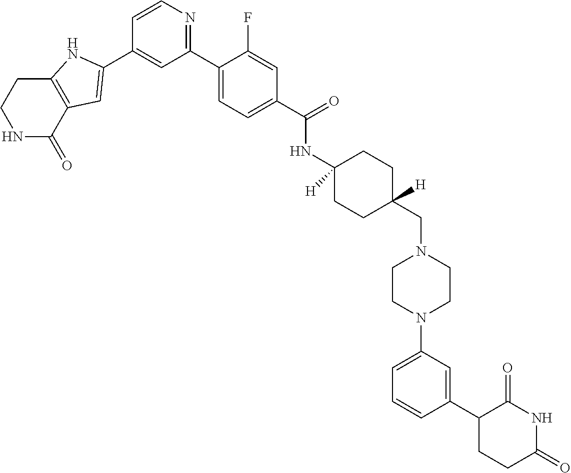 [SMILES: O=C1CCC(c2cccc(N3CCN(C[C@H]4CC[C@H](NC(=O)c5ccc(-c6cc(-c7cc8c([nH]7)CCNC8=O)ccn6)c(F)c5)CC4)CC3)c2)C(=O)N1] | Ex 1.7 |
| 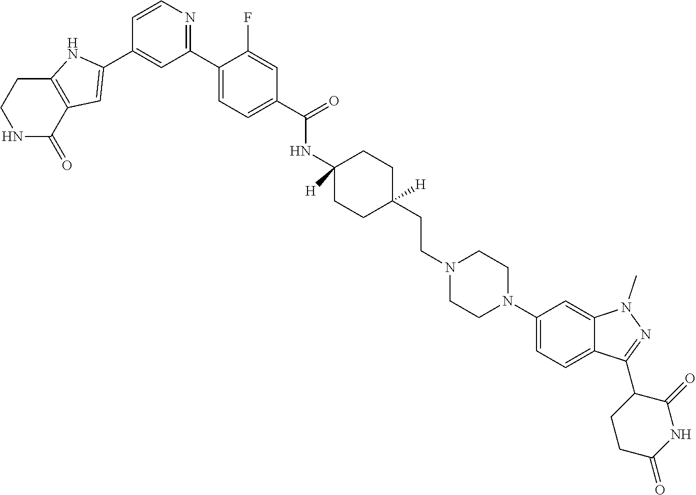 [SMILES: Cn1nc(C2CCC(=O)NC2=O)c2ccc(N3CCN(CC[C@H]4CC[C@H](NC(=O)c5ccc(-c6cc(-c7cc8c([nH]7)CCNC8=O)ccn6)c(F)c5)CC4)CC3)cc21] | Ex 1.8 |
| 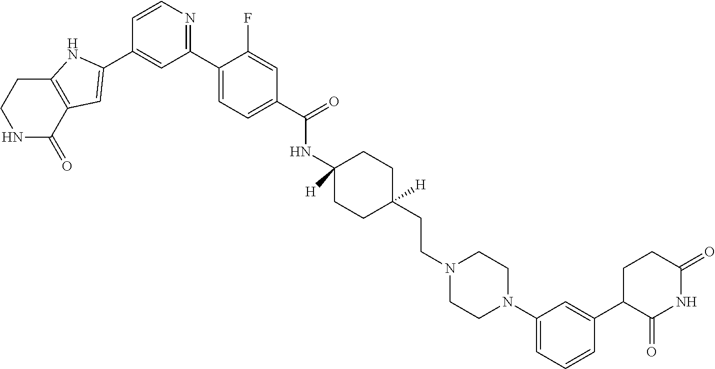 [SMILES: O=C1CCC(c2cccc(N3CCN(CC[C@H]4CC[C@H](NC(=O)c5ccc(-c6cc(-c7cc8c([nH]7)CCNC8=O)ccn6)c(F)c5)CC4)CC3)c2)C(=O)N1] | Ex 1.9 |
| 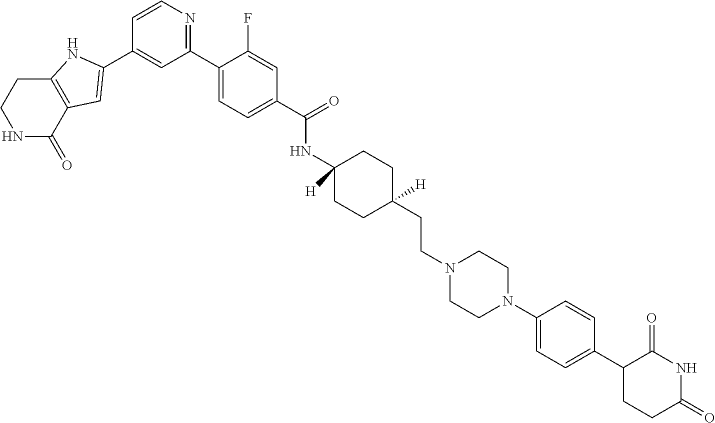 [SMILES: O=C1CCC(c2ccc(N3CCN(CC[C@H]4CC[C@H](NC(=O)c5ccc(-c6cc(-c7cc8c([nH]7)CCNC8=O)ccn6)c(F)c5)CC4)CC3)cc2)C(=O)N1] | Ex 1.10 |
| 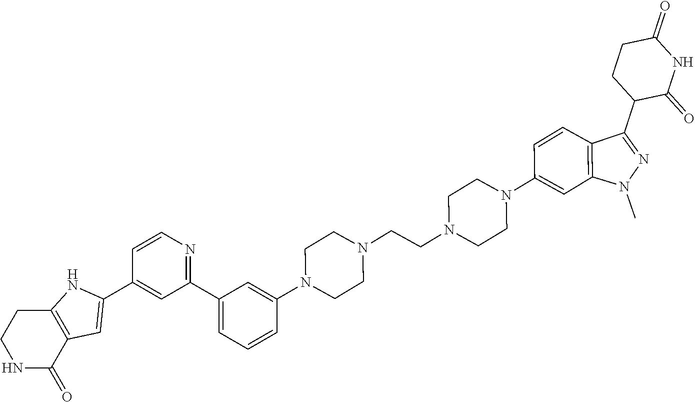 [SMILES: Cn1nc(C2CCC(=O)NC2=O)c2ccc(N3CCN(CCN4CCN(c5cccc(-c6cc(-c7cc8c([nH]7)CCNC8=O)ccn6)c5)CC4)CC3)cc21] | Ex 1.11 |
| 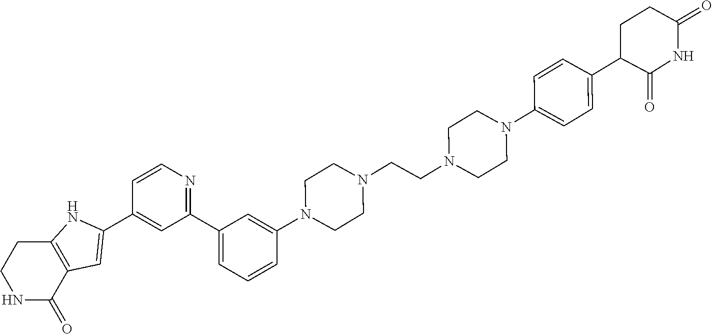 [SMILES: O=C1CCC(c2ccc(N3CCN(CCN4CCN(c5cccc(-c6cc(-c7cc8c([nH]7)CCNC8=O)ccn6)c5)CC4)CC3)cc2)C(=O)N1] | Ex 1.12 |
| 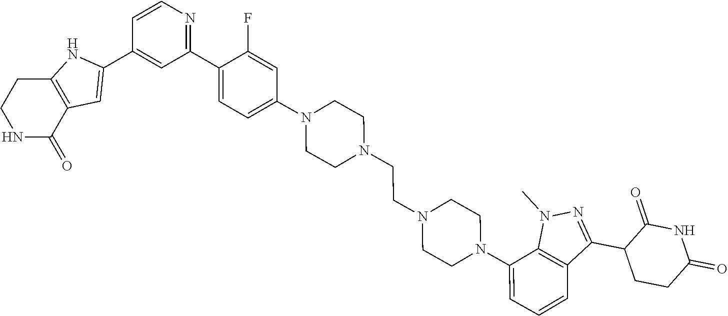 [SMILES: Cn1nc(C2CCC(=O)NC2=O)c2cccc(N3CCN(CCN4CCN(c5ccc(-c6cc(-c7cc8c([nH]7)CCNC8=O)ccn6)c(F)c5)CC4)CC3)c21] | Ex 1.13 |

or a pharmaceutically acceptable salt thereof.

18. A pharmaceutical composition comprising a compound according to claim 1, and a pharmaceutically acceptable excipient, carrier, or diluent.

19. A method of inhibiting the activity of MK2, or a mutant thereof the method comprising contacting a biological sample with a compound according to claim 1.

20. A method of treating a disease, disorder, or condition mediated by MK2, or a mutant thereof, the method comprising administering to a patient in need thereof a compound according to claim 1.


SEQUENCE LISTING (1 sequence)

SEQ ID NO: 1 - protein, 400 aa, Homo sapiens
MLSNSQGQSPPVPFPAPAPPPQPPTPALPHPPAQPPPPPPQQFPQFHVKSGLQIKKNAIIDDYKVTSQVLGLGINGKVLQIFNKRTQEKFALKMLQDCPKARREVELHWRASQCPHIVRIVDVYENLYAGRKCLLIVMECLDGGELFSRIQDRGDQAFTEREASEIMKSIGEAIQYLHSINIAHRDVKPENLLYTSKRPNAILKLTDFGFAKETTSHNSLTTPCYTPYYVAPEVLGPEKYDKSCDMWSLGVIMYILLCGYPPFYSNHGLAISPGMKTRIRMGQYEFPNPEWSEVSEEVKMLIRNLLKTEPTQRMTITEFMNHPWIMQSTKVPQTPLHTSRVLKEDKERWEDVKEEMTSALATMRVDYEQIKIKKIEDASNPLLLKRRKKARALEAAALAH